(19) United States
(12) Patent Application Publication
(10) Pub. No.: US 2026/0258055 A1
(43) Pub. Date: Sep. 3, 2026

(54) CRYSTALLINE FORMS AND FORMULATIONS OF A KRAS INHIBITOR

(71) Applicant: Eli Lilly and Company, Indianapolis, IN (US)

(72) Inventors: Robert William BONDI, Jr., Lynnfield, MA (US); David Andrew COATES, New Palestine, IN (US); Andrew Ralph COOKE, Lafayette, CO (US); Samir KULKARNI, Carmel, IN (US); Montgomery LAKY, Opelika, AL (US); Christopher M. LINDEMANN, Fort Collins, CO (US); David Charles LITZINGER, Poway, CA (US); Prashant Kumar PANDEY, Indianapolis, IN (US); Samir Chandubhai PATEL, Erie, CO (US); Sofiane SAOUANE, Indianapolis, IN (US); Ruchit TRIVEDI, Redondo Beach, CA (US)

(21) Appl. No.: 19/552,123

(22) Filed: Feb. 27, 2026

Related U.S. Application Data
(60) Provisional application No. 63/765,158, filed on Feb. 28, 2025.

(51) Int. Cl. C07D 519/00; A61K 9/00; A61K 9/48; A61K 31/517; A61K 31/519; A61K 45/06; C07D 275/06

(52) U.S. Cl. CPC C07D 519/00; A61K 9/0053; A61K 9/4858; A61K 9/4866; A61K 9/4891; A61K 31/517; A61K 31/519; A61K 45/06; C07D 275/06

(57) ABSTRACT

The present disclosure relates to crystalline forms of 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile and pharmaceutical formulations comprising the compound or a pharmaceutically acceptable salt thereof, methods of making, and use for the treatment of disease.

### TECHNICAL FIELD

The present disclosure is directed to crystalline forms of 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile and pharmaceutical compositions/formulations comprising the compound or a pharmaceutically acceptable salt thereof, and use thereof for the treatment of diseases.

### BACKGROUND

Therapeutic agents can be administered to patients via different routes, including oral, intravenous, and subcutaneous. Oral dosing of therapeutics offers multiple advantages over other routes of administration. For example, orally delivered drugs are easily self-administered, thereby resulting in increased patient compliance and obviating the requirement for specialized delivery devices for injectable or inhaled therapies or delivery in a therapeutic setting. Moreover, oral administration is typically the safest route of getting a drug into the body since it does not require complicated devices or puncturing of body surfaces or membranes.

Despite the advantages offered by oral dosing, obtaining consistent and adequate circulating levels of drug with oral dosing can be challenging due to, among other things: poor aqueous solubility; slow dissolution rate in biological fluids; poor stability of drug at physiological pH; poor permeation through biomembranes; extensive presystemic metabolism; and inadequate or inconsistent systemic absorption between individuals or within specific regions of the gastrointestinal system. From a safety standpoint, minimizing the total dosage requirement for efficacy as well as reducing variability in absorption should allow for fewer unwanted side effects such as diarrhea and emesis, commonly called vomiting.

Oncogenic KRAS mutations have been identified in approximately 30% of human cancers and have been demonstrated to activate multiple downstream signaling pathways. Despite the prevalence of KRAS mutations, it has been a difficult therapeutic target. (Cox, A. D. Drugging the Undruggable RAS: Mission Possible? Nat. Rev. Drug Disc. 2014, 13, 828-851; Pylayeva-Gupta, y et al. RAS Oncogenes: Weaving a Tumorigenic Web. Nat. Rev. Cancer 2011, 11, 761-774).

Thus far, work has focused on KRAS G12C mutant inhibitors (e.g., WO2020/081282, WO2020/101736, WO2020/146613, and WO2021/118877 disclose KRAS G12C inhibitors), whereas WO2021/041671 discloses small molecules inhibitors of KRAS G12D and WO2017/011920 discloses small molecule inhibitors of KRAS G12C, G12D, and G12V.

WO2023/183585 discloses KRAS G12D inhibitors. An example of a KRAS G12D inhibitor is 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile, which has the following structure:

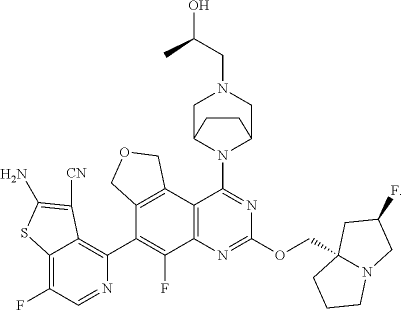 [SMILES: C[C@@H](O)CN1CC2CCC(C1)N2c1nc(OC[C@@]23CCCN2C[C@H](F)C3)nc2c(F)c(-c3ncc(F)c4sc(N)c(C#N)c34)c3c(c12)COC3]

This compound is disclosed in WO2023/183585 as an amorphous, free base compound (hereinafter “Formula II”).

There remains a need to provide a more soluble form of the compound of Formula II. In particular, there is a need to provide alternate methods of preparing the compound. Further, there is a need to provide pharmaceutical compositions comprising the compound or a pharmaceutically acceptable salt thereof that provide an improved pharmacokinetic profile with reduced or minimized untoward or undesired effects, such as emesis or diarrhea. The present invention addresses one or more of these needs by providing novel crystalline forms of the compound and/or pharmaceutical compositions comprising the compound or a pharmaceutically acceptable salt thereof.

### SUMMARY

In one embodiment, provided is a saccharinate salt compound of

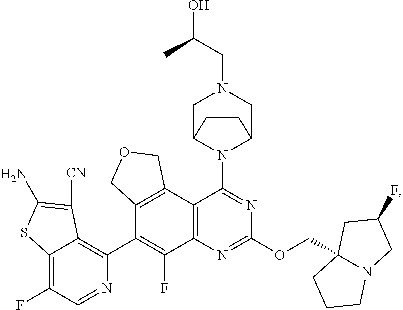 [SMILES: C[C@@H](O)CN1CC2CCC(C1)N2c1nc(OC[C@@]23CCCN2C[C@H](F)C3)nc2c(F)c(-c3ncc(F)c4sc(N)c(C#N)c34)c3c(c12)COC3]

wherein solvent may be optionally present. In some embodiments, the solvent is selected from water, tetrahydrofuran (THF), acetone, methanol, ethanol, and benzyl alcohol.

In another embodiment, the present disclosure is directed to a compound of Formula I:

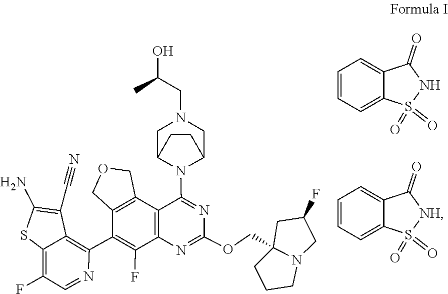

wherein solvent may be optionally present.
   The compound of Formula I is a disaccharinate salt and the chemical name for Formula I can be written as 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate. In some embodiments, the compound of Formula I comprises one or more solvents, such as water, tetrahydrofuran (THF), acetone, methanol, ethanol, and benzyl alcohol.

In another embodiment, the present disclosure is directed to a method of preparing a compound of Formula I, the method comprising suspending 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (i.e., Formula II) in a solvent, adding saccharin and isolating the compound of Formula I.

In another embodiment, the present disclosure is directed to a solid oral pharmaceutical dosage form comprising: a) a core composition comprising a compound disclosed herein (e.g., Formula I, Formula II); b) a capsule that contains the core composition and wherein the capsule has a body and a cap: c) a polymeric seal covering the transition between the capsule cap and body; and d) an enteric coating that coats the polymeric seal and capsule.

In another embodiment, the present disclosure is directed to a process for preparing the dosage form of a compound disclosed herein (e.g., Formula I, Formula II), comprising: a) blending all components comprised in the core composition in a suitable blender; b) weighing an amount of the blend formed in step (a) to be the core composition; c) compressing the blend weighed in step (b) using a capsule slug mold; d) placing the slug formed in step (c) into the capsule; e) covering the transition between the capsule cap and body with the polymeric seal; and f) coating the capsule with the enteric coating.

In another embodiment, the present disclosure is directed to a solid pharmaceutical composition, comprising: (1) a compound disclosed herein constituting about 30-67% percent of the composition; (2) microcrystalline cellulose constituting about 20-55% of the composition; (3) mannitol constituting about 0-10% of the composition; (4) crospovidone constituting about 2% to about 5% of the composition; (5) colloidal silicon dioxide constituting up to about 0.5-2% of the composition; (6) magnesium stearate constituting about 1-3% of the composition, and (7) an enteric coating, wherein all percentages are percentages by weight and wherein the total weight is 100%. In some embodiments, the solid pharmaceutical composition is in the form of a tablet.

In another embodiment, provided are therapies including the compounds disclosed herein, or pharmaceutically acceptable salts thereof, for the treatment of diseases, such as cancer.

In another embodiment, provided are therapies including the pharmaceutical compositions disclosed herein, for the treatment of diseases, such as cancer.

### BRIEF DESCRIPTION OF THE FIGURES

FIG. 1 is an XRPD chromatogram of 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile 1.25 acetate 0.75 hydrate.

FIG. 2 is an XRPD chromatogram of 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile monocinnamate.

FIG. 3 is an XRPD chromatogram of Formula I.

FIG. 4 shows data from an in vivo pharmacokinetic study of enteric and non-enteric capsules comprising Formula I administered orally (60 mg/kg) to monkeys. The free plasma concentration of Formula I versus time (averaged for three animals) is shown.

FIG. 5 shows data from an in vivo pharmacokinetic study of non-enteric capsules comprising Formula I administered orally (60 mg/kg) to monkeys. The free plasma concentration of Formula I versus time (for individual animals) is shown.

FIG. 6 shows data from an in vivo pharmacokinetic study of enteric capsules comprising Formula I administered orally (60 mg/kg) to monkeys. The free plasma concentration of Formula I versus time (for individual animals) is shown.

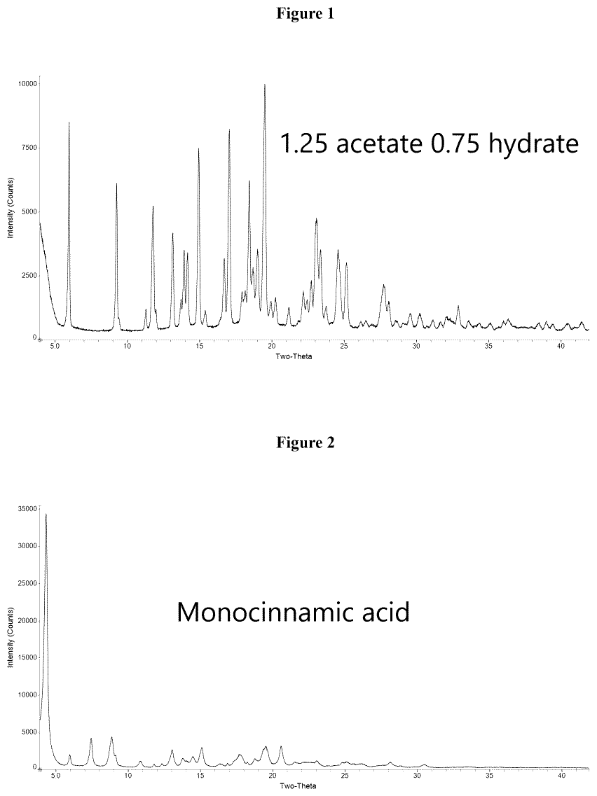

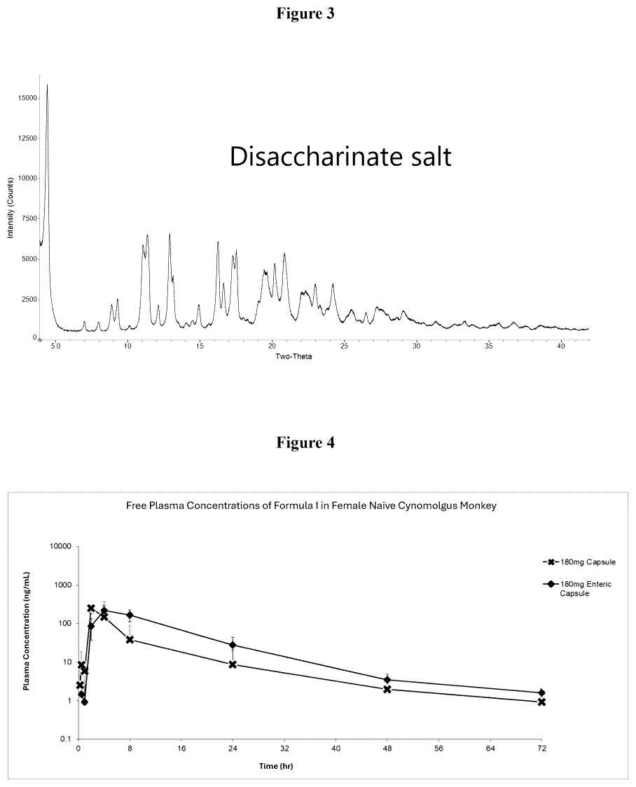

### DETAILED DESCRIPTION

Disclosed herein are crystalline forms of

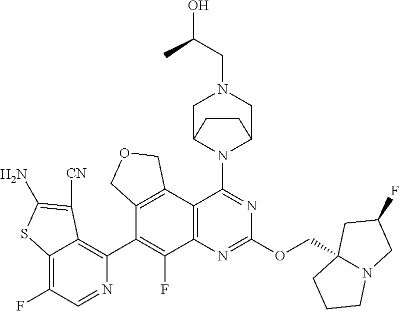 [SMILES: C[C@@H](O)CN1CC2CCC(C1)N2c1nc(OC[C@@]23CCCN2C[C@H](F)C3)nc2c(F)c(-c3ncc(F)c4sc(N)c(C#N)c34)c3c(c12)COC3]

and alternate methods of preparing the crystalline forms of the compound. Further, the present disclosure is directed to pharmaceutical compositions comprising the compounds disclosed herein or pharmaceutically acceptable salts thereof (e.g., Formula I, Formula II) that provide an improved pharmacokinetic profile with reduced or minimized untoward or undesired effects, such as emesis or diarrhea.

In some embodiments, the present disclosure provides pharmaceutical compositions (e.g., enteric capsule, enteric tablet) comprising a compound disclosed herein (e.g., Formula I, Formula II). In some embodiments, the pharmaceutical compositions disclosed herein may reduce the frequency of emesis without substantial impact on exposure by delivering the compound disclosed herein (e.g., Formula I, Formula II) to the upper-gastrointestinal tract. In some embodiments, the pharmaceutical compositions comprising a compound disclosed herein show improved pharmacokinetics (e.g., lower compound variability and higher exposure of the compound in the subjects). In some embodiments, no emesis (or reduced frequency of emesis) is observed in the subjects that receive the pharmaceutical compositions comprising a compound disclosed herein (e.g., Formula I, Formula II).

As used herein, “compound disclosed herein” or “compounds disclosed herein” refer to any compound disclosed in the present disclosure, including Formula I, Formula II (free base), and pharmaceutically acceptable salts of Formula II. Exemplary salts of the compounds disclosed herein include saccharinate salts, such as the disaccharinate salt. In some embodiments, the compound disclosed herein optionally comprises one or more solvents. In some embodiments, the one or more solvents are selected from water, tetrahydrofuran (THF), acetone, methanol, ethanol, and benzyl alcohol.

In some embodiments, the compounds disclosed herein (e.g., Formula I) can be used to treat cancer, including KRAS G12D associated cancers.

A KRAS G12D associated cancer is a cancer wherein one or more cancer cells express a KRAS G12D mutant protein or contain a KRAS G12D mutation. Examples of cancers that can be treated using the compounds disclosed herein include, but are not limited to lung cancer, pancreatic cancer, cervical cancer, esophageal cancer, endometrial cancer, ovarian cancer, cholangiocarcinoma, and colorectal cancer. More specific, but not limiting examples of KRAS G12D associated cancers that can be treated using the compounds disclosed herein (e.g., Formula I) include pancreatic adenocarcinoma, colon adenocarcinoma, lung adenocarcinoma, colorectal adenocarcinoma, and rectal adenocarcinoma. In one embodiment, the cancer is non-small cell lung cancer. In another embodiment, the cancer is colorectal cancer. In still another embodiment, the cancer is pancreatic cancer. In another embodiment, the cancer is gastric cancer.

In one embodiment, the KRAS G12D associated cancer is a solid tumor that contains one or more cancer cells that express a KRAS G12D mutant protein or contain a KRAS G12D mutation.

In one embodiment, disclosed is a method of treating a solid tumor that contains one or more cancer cells that express a KRAS G12D mutant protein or contain a KRAS G12D mutation, wherein the method comprises administering a compound disclosed herein (e.g., Formula I) to a patient in need of such treatment.

In one embodiment, the patient is also administered an effective amount of one or more of a PD-1 inhibitor, a PD-L1 inhibitor, a CDK4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof. An example of a PD-1 inhibitor is pembrolizumab. An example of a CDK4/CDK6 inhibitor is abemaciclib. An example of an EGFR inhibitor is cetuximab. In one embodiment, the compound disclosed herein (e.g., Formula I) is administered in simultaneous, separate, or sequential combination with an effective amount of PD-1 inhibitor. In one embodiment, the compound (e.g., Formula I) is administered in simultaneous, separate, or sequential combination with an effective amount of PD-1 inhibitor that comprises pembrolizumab. In one embodiment, the compound (e.g., Formula I) is administered in simultaneous, separate, or sequential combination with an effective amount of a PD-L1 inhibitor. In one embodiment, the compound (e.g., Formula I) is administered in simultaneous, separate, or sequential combination with an effective amount of a CDK4/CDK6 inhibitor. In one embodiment, the compound (e.g., Formula I) is administered in simultaneous, separate, or sequential combination with an effective amount of a CDK4/CDK6 inhibitor that comprises abemaciclib. In one embodiment, the compound (e.g., Formula I) is administered in simultaneous, separate, or sequential combination with an effective amount of an EGFR inhibitor. In one embodiment, the compound (e.g., Formula I) is administered in simultaneous, separate, or sequential combination with an effective amount of an EGFR inhibitor that comprises cetuximab. In one embodiment, the compound (e.g., Formula I) is administered in simultaneous, separate, or sequential combination with an effective amount of an ERK inhibitor. In one embodiment, the compound (e.g., Formula I) is administered in simultaneous, separate, or sequential combination with an effective amount of an Aurora A inhibitor. In one embodiment, the compound (e.g., Formula I) is administered in simultaneous, separate, or sequential combination with an effective amount of a SHP2 inhibitor. In one embodiment, the compound (e.g., Formula I) is administered in simultaneous, separate, or sequential combination with an effective amount of a platinum agent. In one embodiment, the compound (e.g., Formula I) is administered in simultaneous, separate, or sequential combination with an effective amount of pemetrexed.

Disclosed herein is a method of treating gastric cancer, the method comprising administering a compound disclosed herein (e.g., Formula I) to a patient in need of such treatment.

Disclosed herein is a method of treating a G12D associated cancer, the method comprising administering a compound disclosed herein (e.g., Formula I) to a patient in need of such treatment.

Disclosed herein is a method of treating a G12D associated cancer, wherein the cancer comprises lung cancer, pancreatic cancer, cervical cancer, esophageal cancer, endometrial cancer, ovarian cancer, cholangiocarcinoma, or colorectal cancer, the method comprising administering a compound disclosed herein (e.g., Formula I) to a patient in need of such treatment.

Disclosed herein is a method of treating gastric cancer, the method comprising administering a compound disclosed herein (e.g., Formula I) and a PD-1 inhibitor to a patient in need of such treatment. In an embodiment, the PD-1 inhibitor comprises pembrolizumab.

Disclosed herein is a method of treating a G12D associated cancer, the method comprising administering a compound disclosed herein (e.g., Formula I) and a PD-1 inhibitor to a patient in need of such treatment. In an embodiment, the PD-1 inhibitor comprises pembrolizumab.

Disclosed herein is a method of treating a G12D associated cancer, wherein the cancer comprises lung cancer, pancreatic cancer, cervical cancer, esophageal cancer, endometrial cancer, ovarian cancer, cholangiocarcinoma, or colorectal cancer, the method comprising administering a compound disclosed herein (e.g., Formula I) and a PD-1 inhibitor to a patient in need of such treatment. In an embodiment, the PD-1 inhibitor comprises pembrolizumab.

Disclosed herein is a method of treating gastric cancer, the method comprising administering a compound disclosed herein (e.g., Formula I) and an EGFR inhibitor to a patient in need of such treatment. In an embodiment, the EGFR inhibitor comprises cetuximab.

Disclosed herein is a method of treating a G12D associated cancer, the method comprising administering a compound disclosed herein (e.g., Formula I) and an EGFR inhibitor to a patient in need of such treatment. In an embodiment, the EGFR inhibitor comprises cetuximab.

Disclosed herein is a method of treating a G12D associated cancer, wherein the cancer comprises lung cancer, pancreatic cancer, cervical cancer, esophageal cancer, endometrial cancer, ovarian cancer, cholangiocarcinoma, or colorectal cancer, the method comprising administering a compound disclosed herein (e.g., Formula I) and an EGFR inhibitor to a patient in need of such treatment. In an embodiment, the EGFR inhibitor comprises cetuximab.

Disclosed herein is a method of treating gastric cancer, the method comprising administering a compound disclosed herein (e.g., Formula I) with a CDK4/CDK6 inhibitor to a patient in need of such treatment. In an embodiment, the CDK4/CDK6 inhibitor comprises abemaciclib.

Disclosed herein is a method of treating a G12D associated cancer, the method comprising administering a compound disclosed herein (e.g., Formula I) with a CDK4/CDK6 inhibitor to a patient in need of such treatment. In an embodiment, the CDK4/CDK6 inhibitor comprises abemaciclib.

Disclosed herein is a method of treating a G12D associated cancer, wherein the cancer comprises lung cancer, pancreatic cancer, cervical cancer, esophageal cancer, endometrial cancer, ovarian cancer, cholangiocarcinoma, or colorectal cancer, the method comprising administering a compound disclosed herein (e.g., Formula I) with a CDK4/CDK6 inhibitor to a patient in need of such treatment. In an embodiment, the CDK4/CDK6 inhibitor comprises abemaciclib.

Disclosed herein is a method of treating gastric cancer, the method comprising administering a compound disclosed herein (e.g., Formula I) with a PD-1 inhibitor and an EGFR inhibitor to a patient in need of such treatment. In an embodiment, the PD-1 inhibitor comprises pembrolizumab and the EGFR inhibitor comprises cetuximab.

Disclosed herein is a method of treating a G12D associated cancer, the method comprising administering a compound disclosed herein (e.g., Formula I) with a PD-1 inhibitor and an EGFR inhibitor to a patient in need of such treatment. In an embodiment, the PD-1 inhibitor comprises pembrolizumab and the EGFR inhibitor comprises cetuximab.

Disclosed herein is a method of treating a G12D associated cancer, wherein the cancer comprises lung cancer, pancreatic cancer, cervical cancer, esophageal cancer, endometrial cancer, ovarian cancer, cholangiocarcinoma, or colorectal cancer, the method comprising administering a compound disclosed herein (e.g., Formula I) with a PD-1 inhibitor and an EGFR inhibitor to a patient in need of such treatment. In an embodiment, the PD-1 inhibitor comprises pembrolizumab and the EGFR inhibitor comprises cetuximab.

Disclosed herein is a method of treating gastric cancer comprising administering a compound disclosed herein (e.g., Formula I) to a patient in need of such treatment. In an embodiment, the gastric cancer is KRAS G12D gastric cancer.

Disclosed herein is a method of treating a KRAS G12D associated cancer, the method comprising administering a compound disclosed herein (e.g., Formula I) to a patient in need of such treatment, wherein the KRAS G12D associated cancer comprises colorectal cancer, pancreatic cancer and non-small cell lung cancer.

In all methods and uses described herein, wherein two or more therapeutic agents are administered, the two or more therapeutic agents may be administered simultaneously, separately, or sequentially.

Pharmaceutical compositions containing the compounds disclosed herein (e.g., Formula I) may be prepared by combining the disclosed compounds with a pharmaceutically acceptable excipient. The term “excipient” refers to diluents or fillers, binders, granulating agents, adhesives, polymers and copolymers, disintegrants, stabilizers, lubricants, anti-adherents, glidants, surfactants, dispersing or wetting agents, dissolution retardants or enhancers, adsorbents, buffers, chelating agents, preservatives, colors, flavors, and sweeteners, or combinations thereof that are not deleterious to the patient. Examples of pharmaceutical compositions and processes for their preparation can be found in “Remington: The Science and Practice of Pharmacy”, Loyd, V., et al. Eds., 22^{nd} Ed., Mack Publishing Co., 2012.

In some embodiments of the solid formulation, the excipient comprises a diluent or filler. In some embodiments, the diluent or filler is selected from the group consisting of dibasic calcium phosphate, kaolin, lactose, dextrose, magnesium carbonate, sucrose, mannitol, glucose or other monosaccharaides, dextrin or other polysaccharides, microcrystalline cellulose, powdered cellulose, cellulose derivatives—such as HPMC, precipitated calcium carbonate, calcium sulfate, sorbitol, inositol, and starch, or combinations of two or more thereof. In some embodiments, the diluent or filler is present in an amount of about 5 wt % to about 95 wt %, or about 10 to about 90 wt %, or about 20 wt % to about 85 wt %, or about 25 wt % to about 70 wt %.

Disclosed herein are orally administrable, pharmaceutical compositions comprising the compounds disclosed herein (e.g. Formula I). In one embodiment, the orally administrable, pharmaceutical compositions are enterically coated. The enterically coated compositions provide at least one of greater bioavailability or reduced stomach upset, when compared to orally administrable, non-enterically coated pharmaceutical compositions. The compositions disclosed herein may be administered to a patient in either a fasted or a fed state. Therefore, in some embodiments, a compound of any of the embodiments is administered to a subject prior to a meal. In some such embodiments, the compound is administered 3 hours, 2 hours, 1 hour, 30 minutes, or 15 minutes before a meal. In other embodiments, a compound of any embodiments set forth herein is administered to a subject during a meal. In other embodiments, a compound of any of the embodiments described herein is administered to a subject within 3 hours, within 2 hours, within 1 hour, within 30 minutes, or within 15 minutes of a meal.

Disclosed herein are 1) enterically coated capsules, 2) enterically coated tablets, and 3) enterically coated pellets, wherein each of these comprises the compounds disclosed herein. In one embodiment, the compound is Formula I.

In one embodiment, the pharmaceutical formulation is an enteric capsule that contains Formula I, without an excipient. In one embodiment, the pharmaceutical formulation is an enteric tablet that contains Formula I.

In some embodiments, the pharmaceutical composition comprising neat active pharmaceutical ingredient (API), such as Formula I, may be filled into capsules that are made from acid resistant materials such as Eudracaps® enteric capsules and Capsugel® Enprotect® capsules. In some embodiments, the pharmaceutical formulation (e.g., enteric capsule) may be administered in a fasted or non-fasted state.

In some embodiments, the enteric capsules comprising a compound disclosed herein (e.g., Formula I) may significantly reduce emesis in fasted and non-fasted subjects when compared to HPMC capsules comprising a compound disclosed herein. In some embodiments, the enteric capsules comprising a compound disclosed herein (e.g., Formula I) provide a higher area under the curve (AUC) exposure and much lower variability for the compound disclosed herein (e.g., Formula I) when compared to HPMC capsules comprising the same compound disclosed herein. In some embodiments, no emesis is observed in the subjects receiving the enteric capsules comprising a compound disclosed herein (e.g., Formula I). In some embodiments, the subjects dosed with enteric capsules comprising a compound disclosed herein (e.g., Formula I) show a similar PK profile (e.g., lower drug variability between subjects when same dose is administered) and reduced and/or no emesis regardless of whether the subject is dosed in non-fasted or fasted state.

In some embodiments, the dosing of the enteric capsules with food may help mitigate nausea/vomiting.

In some embodiments, the dosing of the enteric tablets with food may help mitigate nausea/vomiting.

In some embodiments, the present disclosure is directed to a method for treating cancer in a subject in need thereof, the method comprising administering to the subject a therapeutically effective amount of the solid pharmaceutical composition disclosed herein (e.g., enteric tablet, enteric capsule). In some embodiments, the cancer is a KRAS G12D-associated cancer. In some embodiments, a side effect of vomiting in the subject is less than a side effect of vomiting associated with administering a non-enteric capsule composition of the same amount of Formula I. In some embodiments, a side effect of diarrhea in the subject is less than a side effect of diarrhea associated with administering a capsule composition of the same amount of Formula I. In some embodiments, a side effect of vomiting or diarrhea in the subject occurs in less than 5% of the total subject population. In some embodiments, a side effect of vomiting or diarrhea in the subject occurs in less than 10% of the total subject population. In some embodiments, a side effect of vomiting or diarrhea in the subject occurs in less than 15% of the total subject population. In some embodiments, a side effect of vomiting or diarrhea in the subject occurs in less than 20% of the total subject population. In some embodiments, a side effect of vomiting or diarrhea in the subject occurs in less than 25% of the total subject population.

### Enteric Coated Pharmaceutical Compositions

Enteric pharmaceutical formulations are manufactured in such a way that the product passes unchanged through the stomach of the patient, and dissolves and releases the active ingredient quickly when it leaves the stomach and enters the small intestine. Such formulations have long been used, and may be in capsule, tablet or pellet form. When in tablet or pellet form, the active ingredient is in the inner part of the tablet or pellet and is enclosed in a film or envelope, the “enteric coating”, which is insoluble in acid environments, such as the stomach, but is soluble in near-neutral environments such as the small intestine.

Disclosed herein are orally administrable, pharmaceutical compositions comprising the compounds disclosed herein. In one embodiment, the orally administrable, pharmaceutical compositions are enterically coated. The enterically coated compositions provide at least one of greater bioavailability or reduced stomach upset, when compared to orally administrable, non-enterically coated pharmaceutical compositions. The compositions disclosed herein may be administered to a patient in either a fasted or a fed state.

Disclosed herein are 1) enterically coated capsules, 2) enterically coated tablets, and 3) enterically coated pellets, wherein each of these comprises the compounds disclosed herein. In one embodiment, the compound is Formula I.

### Enterically Coated Capsules

Disclosed herein is a dosage form for oral administration. The dosage forms described herein are composed of a core composition that comprises a compound as disclosed herein (e.g., Formula I, Formula II), wherein the compound is contained in a capsule that is sealed and coated with an enteric coating.

In an embodiment, the enterically coated, orally administrable pharmaceutical composition is an enterically coated capsule that comprises: a) a core composition comprising a compound disclosed herein; b) a capsule that contains the core composition, wherein the capsule has a body and a cap; c) a polymeric seal covering the transition between the capsule cap and body; and d) an enteric coating that coats the polymeric seal and capsule.

The enterically coated capsules and tablets release little, if any of the compounds disclosed herein, in fluid having pH<4.5 (i.e., the stomach) and release the majority of the compound in fluid having pH>6.0 (i.e., the intestines). In an embodiment, the dosage form releases: none of the compound disclosed herein (e.g., Formula I, Formula II) for up to 2 hours in fluid having pH between 4.5 and 6.0; and the majority of the compound in fluid having pH>6.0.

In an embodiment, the enteric capsule may be Evonik (Eudracaps® (size 0)), Capsugel (Enprotect® (Size 0)), and CanadaCaps. In one embodiment, the enteric capsule is Eudracaps®. In one embodiment, the enteric capsule is Enprotect®. In one embodiment, the enteric capsule is CanadaCaps.

The enteric coating is made of at least one polymer. In an embodiment, the enteric coating comprises a copolymer. The copolymer may contain at least one of methyl acrylate, methyl methacrylate, methacrylic acid or ethyl acrylate.

According to another embodiment, the enteric coating comprises poly[methacrylic acid, ethyl acrylate] wherein the methacrylic acid and ethyl acrylate are present in a ratio of about 1:1.

According to another embodiment, the enteric coating comprises polymethyl acrylate, methyl methacrylate, methacrylic acid in a ratio of about 7:3:1.

In another embodiment, the core composition further comprises a permeation enhancer that is selected from the group consisting of sodium N-[8-(2-hydroxybenzoyl) amino] caprylate (SNAC), salcaprozate sodium, sodium caprate (CIO), or 8-(N-2-hydroxy-5-chlorobenzoyl)-amino-caprylic acid (5-CNAC).

According to another embodiment of the present disclosure there is provided a solid oral pharmaceutical dosage form comprising: a) a core composition comprising a compound as disclosed herein (e.g., Formula I, Formula II) and CIO; b) a capsule that contains the core composition and wherein the capsule has a body and a cap; c) a polymeric seal covering the transition between the capsule cap and body; and d) an enteric coating that coats the polymeric seal and capsule, wherein the coating comprises one or more co-polymers selected from the group consisting of: i) poly[methacrylic acid and ethyl acrylate], wherein the methacrylic acid and ethyl acrylate are present in a ratio of about 1:1; and ii) poly[methyl acrylate, methyl methacrylate, methacrylic acid], wherein the methyl acrylate, methyl methacrylate and methacrylic acid are present in a ratio of about 7:3:1.

In yet another embodiment of the present disclosure there is provided a method of treating a disease or condition in a patient in need thereof comprising administering a dosage form as described herein.

In still another embodiment, there is provided an enteric dosage form of the present disclosure for use in the treatment of a disease or condition in a patient.

According to another embodiment of the present disclosure there is provided a process for preparing a dosage form as described herein, comprising: a) blending all components comprised in the core composition in a suitable blender; b) weighing an amount of the blend formed in step (a) to be the core composition; c) compressing the blend weighed in step (b) using a capsule slug mold; d) placing the slug formed in step (c) into the capsule; e) covering the transition between the capsule cap and body with the polymeric seal; and f) coating the capsule with the enteric coating.

Oral dosage forms that release the disclosed compounds (e.g., Formula I, Formula II) in the middle or distal regions of the intestine provide at least one of higher bioavailability of the therapeutic agent as compared to those that release earlier in the digestive tract, such as the stomach, duodenum or proximal region of small intestine or decreased stomach upset, when compared to an immediate release formulation. Such targeted release may be achieved through the use of the dosage forms described herein.

The dosage forms described herein are able to achieve such targeted release regardless of whether they are administered to the patient in a fed or fasted state. The potential benefits of the availability of such flexible dosing timing and scheduling include improved adherence and efficacy.

Dosage forms designed to target different regions of the intestine were described previously. See, e.g., Maroni, Alessandra, et al. In vitro and in vivo evaluation of an oral multiple-unit formulation for colonic delivery of insulin. EUROPEAN JOURNAL OF PHARMACEUTICS AND BIOPHARMACEUTICS 108 (2016): 76-82; Schellekens, R. C. A., et al. Pulsatile drug delivery to ileo-colonic segments by structured incorporation of disintegrants in pH-responsive polymer coatings. JOURNAL OF CONTROLLED RELEASE 132.2 (2008): 91-98; Liu, Fang, et al. Evolution of a physiological pH 6.8 bicarbonate buffer system: application to the dissolution testing of enteric coated products. European Journal of Pharmaceutics and Biopharmaceutics 78.1 (2011): 151-157; Maroni, Alessandra, et al. In vitro and in vivo evaluation of an oral multiple-unit formulation for colonic delivery of insulin. EUROPEAN JOURNAL OF PHARMACEUTICS AND BIOPHARMACEUTICS 108 (2016): 76-82; Schellekens, R. C. A., et al. Pulsatile drug delivery to ileo-colonic segments by structured incorporation of disintegrants in pH-responsive polymer coatings. JOURNAL OF CONTROLLED RELEASE 132.2 (2008): 91-98; Liu, Fang, et al. Evolution of a physiological pH 6.8 bicarbonate buffer system: application to the dissolution testing of enteric coated products. EUROPEAN JOURNAL OF PHARMACEUTICS AND BIOPHARMACEUTICS 78.1 (2011): 151-157.

The enteric coatings that provide the targeted release characteristics described herein include coatings comprised of one or more polymers or co-polymers. Nonlimiting examples of such polymers or co-polymers include those identified in Table 1 below, where the listed trade names are commercially available products that comprise the polymers or co-polymers indicated, in many cases as aqueous dispersions:

TABLE 1

| | Polymer | |
|---|---|---|
| Chemical Name | Ratio | Example Trade name(s) |
| poly[butyl methacrylate, (2- | 1:2:1 | Eudragit ® E 100, Eudragit ® |
| dimethyaminoethyl) methacrylate, | | E12.5, |
| methyl methacrylate | | Eudragit ® E PO |
| Poly[ethyl acrylate, methyl | 2:1 | Eudragit ® NE 30 D, Eudragit ® |
| methacrylate] | | NE 40 D, Eudragit ® NM 30 D |
| Poly[methacrylic acid, methyl | 1:1 | Eudragit ® L 100, Eudragit ® L |
| methacrylate] | | 12.5, |
| | | Eudragit ® L 12.5 P |
| Poly[methacrylic acid, ethyl | 1:1 | Acryl-EZE, Acryl-EZE 93A, |
| acrylate] | | Acryl-EZE MP, Eudragit ® L30 D- |
| | | 55, Eudragit ® L 100-55, Eastacryl |
| | | 30 D, Kollicoat ® MAE 30 DP, |
| | | Kollicoat ® MAE 100 P |
| Poly[methacrylic acid, methyl | 1:2 | Eudragit ® S 100, Eudragit ® S |
| methacrylate] | | 12.5, Eudragit ® S 12.5 P |
| Poly[methyl acrylate, methyl | 7:3:1 | Eudragit ® FS30 D |
| methacrylate, methacrylic acid] | | |
| Poly[ethyl acrylate, methyl | 1:2:0.2 | Eudragit ® RL 100, Eudragit ® RL |
| methacrylate, trimethylammonioethyl | | PO, Eudragit ® RL 30D, Eudragit ® |
| methacrylate chloride] | | RL 12.5 |
| Poly[ethyl acrylate, methyl | 1:2:0.1 | Eudragit ® RS 100, Eudragit ® RS |
| methacrylate, trimethylammonioethyl | | PO, Eudragit ® RS 30 D, |
| methacrylate chloride] | | Eudragit ® RS 12.5 |
| HPMCAS (H) | NA | |
| Hydroxyl propyl methyl cellulose | NA | |
| phthalate (HPMCP) | | |
| Polyvinyl acetate phthalate (PVAP) | NA | |
| Cellulose acetate trimellitate (CAT) | NA | |
| Cellulose acetate phthalate (CAP) | NA | |

In certain embodiments, coatings that may be used in dosage forms of the present disclosure are comprised of one or more of Poly [methacrylic acid, ethyl acrylate] (1:1 ratio) (e.g., Eudragit® L30D-55), Poly [methyl acrylate, methyl methacrylate, methacrylic acid] (7:3:1 ratio) (e.g., Eudragit® F S30D), Poly [methacrylic acid, methyl methacrylate] (1:2 ratio) (e.g., Eudragit® S), Poly[methacrylic acid, methyl methacrylate] (1:1 ratio) (e.g., Eudragit® L) and HPMCAS (H). Examples of specific co-polymers include Poly[methacrylic acid, ethyl acrylate] (1:1 ratio) (e.g., Eudragit® L30D-55), Poly [methyl acrylate, methyl methacrylate, methacrylic acid] (7:3:1 ratio) (e.g., Eudragit® FS30D) and mixtures thereof.

In some embodiments, the coating comprises include between 10 and 70% Poly [methyl acrylate, methyl methacrylate, methacrylic acid] (7:3:1 ratio) (e.g., Eudragit® FS30D). In certain embodiments the coating comprises about 51.6% Poly [methyl acrylate, methyl methacrylate, methacrylic acid] (7:3:1 ratio) (e.g., Eudragit® FS30D). In certain embodiments the coating comprises about 53.3% Poly [methyl acrylate, methyl methacrylate, methacrylic acid] (7:3:1 ratio) (e.g., Eudragit® FS30D).

In certain embodiments the coating comprises a combination of Poly[methacrylic acid, ethyl acrylate] (1:1 ratio) (e.g., Eudragit® L30D-55), Poly[methyl acrylate, methyl methacrylate, methacrylic acid] (7:3:1 ratio) (e.g., Eudragit® FS30D). In certain embodiments the coating comprises a combination of about 13.3 percent Poly[methacrylic acid, ethyl acrylate] (1:1 ratio) (e.g., Eudragit® L30D-55) and about 53.3 percent Poly [methyl acrylate, methyl methacrylate, methacrylic acid] (7:3:1 ratio) (e.g., Eudragit® FS30D). In certain embodiments the coating comprises a combination of about 12.9 percent Poly[methacrylic acid, ethyl acrylate] (1:1 ratio) (e.g., Eudragit® L30D-55) and about 51.6 percent Poly [methyl acrylate, methyl methacrylate, methacrylic acid] (7:3:1 ratio) (e.g., Eudragit® F S30D).

In addition to the composition of the coating, the release profile of the dosage form is also influenced by the amount of coating applied to the sealed capsule, referred to herein as the “coating level.” This dimension is expressed herein as the mass of coating per unit area of the capsule, most typically mg/cm² in the context of the dosage forms described herein. In certain embodiments, the coating level is between 5-20 mg/cm². In certain embodiments, the coating level is between 6-16 mg/cm². In certain embodiments, the coating level is between 7-10 mg/cm². In certain embodiments, the coating level is about 6, about 7, about 8, about 9, about 10, about 11, about 12, about 13, about 14, about 15 or about 16 mg/cm². In certain embodiments the coating level is about 6.5, about 7.5, about 8.5, about 9.5, about 10.2, about 10.5, about 10.9, about 11.9, about 12.9, about 13.0 or about 13.7 mg/cm².

The polymer(s) and co-polymer(s) described above are typically provided in the form of aqueous dispersions. A significant component of the enteric coatings described herein therefore is comprised of purified water. In certain embodiments, the water content is between 20-40 percent. In certain embodiments, the water content is about 33 percent.

In some embodiments, the coating includes other excipients that improve the manufacturability and/or function of the coating. An example of such an excipient that may be included is a plasticizer. Commonly used plasticizers include Propylene glycol, Glycerol, Polyethylene glycols (e.g., PEG-400), Glyceryl triacetate (Triacetin), Triethyl citrate (TEC), Acetyl triethyl citrate, Phthalate esters, Diethyl phthalate, Acetylated, Castor oil and Mineral oil. In certain embodiments, the coating comprises TEC. In certain embodiments, the concentration of TEC is from 1-10 percent. In certain embodiments the TEC content is about 3%.

Another excipient that may be included is a pH modifier, which may be used to adjust the pH at which the polymer(s) or co-polymer(s) dissolves. Examples of such pH modifiers are known by those skilled in the art, and include sodium carbonate, sodium bicarbonate, potassium dihydrogen phosphate and ammonium hydroxide.

Another component that may be added as an excipient in the coating composition and/or added to the outside of the coating is a material used to prevent tackiness during storage. An example of such a material is talc.

In addition to the effects of the coatings described above, the release profiles of the dosage forms described herein also result from the use of a sealed capsule. The capsules themselves are hard shelled capsules known in the art, commonly made from natural materials such as gelatin, polysaccharide derivatives, such as carrageenans, or celluloses, such as methylcellulose or hydroxypropyl methylcellulose (HPMC). Capsules are comprised of two pieces: a capsule body that is filled with a core composition and a cap that fits over the body. Selection of a particular capsule for use in the dosage forms described herein is within the skill of persons skilled in the art, and will depend in part on the volume of the core composition to be contained therein. In certain embodiments the capsule is comprised of HPMC.

Capsules are typically soluble in gastrointestinal fluids, so in order to avoid release upstream of the region of the intestine at which release is desired, the capsule must be protected from contact with gastrointestinal fluids before reaching that point. Such protection is provided by using a suitable enteric coating as described above, but in order for the enteric coating to provide such protection, it must coat the entirety of the capsule, i.e., with no gaps through which fluid may ingress.

It has been found that the addition of a polymeric seal over the edge of the cap of the capsule, where the cap transitions to the body of the capsule, prior to application of the enteric coating helps avoid release in the proximal small intestine and contributes to improved bioavailability. The amount of surface area of the capsule covered by the seal is not critical, as long as it covers the transition from the cap to the body. Thus, the seal may partially or wholly cover the capsule. For example, in certain embodiments the seal may be a thin band around the capsule, and in other embodiments may be a coat or subcoat that covers the entirety of the capsule.

In some embodiments, the polymeric seal is provided through a process known as banding. Banding is typically used on capsule dosage forms filled with liquids to prevent leakage of the liquid interiors, but in the context of the present disclosure, banding serves to prevent external liquid from reaching the capsule's core composition until the enteric coating has dissolved, as noted above.

Seals for use in dosage form of the present compositions, including bands, may be made from materials having the same dissolution properties as the enteric coating itself, or may have no enteric properties. Seals, including bands, may therefore be made from materials similar to those described above for either the enteric coating or from the same material as the capsule itself. In certain embodiments the band is comprised of the same composition as the enteric coating. In certain embodiments the band is comprised of HPMC. In certain embodiments the band comprises HPMC, ethanol and water. In certain embodiments the band comprises about 17% HPMC, about 58% ethanol and 25% water.

Permeation enhancers increase local permeability (Twarog et al. 2019) and result in increased oral bioavailability for therapeutic use. Examples of permeation enhancers that may be used in the formulations disclosed herein include sodium N-[8-(2-hydroxybenzoyl) amino] caprylate (SNAC), sodium caprylate (C8), sodium caprate or sodium decanoate (CIO), or 8-(N-2-hydroxy-5-chlorobenzoyl)-amino-caprylic acid (5-CNAC). Salcaprozate sodium has generally regarded as safe (GRAS) status and is contained in FDA-approved medical food (Eligen®-Vitamin B1 2, Emisphere, Roseland, NJ, USA). Rybelsus® tablet, which is approved by FDA, European Medicines Agency and Japan Pharmaceuticals and Medical Devices Agency, contains SNAC at 300 mg. CIO has food additive status with no daily limits on consumption.

The core compositions of the present disclosure may contain a capsule filler to improve at least one of flow, compactability, tackiness, and density of the blend, in the core. An example of such a material is microcrystalline cellulose (MCC). In certain embodiments, dosage forms of the present disclosure comprise MCC. The core compositions disclosed herein may comprise up to about 80 mg MCC. In certain embodiments, core compositions of the present disclosure comprise no more than about 60 mg MCC.

The composition may include additional functional excipients, such as common tableting/encapsulation excipients. Examples include a lubricant (e.g., magnesium stearate, sodium stearyl fumarate (SFF)), disintegrant (sodium starch glycolate, crospovidone, croscarmellose sodium, starch, etc.) and/or a glidant (colloidal silica, starch, silicone oil, talc, etc.).

An advantage of the dosage forms described herein is that they may achieve acceptable bioavailability regardless of whether they are administered while the patient is in a fed state or a fasted state. When used herein, the term “fed state,” also known as the absorptive state, refers to the condition of a person's body after they have eaten food and the body is digesting the food and absorbing the nutrients. This condition begins when food is eaten, and may remain for up to about 4 hours depending on the types and amounts of nutrients ingested. The term “fasted state,” by contrast refers to the condition of a person's body when it is not digesting food and absorbing nutrients. As the duration of the fed state varies, drugs requiring administration during a fasted state are commonly required to be taken in the morning, before breaking the nighttime fast. In certain embodiments, negative food effects on bioavailability are not observed when dosage forms of the present disclosure are co-administered with food. Dosage forms of the present disclosure may be used in the treatment of a range of diseases or disorders.

The dosage forms described herein may also (or alternatively) reduce stomach upset that may be experienced by the patient, compared to when the patient takes a non-enterically coated dosage form.

When used in the treatment of diseases or conditions such as cancer, more specifically, such as cancers susceptible to treatment with a KRAS G12D inhibitor. Examples of such cancers include pancreatic cancer, colorectal cancer and non-small cell lung cancer (NSCLC). The dosage forms disclosed herein may be administered once daily, twice daily, alternate days, every third day, every fourth day, every fifth day, every sixth day or once weekly. Preferably, the dosage forms are administered once daily.

When used herein, the terms “treatment,” “treat,” “treating,” and the like, are meant to include slowing or attenuating the progression of a disease or disorder. These terms also include alleviating, ameliorating, attenuating, eliminating, or reducing one or more symptoms of a disorder or condition, even if the disorder or condition is not actually eliminated and even if progression of the disorder or condition is not itself slowed or reversed.

As used herein, the term “about” is intended to refer to an acceptable degree of error for the amount or quantity indicated given the nature or precision of the measurements. For example, the degree of error can be indicated by the number of significant figures provided for the measurement, as is understood in the art, and includes but is not limited to a variation of +/−1 in the most precise significant figure reported for the amount or quantity. Typical exemplary degrees of error are within 20 percent (%), preferably within 10%, and more preferably within 5% of a given value or range of values. For instance, “about” may mean the numeric value may be modified by ±5%, ±4%, ±3%, ±2%, ±1%, ±0.5%, ±0.4%, ±0.3%, ±0.2%, ±0.1% or ±0.05%. Numeric values modified by the term “about” include the specific identified value. For example, “about 5.0” includes 5.0.

### Preparing Enterically Coated Capsules

The enterically coated capsules may be prepared by blending a compound disclosed herein, such as Formula I, and CIO in a suitable blender (at small-scale, all the components are weighed and transferred into ajar and blended for 5 mins at 25 G in a resonant acoustic mixer), weighing the target blend, compressing the blend into a slug using an appropriate capsule slug mold, and placing it to fit inside an enteric capsule body.

The filled capsules are then coated using a pan to different levels of coating expressed in mg/cm². After coating, each capsule is manually sealed using 10 uL coating solution around cap and body transition area, allowed to dry and then stored refrigerated. The blending conditions, encapsulation/coating/sealing processes are further modified upon scale-up to achieve optimum manufacturability and required capsule attributes depending upon the scale and manufacturing equipment utilized.

### Enteric Tablet

The compounds disclosed herein (e.g., Formula I) may be formulated as an enteric tablet. In one embodiment, the tablet comprises a compound as disclosed herein, such as Formula I, microcrystalline cellulose, croscarmellose sodium, silicon dioxide, hydroxypropyl methyl cellulose, and magnesium stearate. In one embodiment, the enteric tablet contains about 30 wt % to about 50 wt % of a compound disclosed herein. In one embodiment, the tablets further contain titanium dioxide, talc, glyceryl mono and dicaprylcaprate, polyethylene glycol-polyvinyl alcohol graft copolymer, polyvinyl alcohol, and optionally, a coloring agent. The enteric layer is discussed below.

### Enteric Pellet

The compounds disclosed herein may be formulated as an enteric pellet, which is then optionally put into a capsule. The capsule may be enterically coated or not. Thus, in one embodiment, the enterically coated pellets are contained within a non-enterically coated capsule, such as a gelatin capsule.

The enteric pellets comprise a) a core consisting of a compound as disclosed herein and one or more pharmaceutically acceptable excipients; b) an optional separating layer; c) an enteric layer comprising hydroxypropyl-methylcellulose acetate succinate (HPMCAS) and a pharmaceutically acceptable excipient; d) an optional finishing layer. In one embodiment, the compound is Formula I.

Also disclosed herein is a method of manufacturing an enteric pellet comprising a) providing a core consisting of a compound as disclosed herein and one or more pharmaceutically acceptable excipients; b) optionally, applying to the core a separating layer comprising one or more pharmaceutically acceptable excipients; c) applying an enteric layer comprising HPMCAS and one or more pharmaceutically acceptable excipients, wherein the HPMCAS is applied as an aqueous solution or suspension and the application takes place in an apparatus of the fluid bed type; d) optionally, applying a finishing layer.

### The Core of the Pellet

A core for the pellet is prepared by applying a layer comprising the compounds as disclosed herein (e.g., Formula I), to an inert core. Such inert cores are conventionally used in pharmaceutical science, and are readily purchased in all industrial countries. In some embodiments, the core is prepared from starch and sucrose, for use in confectionery as well as in pharmaceutical manufacturing. However, cores of any pharmaceutically acceptable excipient may be used, including, for example, microcrystalline cellulose, vegetable gums, waxes, and the like. The primary characteristic of the inert core is to be inert, with regard both to the compounds disclosed herein and the other excipients in the pellet, and with regard to the patient who will ultimately ingest the pellet.

The size of the cores depends, for example, on the desired size of the pellet to be manufactured. In general, pellets can be as small as 0.1 mm, or as large as 2 mm. cores are from about 0.3 to about 0.8 mm, in order to provide finished pellets in the desired size range of from about 0.5 to about 1.5 mm in diameter.

In some embodiments, the cores may be specified as being of particle size ranges such as from 18 to 20 U.S. mesh, from 20 to 25 U.S. mesh, from 25 to 30 U.S. mesh, or from 30 to 35 U.S. mesh to obtain acceptable size distributions of various absolute sizes.

The amount of cores to be used depends on the weights and thicknesses of the added layers; in general, the cores comprise from about 10 to about 70 percent of the product. More preferably, the charge of cores represents from about 15 to about 45 percent of the product.

When manufacture of the pellet begins with inert cores, the compound is coated on the cores to yield a final drug concentration of about 10 to about 25 percent of the product, in general. The amount of compound, of course, depends on the desired dose of the drug and the quantity of pellets which it is desired to administer.

A convenient manner of coating the cores with compound is the “powder coating” process where the cores are moistened with a sticky liquid or binder, compound is added as a powder, and the mixture is dried. Such a process is regularly carried out in the practice of industrial pharmacy, and suitable equipment is in daily use. Such equipment is, in fact, used in several steps of the present process, and it will, accordingly, be discussed here. Historically, this process has been conducted in conventional coating pans similar to those employed in sugar coating processes. This process can be used to prepare pellets, but this equipment has less efficient air flow and drying capabilities which limits application rates and can result in longer processing times in order to minimize agglomerations.

Alternately, the present product could be made in fluidized bed equipment (using a rotary processor), or in rotating plate equipment such as the Freund CF-Granulator (Vector Corporation, Marion, Iowa). The rotating plate equipment typically consists of a cylinder, the bottom of which is a rotatable plate. Motion of the mass of particles to be coated is provided by friction of the mass between the stationary wall of the cylinder and the rotating bottom of it. Means can be provided to apply warm air to dry the mass, and liquids can be sprayed on the mass and balanced against the drying rate as in the fluidized bed case.

When a powder coating is to be applied, the mass of pellets, in the present case, is maintained in a sticky state, and the powder to be adhered to them, compound in this case, is added continuously or periodically and adheres to the sticky pellets. When all of the compound has been applied, the spray is stopped and the mass is allowed to dry in the air stream. It may be appropriate or convenient to add some inert powders to the compound.

Additional solids may be added to the layer with compound. These solids may be added to facilitate the coating process as needed to aid flow, reduce static charge, aid bulk buildup and form a smooth surface. Inert substances such as talc, kaolin, and titanium dioxide, lubricants such as magnesium stearate, finely divided silicon dioxide, crospovidone, and non-reducing sugars, e.g., sucrose, may be used. The amounts of such substances are in the range from about a few tenths of 1% of the product, up to about 20% of the product. Such solids should be of fine particle size, less than 50 μm, to produce a smooth surface.

The compound is made to adhere to the cores by spraying a pharmaceutical excipient which is sticky and adherent when it is wet, and dries to a strong, coherent film. Pharmaceutical scientists are aware of and conventionally use many such substances, most of them polymers. such polymers include hydroxypropylmethylcellulose, hydroxypropyl-cellulose and polyvinylpyrrolidone. Additional such substances include methylcellulose, carboxymethylcellulose, acacia and gelatin, for example. The amount of the adhering excipient is in the range from about 4% to about 12% of the product, and depends in large part on the amount of compound to be adhered to the core.

Compound may also be built up on the cores by spraying a slurry comprising compound suspended in a solution of the excipients of the compound layer, dissolved or suspended in sufficient water to make the slurry sprayable. Such a slurry may be milled through a machine adapted for grinding suspensions in order to reduce the particle size of compound. Grinding in suspension form is desirable because it avoids dust generation and containment problems which arise in grinding dry powder drugs. A method for applying this suspension is in the classic pharmaceutical fluidized bed coating device, such as the Wurster column, which consists simply of a vertical cylinder with an air-permeable bottom and an upward spraying nozzle close above the bottom, or a downward-spraying nozzle mounted above the product mass. The cylinder is charged with particles to be coated, sufficient volume of air is drawn through the bottom of the cylinder to suspend the mass of particles, and the liquid to be applied is sprayed onto the mass. The temperature of the fluidizing air is balanced against the spray rate to maintain the mass of pellets or tablets at the desired level of moisture and stickiness while the coating is built up.

On the other hand, the core may comprise a monolithic particle in which the compound is incorporated. Such cores may be prepared by the granulation techniques which are wide spread in pharmaceutical science, particularly in the preparation of granular material for compressed tablets. The particle size of the cores is too small for preparation by compression techniques, but the cores may be prepared by mixing the compound into a mass of pharmaceutical excipients, moistening the mass with water or a solvent, drying, and breaking the mass into sized particles in the same size range as described above for the inert cores. This can be accomplished via the process of extrusion and marumerization.

The core for the pellet can also be prepared by mixing compound with conventional pharmaceutical ingredients to obtain the desired concentration and forming the mixture into cores of the desired size by conventional procedures or by the process.

### Separating Layer

The separating layer between the compound-containing core and the enteric layer is not required, but is a feature of the formulation. The separating layer, if required, provides a smooth base for the application of the enteric layer, to prolong the pellet's resistance to acid conditions, and to improve stability by inhibiting any interaction between the drug and the enteric polymer in the enteric layer.

The smoothing function of the separating layer is purely mechanical, the objective of which is to improve the coverage of the enteric layer and to avoid thin spots in it, caused by bumps and irregularities on the core. Accordingly, the more smooth and free of irregularities the core can be made, the less material is needed in the separating layer, and the need for the smoothing characteristic of the separating layer may be avoided entirely when the compound is of extremely fine particle size and the core is made as close as possible to truly spherical.

In general, the separating layer is composed of coherent or polymeric materials, and finely powdered solid excipients which constitute fillers. When a sugar is used in the separating layer, it is applied in the form of an aqueous solution and constitutes part of or the whole of the coherent material which sticks the separating layer together. In addition to or instead of the sugar, a polymeric material may also be used in the separating layer. For example, substances such as hydroxypropylmethylcellulose, polyvinylpyrrolidone, hydroxypropylcellulose and the like may be used in small amounts to increase the adherence and coherence of the separating layer.

It is further advisable to use a filler excipient in the separating layer to increase the smoothness and solidity of the layer. Substances such as finely powdered talc, silicon dioxide and the like are universally accepted as pharmaceutical excipients and may be added as is convenient in the circumstances to fill and smooth the separating layer.

In general, the amount of sugar in the separating layer may be in the range of from about 2% to about 10% of the product, when a sugar is used at all, and the amount of polymeric or other sticky material may be in the range of from about 0.1 to about 5%. The amount of filler, such as talc, should be in the range of from about 5 to about 15%, based on final product weight.

The separating layer may be applied by spraying aqueous solutions of the sugar or polymeric material, and dusting in the filler as has been described in the preparation of a compound layer. The smoothness and homogeneity of the separating layer can be improved, however, if the filler is thoroughly dispersed as a suspension in the solution of sugar and/or polymeric material, and the suspension is sprayed on the core and dried, using equipment as described above in the preparation of cores with compound layers.

### Enteric Layer

The enteric layer is comprised of an enteric polymer, which must be chosen for compatibility with compound as discussed above. The polymer must be one having only a small number of carboxylic acid groups per unit weight or repeating unit of the polymer. In one embodiment, the enteric polymer is hydroxypropylmethylcellulose acetate succinate (HPMCAS), which product is defined as containing not less than 4% and not more than 28% of succinoyl groups, which are the only free carboxylic groups in the compound. See Japanese Standards of Pharmaceutical Ingredients 1991, page 1216-21, Standard No. 19026. HPMCAS is available from Shin-Etsu Chemical Co., Ltd., Tokyo, Japan, under the trademark AQOAT. It is available in two particle size grades and three molecular weight ranges. The L grade, having number average molecular weight of 93,000, is used in the present examples but other grades are expected to be usable.

Enteric polymers may be applied as coatings from aqueous suspensions, from solutions in aqueous or organic solvents, or as a powder. Application from organic solvents is presently not at all favored in the pharmaceutical industry, because of the cost of the solvent and the difficulty in either disposing of solvent vapors or recovering the evaporated solvent. Accordingly, no detailed discussion of application of the enteric layer from organic solvents will be given here, but the pharmaceutical scientist will recognize that such application is entirely possible if circumstances favor it.

The enteric polymer can also be applied according to a method described by Shin-Etsu Chemical Co. Ltd. (Obara, et al., Poster PT6115, AAPS Annual Meeting, Seattle, Wash., Oct. 27-31, 1996). When the enteric polymer is applied as a powder the enteric polymer is added directly in the solid state to the tablets or pellets while plasticizer is sprayed onto the tablets or pellets simultaneously. The deposit of solid enteric particles is then turned into a film by curing. The curing is done by spraying the coated tablets or pellets with a small amount of water and then heating the tablets or pellets for a short time. This method of enteric coating application can be performed employing the same type of equipment as described above in the preparation of cores with compound layers.

When the enteric polymer is applied as an aqueous suspension, a problem in obtaining a uniform, coherent film often results. It is very advisable, accordingly, to purchase a fine particle grade or grind the particles of polymer to an extremely small size before application. It is possible either to grind the dry polymer, as in an air-impaction mill or to prepare the suspension and grind the polymer in slurry form. Slurry grinding is generally preferable, particularly since it can be used also to grind the filler portion of the enteric layer in the same step. It is advisable to reduce the average particle size of the enteric polymer to the range from about 1 μm to about 5 μm, preferably no larger than 3 μm.

When the enteric polymer is applied in the form of a suspension, it is important to assure that the suspension remains homogeneous, and that conditions which favor the agglomeration of the polymer do not occur. Such precautions include maintaining the suspension in a gently stirred condition, but not stirring so vigorously as to create foam, and assuring that the suspension does not stand still in eddies in nozzle bodies, for example, or in over-large delivery tubing. Frequently polymers in suspension form will agglomerate if the suspension becomes too warm, and the critical temperature may be as low as 30° C. in individual cases. Since spray nozzles and tubing are exposed to hot air in the usual fluid bed type equipment, care must be taken to assure that the suspension is kept moving briskly through the equipment to cool the tubing and nozzle. When HPMCAS is used, in particular, it is advisable to cool the suspension below 20° C. before application, to cool the tubing and nozzle by pumping a little cold water through them before beginning to pump the suspension, and to use supply tubing with as small a diameter as the spray rate will allow so that the suspension can be kept moving rapidly in the tubing.

It is in the present disclosure, however, to apply the enteric polymer as an aqueous solution whenever it is possible to do so. In the case of HPMCAS, dissolution of the polymer can be obtained by neutralizing the polymer, preferably with ammonia. Neutralization of the polymer may be obtained merely by adding ammonia, preferably in the form of aqueous ammonium hydroxide to a suspension of the polymer in water; complete neutralization results in complete dissolution of the polymer at about pH 5.7-5.9. Good results are also obtained when the polymer is partially neutralized, by adding less than the equivalent amount of ammonia. In such case, the polymer which has not been neutralized remains in suspended form, suspended in a solution of neutralized polymer. As noted earlier, it is obviously important to control the particle size of the polymer when such a process is to be used. Use of neutralized polymer more readily provides a smooth, coherent enteric layer than when a suspended polymer is used, and use of partially neutralized polymer provides intermediate degrees of smoothness and coherency. Particularly when the enteric layer is applied over a very smooth separating layer, excellent results may be obtained from partially neutralized enteric polymer.

The extent of neutralization may be varied over a range without adversely affecting results or ease of operation. For example, operation with from about 25% to about 100% neutralization is in the present disclosure. Another condition is from about 45% to about 100% neutralization, and another condition is from about 65% to about 100%. Still another manner of neutralization is from about 25% to about 65% neutralized. It is found, however, that the enteric polymer in the resulting product, after drying, is neutralized to a lesser extent than when applied. When neutralized or partially neutralized HPMCAS is applied, the HPMCAS in the final product is from about 0% to about 25% neutralized, more preferably from about 0% to about 15% neutralized.

Most enteric polymers require the addition of a plasticizer for best results. In the case of HPMCAS, the plasticizer is triethyl citrate, used in an amount up to about 15%-30% of the amount of enteric polymer in aqueous suspension application. When a neutralized HPMCAS is employed, lower levels or no plasticizer may be required.

Minor ingredients, such as antifoam, suspending agents when the polymer is in suspended form, and surfactants to assist in smoothing the film are also commonly used. For example, silicone anti-foams, surfactants such as polysorbate 80, sodium lauryl sulfate and the like and suspending agents such as carboxymethylcellulose, vegetable gums and the like may commonly be used at amounts in the general range up to 1% of the product.

Usually, an enteric layer is filled with a powdered excipient such as talc, glyceryl monostearate or hydrated silicon dioxide to build up the thickness of the layer, to strengthen it, to reduce static charge, and to reduce particle cohesion. Amounts of such solids in the range of from about 1% to about 10% of the final product may be added to the enteric polymer mixture, while the amount of enteric polymer itself is usually in the range from about 5% to about 25%, more preferably, from about 10% to about 20%.

Application of the enteric layer to the pellets follows the same general procedure previously discussed, using fluid bed type equipment with simultaneous spraying of enteric polymer solution or suspension and warm air drying. Temperature of the drying air and the temperature of the circulating mass of pellets should be kept in the ranges advised by the manufacturer of the enteric polymer.

### Finishing Layer

A finishing layer over the enteric layer is not always necessary, but it frequently improves the elegance of the product and its handling, storage and machinability and may provide further benefits as well. The simplest finishing layer is simply a small amount, about less than 1% of an anti-static ingredient such as talc or silicon dioxide, simply dusted on the surface of the pellets. Another simple finishing layer is a small amount, about 1%, of a wax such as beeswax melted onto the circulating mass of pellets to further smooth the pellets, reduce static charge, prevent any tendency for pellets to stick together, and increase the hydrophobicity of the surface.

More complex finishing layers may constitute a final sprayed-on layer of ingredients. For example, a thin layer of polymeric material such as hydroxypropylmethylcellulose, polyvinylpyrrolidone and the like, in an amount such as from about 2% up to about 10%, may be applied. The polymeric material may also carry a suspension of an opacifier, a bulking agent such as talc, or a coloring material, particularly an opaque finely divided color agent such as red or yellow iron oxide. Such a layer quickly dissolves away in the stomach, leaving the enteric layer to protect the compound, but provides an added measure of pharmaceutical elegance and protection from mechanical damage to the product.

Finishing layers to be applied to the present product are of essentially the same types commonly used in pharmaceutical science to smooth, seal and color enteric products, and may be formulated and applied in the usual manners.

### EMBODIMENTS

In an embodiment, the compound of Formula I is for use in therapy.

In another embodiment, the compound of Formula I is for use in the treatment of cancer.

In an embodiment, the compound of Formula I is for use in the treatment of a KRAS G12D associated cancer.

In an embodiment, examples of cancers that can be treated using the compounds disclosed herein (e.g., Formula I) include, but are not limited to lung cancer, pancreatic cancer, cervical cancer, esophageal cancer, endometrial cancer, ovarian cancer, cholangiocarcinoma, gastric cancer, and colorectal cancer. In some embodiments, the cancer comprises lung cancer, pancreatic cancer, cervical cancer, esophageal cancer, endometrial cancer, ovarian cancer, cholangiocarcinoma, or colorectal cancer.

In an embodiment, the compounds disclosed herein (e.g., Formula I) are for use in simultaneous, separate, or sequential combination with one or more of a PD-1 or PD-L1 inhibitor, a CDK4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof, in the treatment of cancer. An example of a PD-1 inhibitor is pembrolizumab. An example of a CDK4/CDK6 inhibitor is abemaciclib. An example of an EGFR inhibitor is cetuximab. In one embodiment, the compound of Formula I is administered in simultaneous, separate, or sequential combination with an effective amount of PD-1 inhibitor. In one embodiment, the compound of Formula I is administered in simultaneous, separate, or sequential combination with an effective amount of PD-1 inhibitor that comprises pembrolizumab. In one embodiment, the compound of Formula I is administered in simultaneous, separate, or sequential combination with an effective amount of a PD-L1 inhibitor. In one embodiment, the compound of Formula I is administered in simultaneous, separate, or sequential combination with an effective amount of a CDK4/CDK6 inhibitor. In one embodiment, the compound of Formula I is administered in simultaneous, separate, or sequential combination with an effective amount of a CDK4/CDK6 inhibitor that comprises abemaciclib. In one embodiment, the compound of Formula I is administered in simultaneous, separate, or sequential combination with an effective amount of an EGFR inhibitor. In one embodiment, the compound of Formula I is administered in simultaneous, separate, or sequential combination with an effective amount of an EGFR inhibitor that comprises cetuximab. In one embodiment, the compound of Formula I is administered in simultaneous, separate, or sequential combination with an effective amount of an ERK inhibitor. In one embodiment, the compound of Formula I is administered in simultaneous, separate, or sequential combination with an effective amount of an Aurora A inhibitor. In one embodiment, the compound of Formula I is administered in simultaneous, separate, or sequential combination with an effective amount of a SHP2 inhibitor. In one embodiment, the compound of Formula I is administered in simultaneous, separate, or sequential combination with an effective amount of a platinum agent. In one embodiment, the compound of Formula I is administered in simultaneous, separate, or sequential combination with an effective amount of pemetrexed.

In an embodiment according to any one of the “use” embodiments immediately above, a crystalline form of the compound disclosed herein is used. In some embodiments, the compound is Formula I. In a further embodiment, the crystalline form of a compound of Formula I is characterized by having an X-ray powder diffraction (XRPD) pattern comprising peaks at 2θ values of 4.4±0.2 and 11.3±0.2. The crystalline form may be further characterized by comprising peaks at 2θ values of 12.9±0.2 and 16.2±0.2. The crystalline form may still further be characterized by comprising peaks at 2θ values of 4.4±0.2, and 11.3, ±0.2, 17.5±0.2, 20.1±0.2, and 20.8±0.2 or 4.4±0.2, 11.3, ±0.2, 12.9±0.2, 16.2±0.2, 17.5±0.2, 20.1 0.2, and 20.8±0.2.

### Further Embodiments

The following are further embodiments of the present disclosure:
1. A saccharinate salt compound of

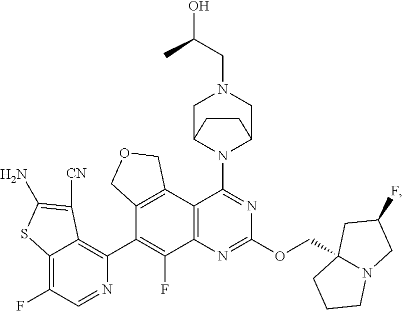 [SMILES: C[C@@H](O)CN1CC2CCC(C1)N2c1nc(OC[C@@]23CCCN2C[C@H](F)C3)nc2c(F)c(-c3ncc(F)c4sc(N)c(C#N)c34)c3c(c12)COC3]

wherein solvent may be optionally present, preferably the solvent is selected from water, tetrahydrofuran (THF), acetone, methanol, ethanol, and benzyl alcohol.
2. A compound of the formula:

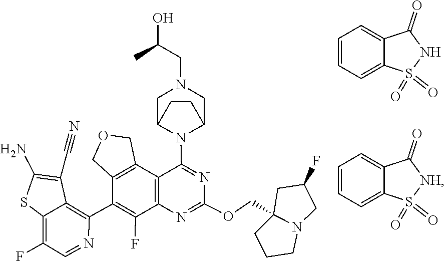

wherein solvent may be optionally present, preferably the solvent is selected from water, tetrahydrofuran (THF), acetone, methanol, ethanol, and benzyl alcohol.
3. The crystalline form of a compound of embodiment 1 or 2, wherein the crystalline form is characterized by having an X-ray powder diffraction (XRPD) pattern comprising peaks at 2θ values of 4.4±0.2 and 11.3±0.2.
4. The crystalline form according to embodiment 3, wherein the crystalline form is characterized by having an X-ray powder diffraction (XRPD) pattern further comprising peaks at 2θ values of 12.9±0.2 and 16.2±0.2.
5. The crystalline form according to embodiment 3 or 4, wherein the crystalline form is characterized by having an X-ray powder diffraction (XRPD) pattern further comprising peaks at 2θ values of 17.5±0.2, 20.1±0.2, and 20.8±0.2.
6. A pharmaceutical composition comprising a compound or crystalline form of any one of embodiments 1-5, and at least one excipient.
7. A method of treating a patient for cancer, comprising administering to a patient in need thereof, an effective amount of a pharmaceutical composition according to embodiment 6.
8. The method according to embodiment 7, wherein the cancer is a KRAS G12D associated cancer.
9. The method according to embodiment 7 or 8, wherein the cancer comprises lung cancer, pancreatic cancer, cervical cancer, esophageal cancer, endometrial cancer, ovarian cancer, cholangiocarcinoma, or colorectal cancer.
10. A compound or crystalline form according to any one of embodiments 1-5 for use in therapy.
11. A compound or crystalline form according to any one of embodiments 1-5 for use in the treatment of cancer.
12. The compound or crystalline form for use according to embodiment 11, wherein the cancer is a KRAS G12D associated cancer.
13. The compound or crystalline form for use according to embodiments 11 or 12, wherein the cancer is selected from lung cancer, pancreatic cancer, cervical cancer, esophageal cancer, endometrial cancer, ovarian cancer, cholangiocarcinoma, and colorectal cancer.
14. The compound or crystalline form for use according to embodiment 12, wherein the cancer is selected from colorectal cancer, pancreatic cancer, and gastric cancer.
15. A compound or crystalline form of any one of embodiments 1-5 for use in simultaneous, separate, or sequential combination with a PD-1 inhibitor in the treatment of cancer.
16. The compound or crystalline form for use according to embodiment 15, wherein the cancer is a KRAS G12D associated cancer.
17. The compound or crystalline form for use according to embodiments 15 or 16, wherein the cancer is selected from lung cancer, pancreatic cancer, cervical cancer, esophageal cancer, endometrial cancer, ovarian cancer, cholangiocarcinoma, and colorectal cancer.
18. The compound or crystalline form for use according to embodiments 16 or 17, wherein the cancer is selected from colorectal cancer, pancreatic cancer, and non-small cell lung cancer.
19. A compound or crystalline form of any one of embodiments 1-5 for use in simultaneous, separate, or sequential combination with a PD-1 inhibitor in the treatment of cancer, wherein the cancer is selected from colorectal cancer, pancreatic cancer, and gastric cancer.
20. The compound or crystalline form for use according to any one of embodiments 15-19, wherein the PD-1 inhibitor comprises pembrolizumab.
21. A compound or crystalline form of any one of embodiments 1-5 for use in simultaneous, separate, or sequential combination with an EGFR inhibitor in the treatment of cancer.
22. The compound or crystalline form of embodiment 21, wherein the cancer is a KRAS G12D associated cancer.
23. The compound or crystalline form of embodiments 21 or 22, wherein the cancer is selected from lung cancer, pancreatic cancer, cervical cancer, esophageal cancer, endometrial cancer, ovarian cancer, cholangiocarcinoma, and colorectal cancer.
24. The compound or crystalline form of embodiment 23, wherein the cancer is selected from colorectal cancer, pancreatic cancer, and non-small cell lung cancer.
25. The compound or crystalline form of embodiments 21, wherein the cancer is selected from colorectal cancer, pancreatic cancer, and gastric cancer.
26. The compound or crystalline form for use according to any one of embodiments 21-25, wherein the EGFR inhibitor comprises cetuximab.
27. A method of preparing a compound or crystalline form of any one of embodiments 1-5, the method comprising suspending 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile in a solvent, adding saccharin and isolating the disaccharinate salt.
28. The method of embodiment 27, wherein the saccharin is dissolved in the solvent, before it is added to the 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile in the solvent.
29. The method according to embodiment 27 or 28, wherein the solvent is an alcohol.
30. The method according to embodiment 29, wherein the alcohol is a C₁-C₄ alcohol.
31. The method according to any one of embodiments 28-30, wherein the solvent comprises at least one of ethanol or methanol.
32. The method according to any one of embodiments 27-31, wherein the saccharin is dissolved in the solvent, and the resulting solution is added portionwise to the 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile in the solvent.
33. The method according to any one of embodiments 27-32, wherein the solvent is ethanol and 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9 -dihydrofuro[3,4-f]quinazolin-6-yl)thieno[3,2-c]pyridine-3-carbonitrile in ethanol is heated to about 47±5° C., before the saccharin in ethanol is added.
34. The method according to any one of embodiments 27-33, wherein the saccharin is used in molar excess relative to the 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile.
35. A compound of Formula I:

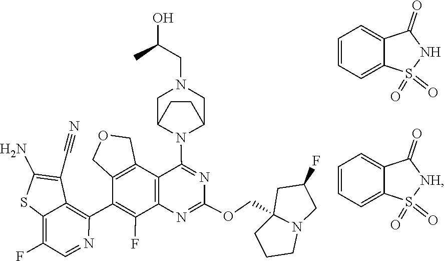

wherein solvent may be optionally present, obtainable by treating 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile with saccharin.
36. A solid oral pharmaceutical dosage form comprising: a) a core composition comprising a compound disclosed herein (e.g., Formula I, Formula II); b) a capsule that contains the core composition and wherein the capsule has a body and a cap; c) a polymeric seal covering the transition between the capsule cap and body; and d) an enteric coating that coats the polymeric seal and capsule.
37. The dosage form of embodiment 36, wherein the compound is a saccharinate salt of

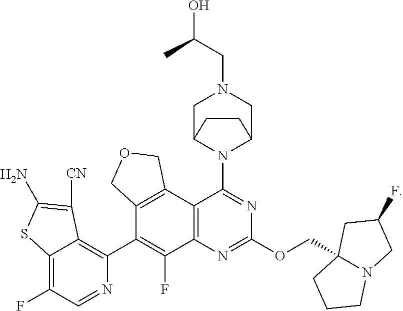 [SMILES: C[C@@H](O)CN1CC2CCC(C1)N2c1nc(OC[C@@]23CCCN2C[C@H](F)C3)nc2c(F)c(-c3ncc(F)c4sc(N)c(C#N)c34)c3c(c12)COC3]

38. The dosage form of embodiment 36, wherein the compound is

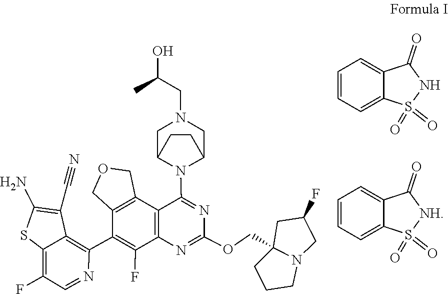

39. The dosage form of embodiment 36, wherein the dosage form releases none of the compound in a fluid having pH<4.5 and releases the majority of the compound in a fluid having pH>6.0.
40. The dosage form of embodiment 36 or 39, wherein the dosage form releases: none of the compounds disclosed herein, for up to 2 hours in fluid having pH between 4.5 and 6.0; and the majority of the compound in fluid having pH>6.0.
41. The dosage form either of embodiment 36 or 39, wherein the dosage form releases: none of the compounds disclosed herein, for up to 2 hours in fluid having pH between 4.5 and 6.0; and the majority of the compound in fluid having pH>6.8.
42. The dosage form of any one of embodiments 36-41, wherein the polymeric seal comprises the same composition as the enteric coating.
43. The dosage form of any one of embodiments 36-42, wherein the polymeric seal comprises HPMC.
44. The dosage form of any of embodiments 36-43, wherein the polymeric seal is a band.
45. The dosage form of any of embodiments 36-44, wherein the enteric coating comprises a copolymer comprising at least one polymer selected from the group consisting of methyl acrylate, methyl methacrylate, methacrylic acid and ethyl acrylate.
46. The dosage form of any of embodiments 36-45, wherein the enteric coating comprises a copolymer comprising at least one polymer selected from polymethacrylic acid and ethyl acrylate.
47. The dosage form of embodiment 45 wherein the methacrylic acid and ethyl acrylate are present in a ratio of about 1:1.
48. The dosage form of embodiment 46, wherein the enteric coating comprises polymethacrylic acid and ethyl acrylate in a concentration between 10 and 70%.
49. The dosage form of embodiment 46, wherein the enteric coating comprises polymethacrylic acid and ethyl acrylate in a concentration of about 65%.
50. The dosage form of any of embodiments 36-38, wherein the coating further comprises polymethyl acrylate, methyl methacrylate, and methacrylic acid.
51. The dosage form of embodiment 45, wherein the methyl acrylate, methyl methacrylate and methacrylic acid are present in a ratio of about 7:3:1.
52. The dosage form of embodiment 50, wherein the enteric coating comprises polymethyl acrylate, methyl methacrylate, and methacrylic acid in a concentration between 40-60%.
53. The dosage form of embodiment 50, wherein the enteric coating comprises polymethyl acrylate, methyl methacrylate, and methacrylic acid in a concentration of about 52%.
54. The dosage form of any one of embodiments 36-53, wherein the enteric coating comprises triethyl citrate (TEC).
55. The dosage form of embodiment 54, wherein the enteric coating comprises TEC in a concentration of about 3%.
56. The dosage form of embodiment 54, wherein the enteric coating comprises TEC in a concentration of about 2%.
57. The dosage form of any one of embodiments 36-55, wherein the coating level is between 5-20 mg/cm².
58. The dosage form of any one of embodiments 36-57, wherein the core composition further comprises a permeation enhancer.
59. The dosage form of embodiment 58, wherein the permeation enhancer is selected from the group consisting of sodium N-[8-(2-hydroxybenzoyl) amino] caprylate (SNAC), sodium caprate (CIO), sodium caprylate (C8), and 8-(N-2-hydroxy-5-chlorobenzoyl)-amino-caprylic acid (5-CNAC).
60. The dosage form of embodiment 58 or 59, wherein the permeation enhancer is CIO.
61. The dosage form of embodiment 60, wherein the core composition comprises between 1 mg and 100 mg of CIO.
62. The dosage form of any one of embodiments 36-61, wherein the core composition further comprises microcrystalline cellulose (MCC).
63. The dosage form of embodiment 62, wherein the core composition comprises about 66 mg MCC.
64. A solid oral pharmaceutical dosage form comprising: a) a core composition comprising a compound disclosed herein (e.g., Formula I, Formula II) and CIO; b) a capsule that contains the core composition and wherein the capsule has a body and a cap; c) a polymeric seal covering the transition between the capsule cap and body; and d) an enteric coating that coats the polymeric seal and capsule, wherein the coating comprises one or more co-polymers selected from the group consisting of: i) polymethacrylic acid, ethyl acrylate], wherein the methacrylic acid and ethyl acrylate are present in a ratio of about 1:1.; ii) and poly[methyl acrylate, methyl methacrylate, methacrylic acid], wherein the methyl acrylate, methyl methacrylate and methacrylic acid are present in a ratio of about 7:3:1.
65. The dosage form of embodiment 64, wherein: the core composition comprises: (i) a compound disclosed herein; and (ii) about 280 mg CIO; the polymeric seal comprises: (i) about 17% HPMC; (ii) about 58% ethanol; and (iii) about 25% water; and the enteric coating comprises: (i) about 12.9% poly[methacrylic acid, ethyl acrylate], wherein the methacrylic acid and ethyl acrylate are present in a ratio of about 1:1.; (ii) about 51.6% polymethyl acrylate, methyl methacrylate, methacrylic acid], wherein the methyl acrylate, methyl methacrylate and methacrylic acid are present in a ratio of about 7:3:1; (iii) about 2.9% TEC; and (iv) about 32.6% water; and wherein the coating level is about 10 mg/cm².
66. The dosage form of embodiment 64, wherein: the core composition comprises: (i) a compound disclosed herein; (ii) about 280 mg CIO; and (iii) about 66 mg MCC; the polymeric seal comprises: (i) about 17% HPMC; (ii) about 58% ethanol; and (iii) about 25% water; and the enteric coating comprises: (i) about 12.9% poly[methacrylic acid, ethyl acrylate], wherein the methacrylic acid and ethyl acrylate are present in a ratio of about 1:1; (ii) about 51.6% polymethyl acrylate, methyl methacrylate, methacrylic acid], wherein the methyl acrylate, methyl methacrylate and methacrylic acid are present in a ratio of about 7:3:1; (iii) about 2.9% TEC; and (iv) about 32.6% water; and wherein the coating level is about 10 mg/cm².
67. The dosage form of embodiment 64, wherein: the core composition comprises: (i) a compound disclosed herein; (ii) about 280 mg CIO; and (iii) about 66 mg MCC; the polymeric seal comprises: (i) about 17% HPMC; (ii) about 58% ethanol; and (iii) about 25% water; and the enteric coating comprises: (i) about 64.5% poly[methacrylic acid, ethyl acrylate], wherein the methacrylic acid and ethyl acrylate are present in a ratio of about 1:1.; (ii) about 2.9% TEC; and (iii) about 32.6% water; and wherein the coating level is about 7 mg/cm².
68. The dosage form of any of embodiments 36-67, wherein the bioavailability of the compound is not negatively impacted if the dosage form is orally administered with food.
69. A process for preparing the dosage form of any one of embodiments 36-68, comprising: a) blending all components comprised in the core composition in a suitable blender; b) weighing an amount of the blend formed in step (a) to be the core composition; c) compressing the blend weighed in step (b) using a capsule slug mold; d) placing the slug formed in step (c) into the capsule; e) covering the transition between the capsule cap and body with the polymeric seal; and f) coating the capsule with the enteric coating.
70. An enteric pellet comprising a) a core consisting of a compound disclosed herein (e.g., Formula I, Formula II) and one or more pharmaceutically acceptable excipients; b) an optional separating layer comprising a non-reducing sugar and one or more pharmaceutically acceptable excipients; c) an enteric layer comprising hydroxypropylmethylcellulose acetate succinate (HPMCAS) and one or more pharmaceutically acceptable excipients; d) an optional finishing layer.
71. A pellet of embodiment 70, wherein the HPMCAS is partially neutralized with ammonium ions to the degree that from about 0% to about 25% of the succinic acid groups are neutralized.
72. A pellet of embodiment 70, wherein the HPMCAS is partially neutralized to the degree that from about 0% to about 15% of the succinic acid groups are neutralized.
73. A pellet of embodiment 70, wherein the separating layer is present.
74. A pellet of embodiment 70, wherein the average particle size of the compound is about 50 μm or less.
75. A pellet of embodiment 70, wherein the core comprises an inert core on which the compound is deposited as a layer comprising in addition a pharmaceutically acceptable excipient.
76. A pellet of embodiment 70, wherein the separating layer is present.
77. A pellet of embodiment 70, wherein the HPMCAS is partially neutralized with ammonium ions to the degree that from about 0% to about 25% of the succinic acid groups are neutralized.
78. A pellet of embodiment 70, wherein the separating layer comprises a pharmaceutically acceptable sugar.
79. A pellet of embodiment 78, wherein the sugar is sucrose.
80. A process for preparing an enteric pellet comprising a) providing a core consisting of a compound as disclosed herein (e.g., Formula I, Formula II) and one or more pharmaceutically acceptable excipients; b) optionally, applying to the core a separating layer comprising a non-reducing sugar and one or more pharmaceutically acceptable excipients; c) applying an enteric layer comprising HPMCAS and one or more pharmaceutically acceptable excipients, wherein the HPMCAS is supplied as an aqueous solution or suspension and the application takes place in an apparatus of the fluid bed type; d) optionally, applying a finishing layer.
81. The process of embodiment 80, wherein the compound is Formula I.
82. The process of embodiment 80, wherein the HPMCAS is fully or partially neutralized with ammonium ions.
83. The process of embodiment 82, wherein the HPMCAS is neutralized to the degree that from about 25% to about 100% of the succinic acid groups are neutralized.
84. The process of embodiment 80, wherein the separating layer is applied.
85. The process of embodiment 84, wherein the separating layer comprises a pharmaceutically acceptable sugar.
86. The process of embodiment 85, wherein the sugar is sucrose.
87. The process of embodiment 80, wherein the core is prepared by applying fluoxetine and one or more pharmaceutically acceptable excipients to an inert core.
88. The process of embodiment 80, wherein the separating layer is applied and comprises a pharmaceutically acceptable sugar.
89. A gelatin capsule containing the pellets of embodiments 70-79.
90. A solid pharmaceutical composition, comprising: (1) a compound disclosed herein (e.g. Formula I, Formula II) constituting about 30-67% percent of the composition; (2) microcrystalline cellulose constituting about 20-55% of the composition; (3) mannitol constituting about 0-10% of the composition; (4) crospovidone constituting about 2% to about 5% of the composition; (5) colloidal silicon dioxide constituting up to about 0.5-2% of the composition; (6) magnesium stearate constituting about 1-3% of the composition, and (7) an enteric coating, wherein all percentages are percentages by weight and wherein the total weight is 100%.
91. The solid pharmaceutical composition of embodiment 90, wherein the compound is Formula I.
92. The solid pharmaceutical composition of embodiment 90, wherein the solid pharmaceutical composition is in the form of a tablet.
93. The solid pharmaceutical composition of embodiment 90, wherein the solid pharmaceutical composition further comprises a film coat.
94. The solid pharmaceutical composition of embodiment 90, wherein (1) the compound disclosed herein (e.g., Formula I, Formula II) constitutes about 33.3% percent of the composition; (2) the microcrystalline cellulose constitutes about 51.2% of the composition; (3) the mannitol constitutes about 10% of the composition; (4) the crospovidone constitutes about 3% of the composition; (5) the colloidal silicon dioxide constitutes about 1% of the composition; (6) the magnesium stearate constitutes about 1.5% of the composition, and (7) an enteric coating, wherein all percentages are percentages by weight and wherein the total weight is 100%.
95. The solid pharmaceutical composition of embodiment 94, wherein the solid pharmaceutical composition is in the form of a tablet.
96. The solid pharmaceutical composition of embodiment 95, wherein the solid pharmaceutical composition further comprises a film coat.
97. An oral solid pharmaceutical composition in the form of a tablet comprising: (1) a compound disclosed herein (e.g., Formula I, Formula II) constituting about 33.3% percent of the composition; (2) microcrystalline cellulose constituting about 51.2% of the composition; (3) mannitol constituting about 10.0% of the composition; (4) crospovidone constituting about 3.0% of the composition; (5) colloidal silicon dioxide constituting about 1.0% of the composition; (6) magnesium stearate constituting about 1.5% of the composition, and (7) an enteric coating, wherein all percentages are percentages by weight and wherein the total weight is 100.0%, and wherein the solid pharmaceutical composition further comprises a film coat.
98. The solid pharmaceutical composition of embodiment 97, wherein the compound is Formula I.
99. A method of treating cancer in a patient in need thereof, the method comprising administering the compound, crystalline form, pharmaceutical composition, dosage form, pellet or solid pharmaceutical composition of any of the preceding embodiments to a patient.
100. The method of embodiment 99, wherein the cancer is a G12D mediated cancer.
101. The method of embodiment 99 or 100, wherein the cancer is selected from the group consisting of lung cancer, pancreatic cancer, cervical cancer, esophageal cancer, endometrial cancer, ovarian cancer, cholangiocarcinoma, and colorectal cancer.
102. The method of embodiment 101, wherein the cancer is selected from the group consisting of pancreatic cancer, colorectal cancer, and non-small cell lung cancer.
103. The method of embodiment 102, wherein the cancer is pancreatic cancer.
104. The method of embodiment 102, wherein the cancer is colorectal cancer.
105. The method of embodiment 102, wherein the cancer is non-small cell lung cancer.
106. The method of any one of embodiments 99-102, wherein the dosage form is administered once daily, twice daily, alternate days, every third day, every fourth day, every fifth day, every sixth day or once weekly.
107. The method of any one of embodiments 99-106, wherein the dosage form is administered once daily.
108. The method of any one of embodiments 99-107, wherein the dosage form may be administered with or without food.
109. A compound, crystalline form, pharmaceutical composition, dosage form, pellet or solid pharmaceutical composition according to any of the prior embodiments, for use in therapy.
110. A compound, crystalline form, pharmaceutical composition, dosage form, pellet or solid pharmaceutical composition according to any of the prior embodiments, for use in the treatment of cancer.
111. The use according to embodiment 110, wherein the cancer is a KRAS G12D associated cancer.
112. The use according to embodiment 111, wherein the cancer comprises lung cancer, pancreatic cancer, cervical cancer, esophageal cancer, endometrial cancer, ovarian cancer, cholangiocarcinoma, or colorectal cancer.
113. The use according to embodiment 111 or 112, wherein the cancer is pancreatic cancer.
114. The use according to embodiment 111 or 112, wherein the cancer is colorectal cancer.
115. The use according to embodiment 111 or 112, wherein the cancer is non-small cell lung cancer.
116. The dosage form for use according to embodiment 110, wherein the dosage form is administered once daily, twice daily, alternate days, every third day, every fourth day, every fifth day, every sixth day or once weekly.
117. The dosage form for use according to embodiment 116, wherein the dosage form is administered once daily.
118. The dosage form for use according to embodiment 116 or 117, wherein the dosage form may be administered with or without food.
119. A method of preparing a solid pharmaceutical composition comprising a compound disclosed herein, the method comprising (a) pre-blending, wherein a diluent, a glidant, and a compound disclosed herein are blended together; (b) de-lumping the components of step (a) using a screening mill; (c) blending the components of step (b) with a disintegrant, and a glidant; (d) lubricating by adding a lubricant to the components of step (c); (e) performing a dry granulation of the components of step (d) using a roller compactor, producing a granulated blend; (f) blending a diluent, a disintegrant and a glidant with the granulated blend of step (e); (g) lubricating by adding a lubricant to the components of step (f) to produce a lubricated blend; (h) performing compression by charging the lubricated blend of step (g) to a rotary tablet press and compressing the lubricated blend into tablet cores; and (i) performing enteric film coating by charging the core tablets of step (h) into a pan coater and adding an enteric film coating agent.
120. The method of embodiment 119, wherein the compound is Formula I.
121. The methods of treatment and uses in therapy, as described above, wherein nausea in the patient receiving the treatment or therapy with an enterically coated dosage form comprising the compound of Formula I is less than the nausea associated with administering a non-enterically coated dosage form of the same amount of the compound of Formula I.
122. The methods of treatment and uses in therapy, as described above, wherein vomiting in the patient receiving the treatment or therapy with an enterically coated dosage form comprising the compound of Formula I is less than the vomiting associated with administering a non-enterically coated dosage form of the same amount of the compound of Formula I.
123. A compound that is

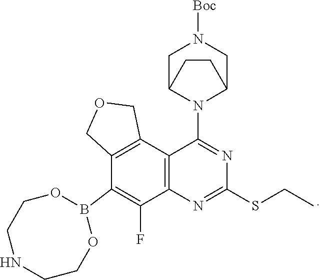 [SMILES: CCSc1nc(N2C3CCC2CN(C(=O)OC(C)(C)C)C3)c2c3c(c(B4OCCNCCO4)c(F)c2n1)COC3]

124. A compound that is

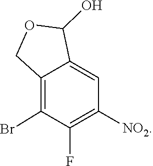 [SMILES: O=[N+]([O-])c1cc2c(c(Br)c1F)COC2O]

125. A compound of Formula I:

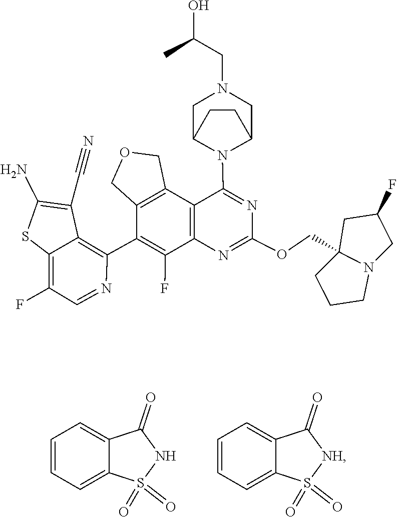 [SMILES: C[C@@H](O)CN1CC2CCC(C1)N2c1nc(OC[C@@]23CCCN2C[C@H](F)C3)nc2c(F)c(-c3ncc(F)c4sc(N)c(C#N)c34)c3c(c12)COC3 ; O=C1NS(=O)(=O)c2ccccc21 ; O=C1NS(=O)(=O)c2ccccc21]

wherein solvent may be optionally present, obtainable by treating 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile with saccharin.
126. The compound of embodiment 125, wherein the saccharin is used in excess.
127. A method of treating gastric cancer comprising administering a compound of Formula I to a patient in need of such treatment.
128. The method according to embodiment 127, wherein the gastric cancer is KRAS G12D gastric cancer.
129. A method of treating a KRAS G12D associated cancer comprising administering a compound of Formula I to a patient in need of such treatment, wherein the KRAS G12D associated cancer comprises colorectal cancer, pancreatic cancer or non-small cell lung cancer.
130. A method of treating a KRAS G12D associated cancer comprising administering a compound of Formula I to a patient in need of such treatment, wherein the KRAS G12D associated cancer comprises colorectal cancer, pancreatic cancer or non-small cell lung cancer.
131. A method of treating a solid tumor that contains one or more cancer cells that express a KRAS G12D mutant protein or contain a KRAS G12D mutation, wherein the method comprises administering a compound of Formula I to a patient in need of such treatment.

### Abbreviations:

“ACN” refers to acetonitrile; “Alloc” refers to the allyloxycarbonyl group; “APC” refers to allylpalladium (II) chloride dimer; “aq.” refers to aqueous; “atm” refers to atmosphere or atmospheres; “B₂pin₂” refers to Bis(pinacolato)diboron: “Boc” refers to tert-butoxycarbonyl; “Cbz” refers to the benzyloxycarbonyl group; “CV” refers to column volumes; “DBDMH” refers to 1,3-dibromo-5,5-dimethylimidazolidine-2,4-dione; “DCM” refers to dichloromethane; “DCE” refers to 1,2-dicloroethane; “DEA” refers to diethanolamine; “DIBAL-H” refers to diisobutylaluminum hydride; “DIEA” and “DIPEA” refer to N,N-diisopropyl ethylamine; “DMA” or “DMAC” refers to dimethylacetamide; “DMAP” refers to 4-dimethylaminopyridine; “DMF” refers to N,N-dimethylformamide; “DMSO” refers to dimethylsulfoxide; “DTT” refers to dithiothreitol; “ERK” refers to extracellular signal-regulated kinases; “EtI” refers to ethyl iodide; “EtOAc” refers to ethyl acetate; “EtOH” refers to ethanol; “F” refers to bioavailability; “Fₐ” refers to fraction absorbed; “FaSIF” refers to fasted simulated intestinal fluid; “FeSIF” refers to fed simulated intestinal fluid; “F_{g}” refers to the fraction that survives metabolism in the gut; “Fmoc” refers to the fluorenylmethyloxycarbonyl group; “GDP” refers to guanosine diphosphate; “GTP” refers to guanosine triphosphate; “h” refers to hour or hours; “Hex” or “hex” refers to hexane or hexanes; “HPLC” refers to high-performance liquid chromatography; “IPA” refers to isopropyl alcohol; “KOAc” refers to potassium acetate; “LiHMDS” refers to lithium bis(trimethylsilyl)amide; “MAPK” refers to mitogen-activated protein kinases; “mCPBA” refers to 3-chloro-peroxybenzoic acid; “Me” refers to a methyl group; “MeOH” refers to methanol; “min” refers to minute or minutes; “MTBE” refers to methyl tert-butyl ether; “NaBH(OAc)₃ refers to sodium triacetoxyborohydride; “NaOMe” refers to sodium methoxide; “NBS” refers to N-bromosuccinimide; “NCS” refers to N-chlorosuccinimide; “NMP” refers to 1-methylpyrrolidin-2-one; “Pd-117” refers to dichloro[bis(2-(diphenylphosphino)phenyl)ether]palladium(II), CAS 205319-06-8; “Pd-170” refers to Chloro(crotyl)(2-dicyclohexylphosphino-2′,4′,6′-triisopropyl-1,1′-biphenyl) palladium(II);(1,2,3-η)-2-Buten-1-yl]chloro[dicyclohexyl[2′,4′,6′-tris(1-methylethyl)[1,1′-biphenyl]-2-yl]phosphine]palladium, CAS 1798782-02-1; “Pd(OAc)₂ refers to palladium (II) acetate; “RT” refers to room temperature; “sat.” refers to saturated; “SCX” refers to strong cation exchange; “SDD” refers to spray dried dispersion; “STAB” refers to Sodium triacetoxyborohydride; “Bu” refers to the tert-butyl group; “TEA” refers to triethylamine; “TFA” refers to trifluoracetic acid; “THF” refers to tetrahydrofuran; “XantPhos” refers to 4,5-bis(diphenylphosphino)-9,9-dimethylxanthene; “XPhos” refers to 2-(dicyclohexylphosphino)-2′,4′,6′-tri-isopropyl-1,1′-biphenyl.

The compounds of the present disclosure, or salts thereof, may be prepared by a variety of procedures, some of which are illustrated in the Schemes, Preparations, and Examples below. The specific synthetic steps for each of the routes described may be combined in different ways, or in conjunction with steps from different routes, to prepare compounds or salts of the present disclosure.

### EXAMPLES

### Synthetic Methods

The following schemes and examples illustrate various syntheses of the compounds disclosed herein.

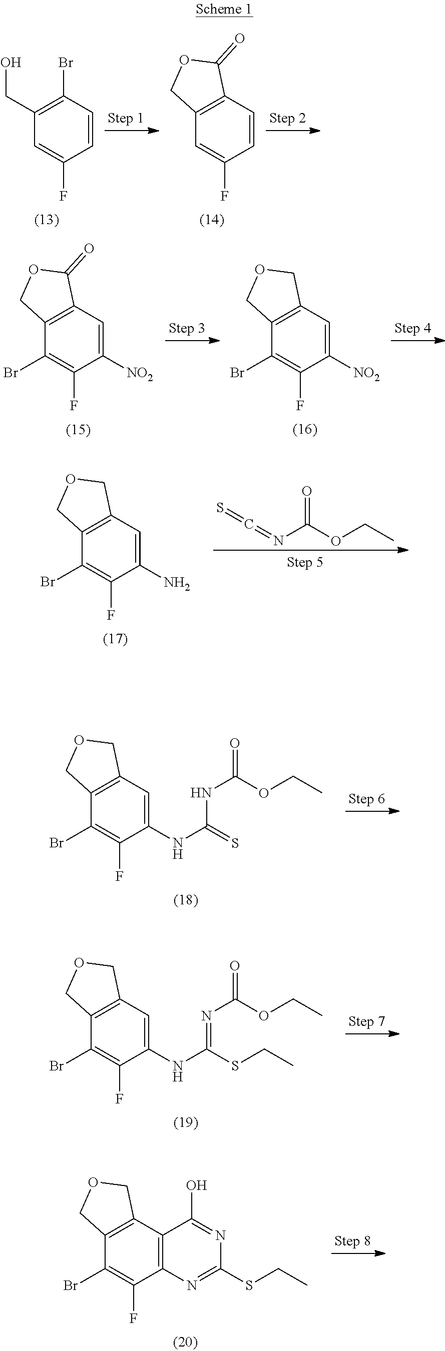

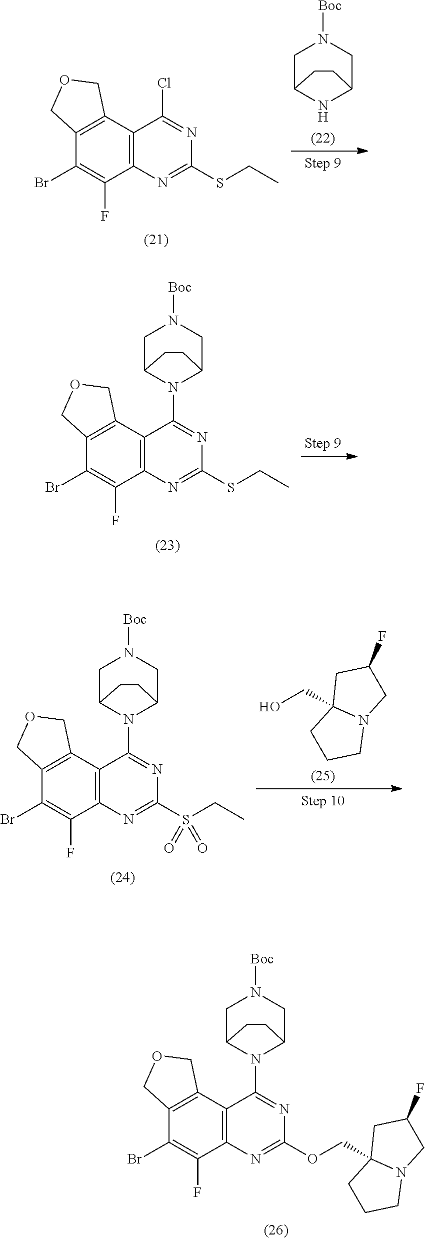 [SMILES: CCSc1nc(Cl)c2c3c(c(Br)c(F)c2n1)COC3 ; CC(C)(C)OC(=O)N1CC2CCC(C1)N2 ; CCSc1nc(N2C3CCC2CN(C(=O)OC(C)(C)C)C3)c2c3c(c(Br)c(F)c2n1)COC3 ; CCS(=O)(=O)c1nc(N2C3CCC2CN(C(=O)OC(C)(C)C)C3)c2c3c(c(Br)c(F)c2n1)COC3 ; OC[C@@]12CCCN1C[C@H](F)C2 ; CC(C)(C)OC(=O)N1CC2CCC(C1)N2c1nc(OC[C@@]23CCCN2C[C@H](F)C3)nc2c(F)c(Br)c3c(c12)COC3]

Scheme 1 depicts the preparation of compound (26) which is used in the preparation of the free base compound (36) (i.e., Formula II). Carbonylation of commercially available compound (13) afford compound (14). A palladium-catalyzed carbonylation may be accomplished using a bidentate ligand such as XantPhos and a palladium source such as Pd(OAc)₂ with a suitable base, for example, triethylamine and a polar aprotic solvent such as acetonitrile. Subsequent nitration of compound (14) followed by bromination can give rise to compound (15). Nitration can be achieved with KNO₃ or HNO₃ in conjunction with a strong acid like H₂SO₄. Bromination may be achieved with a variety of suitable reagents such as, but not limited to, NBS, POBr₃, Br₂, and DBDMH. Selective reduction of compound (15) may yield compound (16). Selective reduction can be achieved using a hydride reagent such as DIBAL-H to give an intermediate lactol compound which is then further reduced by triethylsilane and TFA. Reduction of compound (16) can afford compound (17). Reduction may be achieved with iron or zinc powder and NH₄Cl in a polar solvent such as THF, EtOH or MeOH. Alternatively, the reduction may also be achieved under hydrogenation conditions utilizing a Pt/C catalyst doped with sulfur. Thioacylation of compound (17) can afford compound (18). An appropriate isothiocyanate such as ethoxycarbonyl isothiocyanate can facilitate the transformation. S-Alkylation of compound (18) may yield compound (19). The alkylation may be achieved by using a mild base, such as K₂CO₃, in a polar solvent such as acetone followed by the slow addition of ethyl iodide. Ring closure of compound (19) can afford compound (20). Ring closure may be achieved in a solvent such as anhydrous NMP or diphenyl ether at 175° C. The hydroxy moiety of compounds of (20) may be converted to a leaving group moiety, such as a chloride affording compound (21). Chlorination may be achieved using, for instance, oxalyl chloride, thionyl chloride, (chloromethylene)dimethyliminium chloride or POCl₃ plus adjusting the solvent as necessary. Nucleophilic displacement of chloride compound (21) may afford compound (23). This nucleophilic displace may utilize a substituted piperazine, such as commercially available compound (22), using an appropriate hindered base such as DIPEA in an aprotic solvent such as DMSO. Oxidation of compound (23) may afford compound (24). Oxidation may be accomplished with mCPBA, or other suitable oxidizing agents, along with an appropriate solvent. Nucleophilic displacement of compound (24) may provide compound (25). Nucleophilic displacement maybe be accomplished with a suitable nucleophile, such as a primary or secondary alcohol like commercially available compound (25), using a strong base such as LiHMDS in THF.

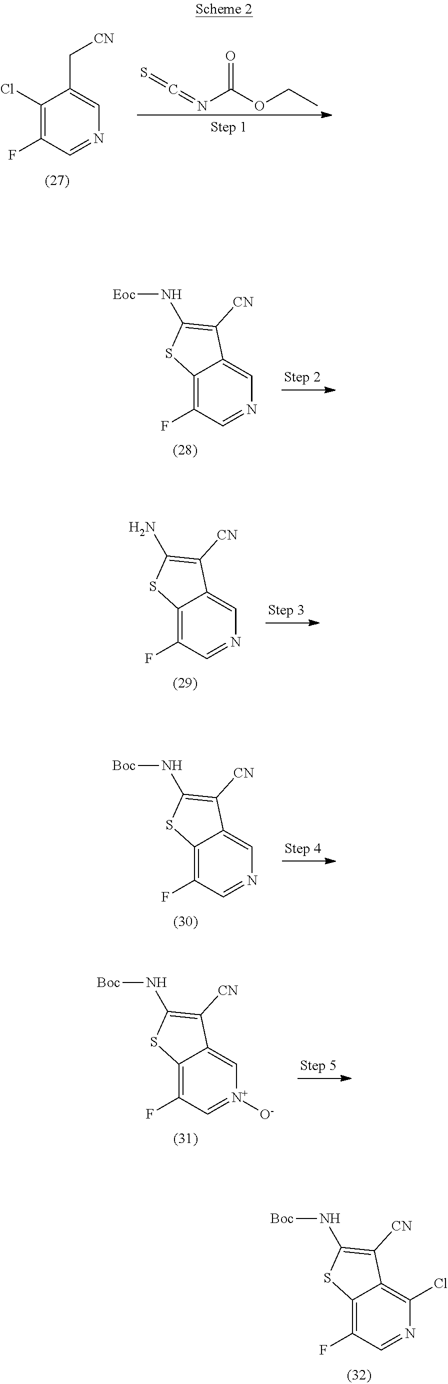

Scheme 2 depicts the preparation of compound (32) which is used in the preparation of the free base compound (36) (i.e., a compound of Formula II). Cyclization of compound (27) with an isothiocyanate gives rise to compound (28). An appropriate isothiocyanate such as ethoxycarbonyl isothiocyanate can facilitate the transformation. Deprotection of compound (28) may afford compound (29). Eoc deprotection can be achieved through basic hydrolysis utilizing hydroxide bases such as NaOH. Protection of compound (29) may provide compound (30). Protection may be accomplished with di-tert-butyl dicarbonate, or Boc group, using a catalytic amount of DMAP under refluxing conditions in acetonitrile. Other suitable carbamate protecting groups such as Alloc, Fmoc and Cbz may also be used in place of the Boc protecting group. Oxidation of compound (30) may provide compound (31). Oxidation may be accomplished with mCPBA, or other suitable oxidizing agents, along with an appropriate solvent. Chlorination of compound (31) may provide compound (32). Chlorination may be achieved using, for instance, oxalyl chloride, thionyl chloride, (chloromethylene)dimethyliminium chloride or POCl₃ plus adjusting the solvent as necessary.

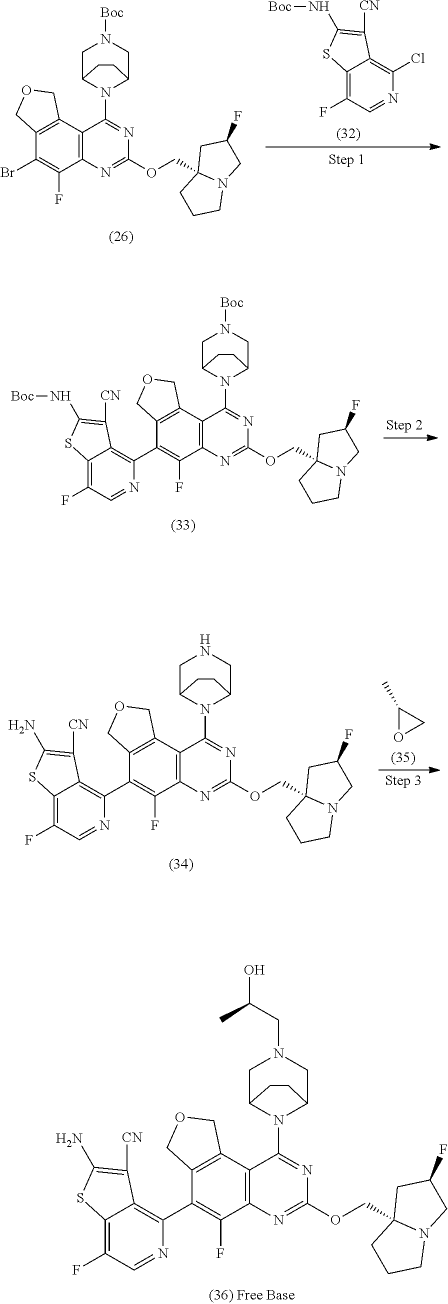

Scheme 3 depicts the preparation of free base (36) (i.e., Formula II) from compounds (26) and (32). Suzuki coupling (26) and compound (32) provide compound (33). The Suzuki coupling may be achieved by reacting a bromide such as compound (26) with bis(neopentyl glycolato)diboron in the presence of a mild base such as KOAc and a palladium complex, such as dichloro[bis(2-(diphenylphosphino)phenyl)ether] palladium(II), to form a boronate ester on the followed by the coupling to chlorothienopyridine, such as compound (32). Deprotection of compound (33) may yield compound (34). Removal of the protecting group(s) may be achieved by methods appropriate to the protecting group used, such as Boc removal by TFA in DCM. N-alkylation of compound (34) may afford free base compound (36). N-alkylation may be achieved with an alkylating agent such as an alkyl halide or an epoxide such as compound (35). Alternatively reductive amination with an appropriate reducing agent and aldehyde may provide the transformation.

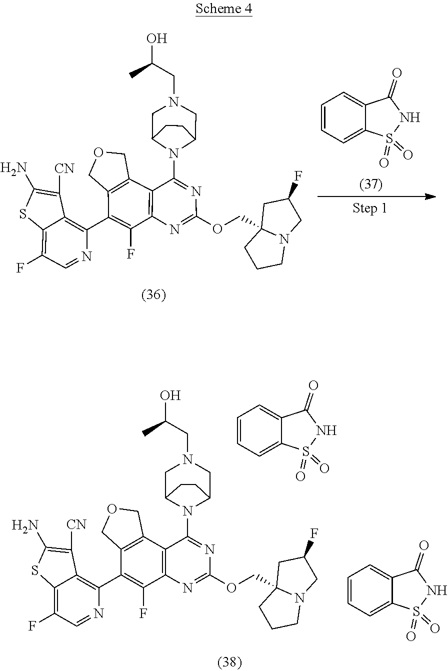

Scheme 4 depicts the preparation of disaccharinate dihydrofuroquinazoline salt compound (38) (i.e., Formula I) from the free base (36) (i.e., Formula II). Combination of free base (36) with compound (37) may afford salt compound (38). Adding saccharin, i.e., compound (37) to a solution of dihydrofuroquinazoline free base compound (36) and subsequent isolation afforded disaccharinate dihydrofuroquinazoline salt compound (38) (i.e., a compound of Formula I). Excess saccharin, relative to the amount of compound (36) used, is preferable.

In an embodiment, the free base (36) (i.e., Formula II) is suspended in a solvent, saccharin is added, and the disaccharinate salt is added. Further, the saccharin may be dissolved in a solvent before it is added to free base (36). In an embodiment, preferred solvents are alcohols. More preferably, the alcohol is a C₁-C₄ alcohol. In an embodiment, the alcohol solvent comprises at least one of ethanol or methanol. In an embodiment, if the saccharin is dissolved in the solvent, the resulting solution is added portionwise to free base (36), which is also in the solvent. In one embodiment, free base (36) is dissolved in ethanol and the resulting solution is heated to about 40 to 78° C., or about 47±5° C., about before the saccharin is added. Alternatively, the solution is heated to not more than about 70° C. As a general rule, the saccharin is used in molar excess, such as 2×, or 2.1×, or 2.2×, or 2.3×, or 2.4×, or 2.5× or more, relative to free base (36).

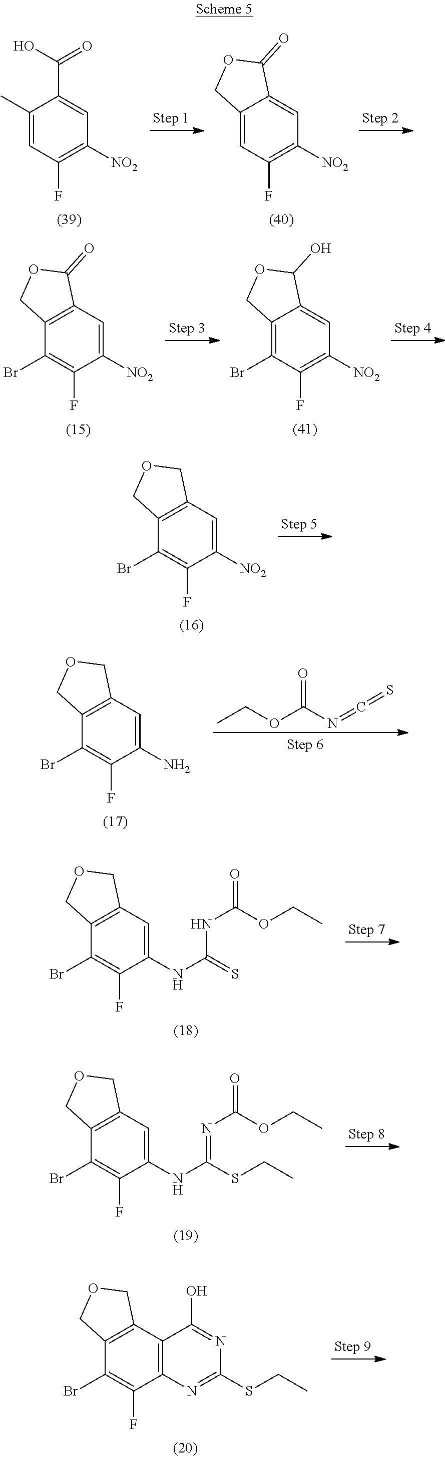

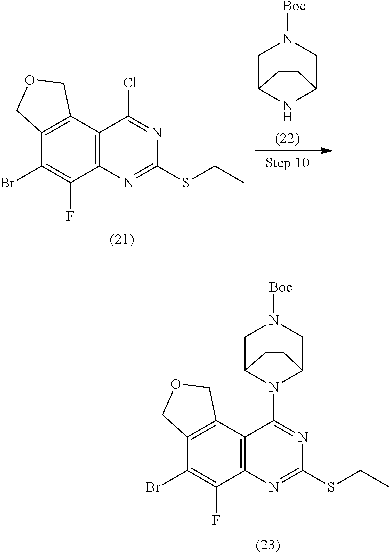 [SMILES: CCSc1nc(Cl)c2c3c(c(Br)c(F)c2n1)COC3 ; CC(C)(C)OC(=O)N1CC2CCC(C1)N2 ; CCSc1nc(N2C3CCC2CN(C(=O)OC(C)(C)C)C3)c2c3c(c(Br)c(F)c2n1)COC3]

Scheme 5 depicts an alternate method of preparing compound (23) which is used in the preparation of the free base compound (36) (i.e., Formula II). In Scheme 8, cyclization of compound (39) may form compound (40). Cyclization may be achieved through benzylic bromination utilizing a brominating agent such as NBS and a radiation source such as Blue LED, followed by lactone ring closure. Bromination of compound (40) may provide compound (15). Bromination may be achieved with a variety of suitable reagents such as, but not limited to, NBS, POBr₃, Br₂, and DBDMH. Reduction of compound (15) may afford compound (41). Selective reduction can be achieved using a hydride reagent such as DIBAL-H. Subsequent reduction of compound (41) can yield compound (16). Treatment with triethylsilane and TFA may facilitate the reduction. Hydrogenation of compound (16) may provide compound (17). Aromatic nitro reduction may be achieved utilizing a catalyst such as Rainy nickel, palladium-on-carbon, or platinum oxide. Thioacylation of compound (17) can afford compound (18). An appropriate isothiocyanate such as ethoxycarbonyl isothiocyanate can facilitate the transformation. S-Alkylation of compound (18) may yield compound (19). The alkylation may be achieved by using a mild base, such as K₂CO₃, in a polar solvent such as acetone followed by the slow addition of ethyl iodide. Ring closure of compound (19) can afford compound (20). Ring closure may be achieved in a solvent such as anhydrous NMP or diphenyl ether at 175° C. Alternatively Eaton's Reagent may be used to facilitate the transformation. The hydroxy moiety of compounds of (20) may be converted to a leaving group moiety, such as a chloride affording compound (21). Chlorination may be achieved using, for instance, oxalyl chloride, thionyl chloride, (chloromethylene)dimethyliminium chloride or POCl₃ plus adjusting the solvent as necessary. Nucleophilic displacement of chloride compound (21) may afford compound (23). This nucleophilic displace may utilize a substituted piperazine, such as commercially available compound (22), using an appropriate hindered base such as DIPEA in an aprotic solvent such as DMSO.

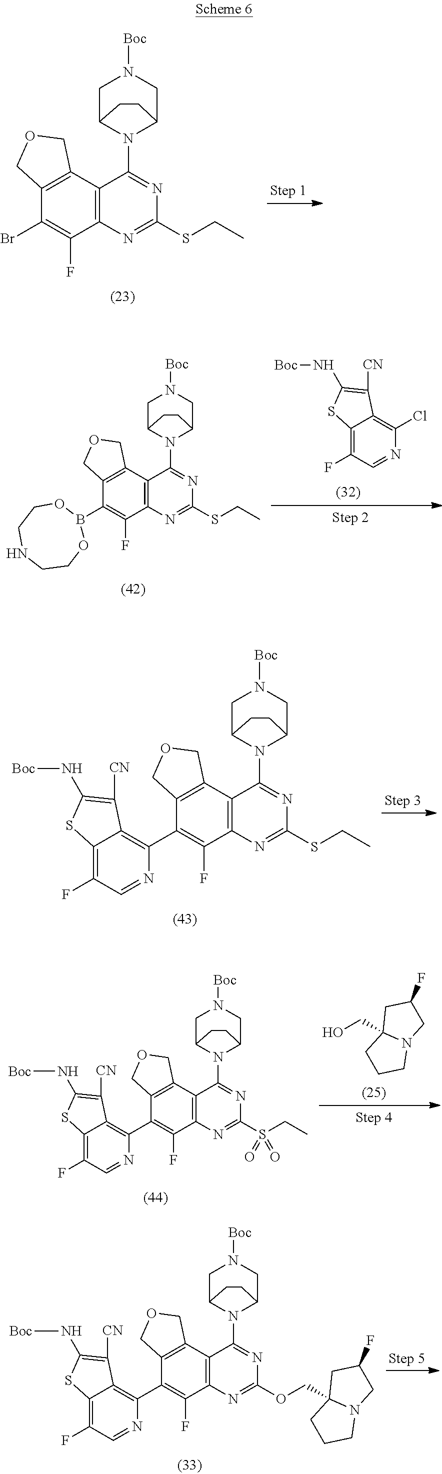

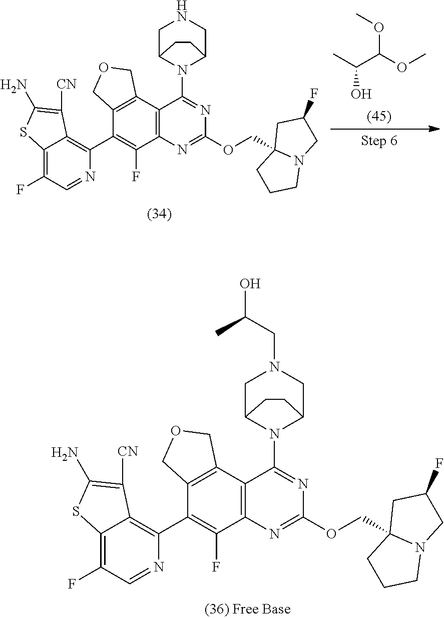

Scheme 6 depicts the preparation of free base compound (36) (i.e., Formula II) from compound (23) and compound (32). Borylation of compound (23) may provide compound (42). Borylation may be achieved by reacting a bromide such as compound (23) with a diboron complex such as B₂pin₂ in the presence of a mild base such as NaHCO₃ and a palladium complex, such as Pd-117, subsequent boronate ester swap with an appropriate diol such as DEA may afford a boronate ester such as compound (42). Suzuki coupling of compound (42) and compound (32) may provide compound (43). The Suzuki coupling may be achieved by reacting a boronate ester such as compound (42) in the presence of a mild base such as KOAc and a palladium complex, such Pd-170 with a chlorothienopyridine, such as compound (32). Oxidation of compound (43) may afford compound (44). Oxidation may be accomplished with mCPBA, or other suitable oxidizing agents, along with an appropriate solvent. Nucleophilic displacement of compound (44) may provide compound (33). Nucleophilic displacement maybe be accomplished with a suitable nucleophile, such as a primary or secondary alcohol like commercially available compound (25), using a strong base such as LiO^{t}Bu in a polar solvent such as DMA. Deprotection of compound (33) may yield compound (34). Removal of the protecting group(s) may be achieved by methods appropriate to the protecting group used, such as Boc removal by TFA in DCM. N-alkylation of compound (34) may afford free base compound (36). N-alkylation may be achieved through reductive amination with an appropriate reducing agent such as STAB and an aldehyde or an aldehyde derived in situ from an acetal such as (45). Alternatively N-alkylation can be achieved with an alkyl halide or epoxide.

### Preparation 1 5-Fluoroisobenzofuran-1(3H)-one

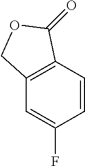 [SMILES: O=C1OCc2cc(F)ccc21]

To a stirred mixture of (2-bromo-5-fluorophenyl)methanol (500 g, 2.44 mol) and TEA (474.6 mL, 3.41 mol, 1.4 eq.) in ACN (2500 mL) was added Pd(OAc)₂ (10.95 g, 48.77 mmol, 0.02 eq.) and XantPhos (42.33 g, 73.16 mmol, 0.03 equiv.) at RT, then stirred for 3 days at 120° C. under 10 atm of carbon monoxide. The reaction was cooled to RT and concentrated. The residue was diluted with H₂O (1,000 mL), then extracted with EtOAc (2×2000 mL). The combined organic layers were washed with brine (2×1,000 mL), dried over anhydrous Na₂SO₄, filtered and concentrated. The residue was triturated with 10:1 hexanes/EtOAc (1,100 mL) and then filtered. The filter cake was dried at 50° C. for ~18 h to obtain the title compound as a yellow solid (300 g, 81%). MS (ES) m/z=153 (M+1).

### Preparation 2 4-Bromo-5-fluoro-6-nitroisobenzofuran-1(3H)-one

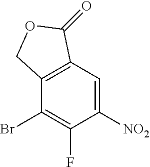 [SMILES: O=C1OCc2c1cc([N+](=O)[O-])c(F)c2Br]

To a stirred mixture of 5-fluoro-3H-isobenzofuran-1-one (300 g, 1.97 mol) in H₂SO₄ (1,500 mL) was added HNO₃ (273.38 g, 4.348 mol, 2.2 eq.) dropwise at 65° C. The reaction was stirred for 1 h then cooled to RT. 1,3-dibromo-5,5-dimethylimidazolidine-2,4-dione (2,255.43 g, 7.88 mol, 4 eq.) was added in portions over 20 min and was stirred at RT for ~18 h. The mixture was poured onto ice/water (pre-treated with 3 kg Na₂SO₃) and filtered. The filter cake was dissolved in EtOAc (3,000 mL), washed with sat. aq. Na₂CO₃ (2×1,000 mL), brine (2×1,000 mL), dried over anhydrous Na₂SO₄ and concentrated. The residue was triturated with 10:1 hexanes/EtOAc (660 mL) and was filtered and dried at 50° C. for ~18 h to obtain the title compound as a yellow solid (270 g, 49%) which was used in a subsequent step without further purification. ¹H NMR (400 MHz, DMSO-d₆) δ 8.58 (s, 1H), 5.51 (s, 2H).

### Preparation 3 4-Bromo-5-fluoro-6-nitro-1,3-dihydroisobenzofuran

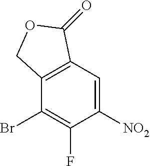 [SMILES: O=C1OCc2c1cc([N+](=O)[O-])c(F)c2Br]

To a stirred mixture of 4-bromo-5-fluoro-6-nitro-3H-isobenzofuran-1-one (270 g, 978 mmol) in DCM (2,500 mL) was added DIBAL-H (1M in THF, 1,467 mL, 1.467 mol, 1.5 eq.) dropwise at −78° C. under N₂. The reaction was stirred for 5 h at −78° C., then was quenched with 5N NaOH (300 mL) at −78° C. The resulting mixture was allowed to warm to RT, then was concentrated. The residue was diluted with EtOAc (2,500 mL), washed with brine (2×1,000 mL) and dried over anhydrous Na₂SO₄ and concentrated. The residue was triturated with 10:1 hexanes/EtOAc (550 mL) and filtered. The solids were dried (190 g, 683.4 mmol) then dissolved in DCM (1,500 mL) and treated dropwise with Et₃SiH (662 mL, 4.10 mol, 6 eq.) at 0° C. The reaction was stirred for 20 min at 0° C. TFA (152 mL, 2.05 mol, 3 eq.) was added dropwise at 0° C. The ice bath was removed, and the reaction was stirred at RT for ~18 h. The reaction was concentrated to an oil, which was diluted with EtOAc (2,000 mL), washed with sat. aq. Na₂CO₃ (2×500 mL) and brine (2×500 mL), dried over anhydrous Na₂SO₄, filtered and concentrated to obtain the title compound (110 g, 42%) which was used in a subsequent step without further purification. ¹H NMR (400 MHz, DMSO-d₆) δ 8.16 (d, J=6.2 Hz, 1H), 5.18-5.15 (m, 2H), 5.11-5.06 (m, 2H).

### Preparation 4 7-Bromo-6-fluoro-1,3-dihydroisobenzofuran-5-amine

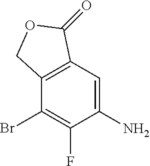 [SMILES: Nc1cc2c(c(Br)c1F)COC2=O]

To a stirred mixture of 4-bromo-5-fluoro-6-nitro-1,3-dihydroisobenzofuran (110 g, 420 mmol) and NH₄Cl (112.3 g, 2.10 mol, 5 eq.) in EtOH (1,000 mL) and H₂O (200 mL) was added Fe (117.22 g, 2.09 mol, 5 eq.) in portions at RT, then stirred for ~18 h at 80° C. The mixture was filtered and concentrated. The mixture was diluted with H₂O (500 mL) and extracted with EtOAc (2×1,000 mL). The combined organic layers were washed with brine (2×500 mL), dried over anhydrous Na₂SO₄, filtered and concentrated. The residue was purified on silica (25% to 50% EtOAc/Hex) to afford the title compound (70 g, 72%) as a yellow solid. MS (ES) m/z=231 (M+1).

### Preparation 5 Ethyl N-[(7-bromo-6-fluoro-1,3-dihydroisobenzofuran-5-yl)carbamothioyl]carbamate

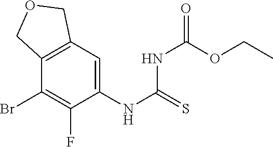 [SMILES: CCOC(=O)NC(=S)Nc1cc2c(c(Br)c1F)COC2]

A solution of 7-bromo-6-fluoro-1,3-dihydroisobenzofuran-5-amine (20.4 g, 87.9 mmol) in DCM (550 mL) was charged with ethoxycarbonyl isothiocyanate (9.7 mL, 82 mmol, 0.93 eq.) slowly via addition funnel and subsequently stirred at RT for ~4 h. The solids were filtered. The filtrate was concentrated, suspended in DCM (100 mL) and hexanes (350 mL) and stirred at RT. The resultant filtered solids and previous filtered solids were dried under vacuum at 50° C. for 2 h. The batches were combined to obtain the title compound (32.6 g, quantitative) as a white solid. MS (ES) m/z=363 (M+1).

### Preparation 6 Ethyl (NZ)—N-[[(7-bromo-6-fluoro-1,3-dihydroisobenzofuran-5-yl)amino]-ethylsulfanyl-methylene]carbamate; or Ethyl (Z)-(((7-bromo-6-fluoro-1,3-dihydroisobenzofuran-5-yl)amino)(ethylthio)methylene)carbamate

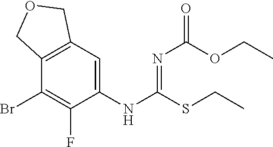 [SMILES: CCOC(=O)/N=C(/Nc1cc2c(c(Br)c1F)COC2)SCC]

A 2 L 3-necked RBF, equipped with an overhead stirrer, dropping funnel and thermocouple was charged with a suspension of ethyl N-[(7-bromo-6-fluoro-1,3-dihydroisobenzofuran-5-yl)carbamothioyl]carbamate (32.6 g, 89.8 mmol) and acetone (450 mL). To this was added solid K₂CO₃ (37.2 g, 269 mmol, 3.00 eq.) in several portions, followed by the dropwise addition of EtI (7.2 mL, 90 mmol, 1.0 eq.) over 20 min. The mixture was stirred at RT for ~18 h. The solids were filtered and the filtrate was concentrated and partitioned between DCM (500 mL) and H₂O (500 mL). The organics were further washed with brine and dried over anhydrous Na₂SO₄, filtered and concentrated. The residue was purified on silica (0 to 30% EtOAc/Hex) to obtain the title compound (30.9 g, 85.6%) as a white solid. MS (ES) m/z=391 (M+1).

### Preparation 7 6-Bromo-3-(ethylthio)-5-fluoro-7,9-dihydrofuro[3,4-f]quinazolin-1-ol

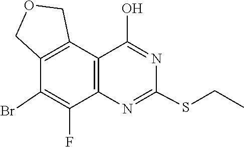 [SMILES: CCSc1nc(O)c2c3c(c(Br)c(F)c2n1)COC3]

A 2 L 4-necked RBF was equipped with an overhead stirrer, dropping funnel, N₂ inlet and thermocouple and was purged with N₂. NMP (anhydrous, 300 mL) was added. The mixture was heated to 175° C. In a second flask, ethyl (NZ)—N-[[(7-bromo-6-fluoro-1,3-dihydroisobenzofuran-5-yl)amino]-ethylsulfanyl-methylene]carbamate (22.63 g, 57.83 mmol) and NMP (anhydrous, 100 mL) were combined and stirred under N₂ until a homogeneous solution was obtained. When the first flask had reached 175° C., the contents of the second flask were poured into the dropping funnel and were added dropwise but rapidly to the hot NMP. After 30 min, the heat was turned off and the reaction cooled to 45° C. H₂O (500 mL) was slowly added and the mixture was stirred at RT for 1 h. The solids were filtered, rinsed with H₂O (300 mL) and dried under vacuum at 50° C. for ~18 h to afford the title compound (15.2 g, 73%) as an off-white solid. MS (ES) m/z=363 (M+1).

### Preparation 8 6-Bromo-1-chloro-3-(ethylthio)-5-fluoro-7,9-dihydrofuro[3,4-f]quinazoline

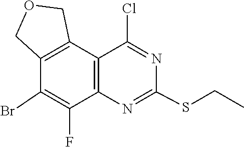 [SMILES: CCSc1nc(Cl)c2c3c(c(Br)c(F)c2n1)COC3]

A 5 L 3-necked RBF, equipped with a dropping funnel, thermocouple and an overhead stirrer was charged with a solution of DMF (50 mL, 646 mmol, 4 eq.) in DCM (1,000 mL) and was placed in an ice/water bath and cooled to ~4° C. Oxalyl chloride (50.0 mL, 576 mmol, 4 eq.) was added dropwise via addition funnel over ~40 min. When the addition was complete, the reaction was stirred at ~4° C. for 15 min. Solid 6-bromo-3-ethylsulfanyl-5-fluoro-7,9-dihydrofuro[3,4-f]quinazolin-1-ol (50.4 g, 140 mmol) was added in several portions to the reaction mixture and the resulting suspension was stirred at ~4° C. for 30 min. The ice bath was removed and the reaction was allowed to warm to RT and stir for 1 h. Then H₂O (1 L) was added and the mixture was stirred for 15 min. The mixture was partitioned and the organic layer was washed with brine (1 L) and dried over anhydrous Na₂SO₄, filtered and concentrated. The residue was purified on silica, eluting with DCM/Hex (60% to 90%) to obtain the title compound (45.1 g, 89%) as a white solid. MS (ES) m/z=363 (M+1).

### Preparation 9 tert-Butyl 8-(6-bromo-3-(ethylthio)-5-fluoro-7,9-dihydrofuro[3,4-f]quinazolin-1-yl)-3,8-diazabicyclo[3.2.1]octane-3-carboxylate

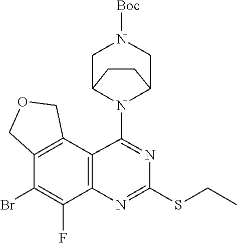 [SMILES: CCSc1nc(N2C3CCC2CN(C(=O)OC(C)(C)C)C3)c2c3c(c(Br)c(F)c2n1)COC3]

A suspension of 6-bromo-1-chloro-3-ethylsulfanyl-5-fluoro-7,9-dihydrofuro[3,4-f]quinazoline (21.0 g, 57.8 mmol) in ACN (580 mL) was charged with tert-butyl 3,8-diazabicyclo[3.2.1]octane-3-carboxylate (15.2 g, 69.5 mmol, 1.20 eq.) and DIPEA (40 mL, 229 mmol, 4 eq.) and was stirred at RT for 90 min. H₂O (1 L) was added slowly via addition funnel and the mixture was stirred at RT for 1 h. The solids were filtered, rinsed with H₂O (500 mL) and dried under vacuum at 50° C. to obtain the title compound (31 g, quantitative) as a white solid, MS (ES) m/z=539 (M+1).

### Preparation 10 tert-Butyl 8-(6-bromo-3-(ethylsulfonyl)-5-fluoro-7,9-dihydrofuro[3,4-f]quinazolin-1-yl)-3,8-diazabicyclo[3.2.1]octane-3-carboxylate

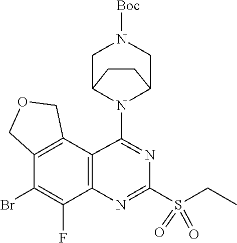 [SMILES: CCS(=O)(=O)c1nc(N2C3CCC2CN(C(=O)OC(C)(C)C)C3)c2c3c(c(Br)c(F)c2n1)COC3]

A solution of tert-butyl 8-(6-bromo-3-ethylsulfanyl-5-fluoro-7,9-dihydrofuro[3,4-f]quinazolin-1-yl)-3,8-diazabicyclo[3.2.1]octane-3-carboxylate (4.3 g, 8.0 mmol) in DCM (40 mL) was charged with mCPBA (6.21 g, 25.2 mmol, 3.2 eq.) and stirred at RT for 90 min. The mixture was diluted with DCM and washed with sat. aq. NaHCO₃, and brine. The organics were dried over anhydrous Na₂SO₄, filtered and concentrated. The residue was purified on silica, eluting with EtOAc/Hex (20-80%) to obtain the title compound (3.0 g, 66%) as a white solid. MS (ES) m/z=571 (M+1).

### Preparation 11 tert-Butyl 8-(6-bromo-5-fluoro-3-(((2R,7aS)-2-fluorotetrahydro-1H-pyrrolizin-7a(5H)-yl)methoxy)-7,9-dihydrofuro[3,4-f]quinazolin-1-yl)-3,8-diazabicyclo[3.2.1]octane-3-carboxylate

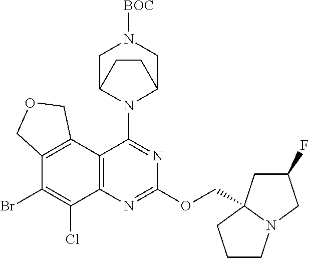 [SMILES: CC(C)(C)OC(=O)N1CC2CCC(C1)N2c1nc(OC[C@@]23CCCN2C[C@H](F)C3)nc2c(Cl)c(Br)c3c(c12)COC3]

To a mixture of [(2R,8S)-2-Fluoro-1,2,3,5,6,7-hexahydropyrrolizin-8-yl]methanol (2.26 g, 13.8 mmol) in THF (110 mL) under N₂ was added dropwise LiHMDS in THF (13.7 mL, 13.7 mmol, 1.0 M) and stirred for 20 min. To this solution was added dropwise via syringe, tert-butyl 8-(6-bromo-3-ethylsulfonyl-5-fluoro-7,9-dihydrofuro[3,4-f]quinazolin-1-yl)-3,8-diazabicyclo[3.2.1]octane-3-carboxylate (6.54 g, 11.4 mmol) in THF (40 mL). After 35 min, the reaction was diluted with EtOAc (250 mL) and washed with brine. The aqueous was extracted with EtOAc (2×200 mL) and the combined organics were dried over Na₂SO₄ and concentrated to a tan solid. The crude material was purified by silica, eluting with 0-10% MeOH/DCM to afford the title compound (7.0 g, 96%) as a light-tan solid. MS (ES) m/z=636 (M+1).

### Preparation 12 Ethyl (3-cyano-7-fluorothieno[3,2-c]pyridin-2-yl)carbamate

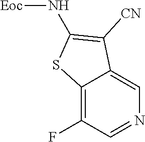

A solution of 2-(4-chloro-5-fluoropyridin-3-yl)acetonitrile (11.8 g, 56.1 mmol) in DMF (112 mL) was cooled to 0° C. Potassium tert-butoxide (7.00 g, 61.1 mmol) was added. After 15 min, ethoxycarbonyl isothiocyanate (7.45 mL, 61.8 mmol) was added dropwise. The reaction mixture was allowed to slowly warm to room temperature overnight. The reaction mixture was poured into a mixture of ice/water (1.5 L), stirred until all ice had melted, and filtered through diatomaceous earth. The solids were dried in a vacuum oven (60° C.) overnight and separated from the diatomaceous earth to give ethyl N-(3-cyano-7-fluoro-thieno[3,2-c]pyridin-2-yl)carbamate (11.9 g, 79%) as a solid. MS (ES) m/z=266 (M+1).

### Preparation 13 2-Amino-7-fluorothieno[3,2-c]pyridine-3-carbonitrile

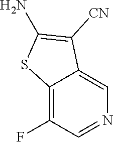 [SMILES: N#Cc1c(N)sc2c(F)cncc12]

A suspension of ethyl (3-cyano-7-fluorothieno[3,2-c]pyridin-2-yl)carbamate (11.9 g, 44.4 mmol) in DMSO (90 mL) was cooled to 0° C. NaOH (5 M in water, 90 mL) was added dropwise over 15 min. The reaction mixture was heated to 105° C. for 1 h, then cooled to room temperature. The reaction mixture was poured into a mixture of ice/water (1.8 L), stirred until all ice had melted, and filtered through diatomaceous earth. The solids were dried in a vacuum oven (50° C.) overnight and separated from the diatomaceous earth to give crude 2-amino-7-fluoro-thieno[3,2-c]pyridine-3-carbonitrile.

### Preparation 14 tert-Butyl (3-cyano-7-fluorothieno[3,2-c]pyridin-2-yl)carbamate

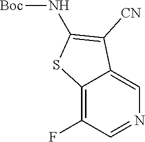 [SMILES: CC(C)(C)OC(=O)Nc1sc2c(F)cncc2c1C#N]

A mixture of crude 2-amino-7-fluorothieno[3,2-c]pyridine-3-carbonitrile (8.6 g, 44.4 mmol), DCM (90 mL), DMF (90 mL) and N,N-diisopropylethylamine (15.5 mL, 88.9 mmol) was cooled to 0° C. 4-dimethylaminopyridine (0.54 g, 4.42 mmol) and di-tert-butyl dicarbonate (14.6 g, 66.7 mmol) were added. The reaction mixture was stirred at room temperature for 2 h. The solvents were removed under reduced pressure and the remaining material was diluted with DCM (400 mL) and 5% aq. citric acid (250 mL). The aqueous phase was washed twice with DCM. The combined organic phases were washed with sat. aq. NaHCO₃, dried over MgSO₄, filtered, and concentrated to give tert-butyl N-(3-cyano-7-fluoro-thieno[3,2-c]pyridin-2-yl)carbamate (7.5 g, 58%) as a brown solid. MS (ES) m/z=294 (M+1).

### Preparation 15 2-((tert-Butoxycarbonyl)amino)-3-cyano-7-fluorothieno[3,2-c]pyridine 5-oxide

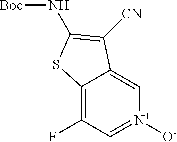 [SMILES: CC(C)(C)OC(=O)Nc1sc2c(F)c[n+]([O-])cc2c1C#N]

mCPBA (9.00 g, 40.2 mmol) was added to a solution of tert-butyl (3-cyano-7-fluorothieno[3,2-c]pyridin-2-yl)carbamate (7.85 g, 26.8 mmol) in DCM (180 mL). The reaction mixture was stirred at room temperature overnight, then cooled to 0° C. for ~15 min. Solids were collected by filtration and dried in a vacuum oven (60° C.). The filtrate was diluted with MeOH and silica gel, concentrated, and the residue was purified on silica, eluting with 0-6% MeOH in DCM. Fractions containing desired material were combined with the solids from the filtration and concentrated to give tert-butyl N-(3-cyano-7-fluoro-5-oxido-thieno[3,2-c]pyridin-5-ium-2-yl)carbamate (7.26 g, 88%) as an off-white solid. MS (ES) m/z=310 (M+1).

### Preparation 16 tert-Butyl (4-chloro-3-cyano-7-fluorothieno[3,2-c]pyridin-2-yl)carbamate

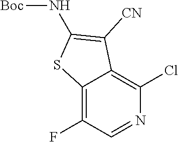 [SMILES: CC(C)(C)OC(=O)Nc1sc2c(F)cnc(Cl)c2c1C#N]

A suspension of 2-((tert-butoxycarbonyl)amino)-3-cyano-7-fluorothieno[3,2-c]pyridine 5-oxide (5.27 g, 17.0 mmol) in 1,2-dichloroethane (34 mL) was cooled to 0° C. A solution of phosphoryl chloride (32 mL, 344 mmol) in 1,2-dichloroethane (34 mL) was added dropwise. The reaction mixture was stirred at room temperature for 30 min, at 45° C. for 90 min, and cooled to room temperature. The reaction mixture was diluted with 1,2-dichloroethane (100 mL) and added to a mixture of sat. aq. NaHCO₃ (500 mL), NaOH (5 M in water, 40 mL), and ice. Solid NaHCO₃ was added to the stirred mixture to maintain pH ~6-7. Once bubbling ceased, the phases were separated. The aqueous phase was extracted 3× with DCM. The combined organic phases were dried over MgSO₄ and filtered. The filtrate was diluted with MeOH and silica gel, concentrated, and the residue was purified on silica, eluting with 50-100% DCM in hexanes. Fractions containing desired material were concentrated to give the title compound (3.87 g, 69%) as a white solid. MS (ES) m/z=328 (M+1).

### Preparation 17 tert-Butyl 8-(6-(2-((tert-butoxycarbonyl)amino)-3-cyano-7-fluorothieno[3,2-c]pyridin-4-yl)-5-fluoro-3-(((2R,7aS)-2-fluorotetrahydro-1H-pyrrolizin-7a(5H)-yl)methoxy)-7,9-dihydrofuro[3,4-f]quinazolin-1-yl)-3,8-diazabicyclo[3.2.1]octane-3-carboxylate

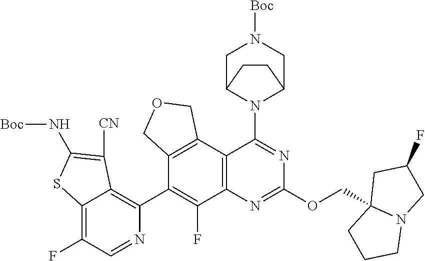 [SMILES: CC(C)(C)OC(=O)Nc1sc2c(F)cnc(-c3c4c(c5c(N6C7CCC6CN(C(=O)OC(C)(C)C)C7)nc(OC[C@@]67CCCN6C[C@H](F)C7)nc5c3F)COC4)c2c1C#N]

A mixture K₃PO₄ in H₂O (0.98 mL, 2.0 mmol, 2 M) and 1,4-dioxane (12.5 mL) was sparged with N₂ for 30 min. tert-Butyl 8-(6-bromo-5-fluoro-3-(((2R,7aS)-2-fluorotetrahydro-1H-pyrrolizin-7a(5H)-yl)methoxy)-7,9-dihydrofuro[3,4-f]quinazolin-1-yl)-3,8-diazabicyclo[3.2.1]octane-3-carboxylate (0.500 g, 0.786 mmol), B₂pin₂ (0.242 g, 0.943 mmol), XPhos (0.038 g, 0.078 mmol), and aq. (0.016 g, 0.040 mmol) was added, and the resulting mixture was heated at 100° C. for ~18 h. The reaction mixture was cooled to RT and was partitioned between DCM and H₂O. The layers were separated, and the aqueous layer was extracted 1× with DCM and extracted 2× with 4:1 CHCl₃:IPA. The organic layers were combined, dried over Na₂SO₄ and concentrated in vacuo. The residue was purified on silica (gradient 0 to 60%, 10% 1N NH₃/MeOH in DCM) to obtain the title compound (0.306 g, 61%) as a orangish-brown solid. MS (ES) m/z=849 (M+1).

### Preparation 18 4-(1-(3,8-Diazabicyclo[3.2.1]octan-8-yl)-5-fluoro-3-(((2R,7aS)-2-fluorotetrahydro-1H-pyrrolizin-7a(5H)-yl)methoxy)-7,9-dihydrofuro[3,4-f]quinazolin-6-yl)-2-amino-7-fluorothieno[3,2-c]pyridine-3-carbonitrile

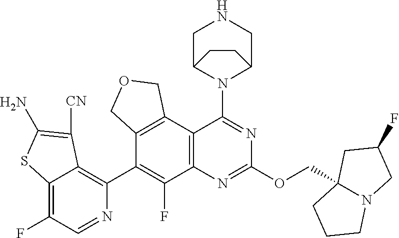 [SMILES: N#Cc1c(N)sc2c(F)cnc(-c3c4c(c5c(N6C7CCC6CNC7)nc(OC[C@@]67CCCN6C[C@H](F)C7)nc5c3F)COC4)c12]

A solution of tert-butyl 8-(6-(2-((tert-butoxycarbonyl)amino)-3-cyano-7-fluorothieno[3,2-c]pyridin-4-yl)-5-fluoro-3-(((2R,7aS)-2-fluorotetrahydro-1H-pyrrolizin-7a(5H)-yl)methoxy)-7,9-dihydrofuro[3,4-f]quinazolin-1-yl)-3,8-diazabicyclo[3.2.1]octane-3-carboxylate (0.301 g, 0.355 mmol) in DCM (4 mL) was charged with TFA (1.5 mL) and stirred at RT for 5 hr. The mixture was concentrated, DCM was added, and the mixture was concentrated again, repeating 1×. The mixture was filtered through an SCX column, eluting with MeOH (4 CV), followed by 1:1 2M ammonia/MeOH: DCM (4 CV) to obtain the crude product, which was purified on silica gel, eluting with a gradient of 20 to 100% [20% (1N NH3 MeOH)/DCM) in DCM] to obtain the title compound (0.059 g, 12%) as a pale yellow solid. MS (ES) m/z=649 (M+1).

### Preparation 19 5-Fluoro-6-nitroisobenzofuran-1(3H)-one

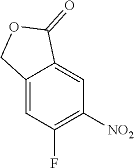 [SMILES: O=C1OCc2cc(F)c([N+](=O)[O-])cc21]

4-Fluoro-2-methyl-5-nitrobenzoic acid was combined with (100 g, 502 mmol) ACN (3000 mL) NBS (69 g, 552 mmol) and Charge Citric acid monohydrate (5.27 g, 25.1 mmol). The reaction mixture was continuously transferred through a flow system. The temperature of the flow system was maintained between 28-40° C. while exposing the reaction mixture to Blue LED (440-460 nm). The reaction mixture exited the flow system into aq. NaHCO₃ and aq. Na₂SO₃ solution. The solution was concentrated to between 2000-2600 mL at below 35° C. Additional aq. NaHCO₃ solution was added. The aqueous layer was extracted with EtOAc (2×700 vol). The combined organic layers were concentrated under vacuum at 40° C. to 100-200 mL. Water (300 mL) was added, and the resultant solid was isolated by filtration. The solid was washed with water (150 mL). The solid was dried at 45-55° C. to afford title compound (40.15 kg, 74%). MS (ES) m/z=196 (M-1).

### Preparation 20 4-Bromo-5-fluoro-6-nitroisobenzofuran-1(3H)-one

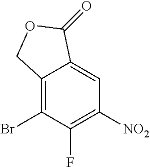 [SMILES: O=C1OCc2c1cc([N+](=O)[O-])c(F)c2Br]

A solution of 5-fluoro-6-nitroisobenzofuran-1(3H)-one (100 g, 507 mmol) in H₂SO₄ (796 mL) at 25-30° C. and a solution of 1,3-Dibromo-5,5-Dimethylhydantoin (DMDBH, 243.68 g, 852 mmol) in H₂SO₄ (2506 mL) at 25-30° C. were continuously fed through a flow reaction system. The reaction mixture was fed through the first flow reactor heated to 80-110° C. The reaction was fed through the second flow reactor cooled to 2-8° C. The reaction mixture was fed to a holding chamber at 2-8° C. The reaction mixture was then fed dropwise to water (3000 mL) at 5-15° C. controlling the exotherm. The mixture was cooled to −5-5° C. over 1-4 h. The resultant solid was collected by filtration and washed with water (500 mL). The solid was then stirred with water (500 mL) and stirred for 2-4 h at 0-10° C. The solid was collected by filtration and washed with water (300 mL). The solid was then stirred with MTBE (300 mL) and stirred for 2-4 h at 40-50° C. The temperature was cooled to 10-20° C. over 2-4 h and stirred for 2-4 h. The solid was collected by filtration and washed with MTBE (148 mL). The solid was dried at 45-55° C. for 12 h to obtain title compound (99.36 g, 71%). MS (ES) m/z=276 (M+1).

### Preparation 21 4-Bromo-5-fluoro-6-nitro-1,3-dihydroisobenzofuran-1-ol

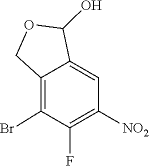 [SMILES: O=[N+]([O-])c1cc2c(c(Br)c1F)COC2O]

4-Bromo-5-fluoro-6-nitroisobenzofuran-1(3H)-one (100 g, 362 mmol) was combined with toluene (865 mL). The reaction was sparged with N₂×3. The reaction temperature was adjusted to −65° C. DIBAL-H (1M in toluene, 510 mL, 510 mol) was added. The reaction was stirred for 1 h at −65° C. EtOAc (495 mL) was added while maintaining the reaction temperature below −60° C. 7% aq. NaHCO₃ solution (250 mL) was added while maintaining the reaction temperature at −60° C. The temperature was adjusted to 10° C. and stirred for 3 hr. The reaction mixture was filtered, and the filter cake was washed with EtOAc (500 mL). To the filtered reaction mixture was added a 25% aq. NaCl solution (450 mL). The mixture was stirred for 0.5 h at 25° C. The layers were allowed to separate, and the upper organic layer was removed and concentrated to 100-200 mL under vacuum below 45° C. EtOAc (500 mL) was added, and the resultant solution concentrated to 100-200 mL under vacuum below 45° C. EtOAc (100 mL) and the resultant solution stirred for 2 h at 20° C. n-Heptane (500 mL) was added and the resultant solution was stirred at 20° C. for 2 h. The mixture was filtered and the isolated solid dried at 45° C. for 20 h to obtain title compound (66.4 g, 66%). MS (ES) m/z=278 (M+1).

### Preparation 22 4-Bromo-5-fluoro-6-nitro-1,3-dihydroisobenzofuran

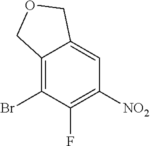 [SMILES: O=[N+]([O-])c1cc2c(c(Br)c1F)COC2]

4-bromo-5-fluoro-6-nitro-1,3-dihydroisobenzofuran-1-ol (100 g, 360 mmol) was combined with DCM (1000 mL) and triethylsilane (60 g, 516 mmol). The reaction mixture was cooled to 5° C. TFA (120 g, 1052 mmol) was added, and the reaction mixture stirred for 30 min at 5° C. The temperature was adjusted to 25° C. and the reaction mixture stirred for 5 h at 25° C. A 10% aq. NaOH solution (400 mL) was added while maintaining a temperature below 30° C. The mixture was stirred for 1 h at 20° C. The mixture was allowed to stand for 1 h and separate. The organic layer was removed concentrated to 100-200 mL below 30° C. EtOAc (400 mL) was added and concentrated to to 100-200 mL below 45° C. EtOAc (800 mL) was added followed by the addition of water (500 mL). The mixture was stirred for 1 h at 20° C. The mixture was allowed to stand for 1 h and allowed to separate. The organic layer was removed concentrated to 100-200 mL below 45° C. n-Heptane (300 mL) was added and concentrated to to 100-200 mL below 45° C.×2. n-Heptane (100 mL) was added followed by EtOAc (50 mL). The temperature was adjusted to 0° C. and stirred for 3 h. The resultant solid was isolated by filtration, the solid washed with n-heptane (100 mL) and dried at 45° C. for 20 h to afford title compound (56.9 g, 60.3%). MS (ES) m/z=(M+1).

### Preparation 23 7-Bromo-6-fluoro-1,3-dihydroisobenzofuran-5-amine

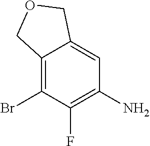 [SMILES: Nc1cc2c(c(Br)c1F)COC2]

MS (ES) m/z=232 (M+1).

### Preparation 24 Ethyl N-[(7-bromo-6-fluoro-1,3-dihydroisobenzofuran-5-yl)carbamothioyl]carbamate

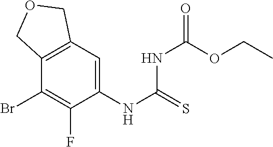 [SMILES: CCOC(=O)NC(=S)Nc1cc2c(c(Br)c1F)COC2]

7-Bromo-6-fluoro-1,3-dihydroisobenzofuran-5-amine (100 g, 431 mmol) was combined with EtOAc (1500 mL) and ethoxycarbonyl isothiocyanate (59.3 g, 452 mmol). The reaction mixture was stirred for 18 h at 10-20° C. The reaction was then concentrated and EtOAc (500 mL) was added followed by heptane (1200 mL). The mixture was stirred for 3 h at 20-25° C. The mixture was filtered. The isolated solid was dried at 40-50° C. for 16 hr to obtain title compound (145.3 kg, 92.8%). MS (ES) m/z=363 (M+1).

### Preparation 25 Ethyl (Z)-(((7-bromo-6-fluoro-1,3-dihydroisobenzofuran-5-yl)amino)(ethylthio)methylene)carbamate

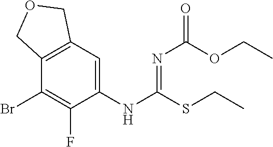 [SMILES: CCOC(=O)/N=C(/Nc1cc2c(c(Br)c1F)COC2)SCC]

Ethyl N-[(7-bromo-6-fluoro-1,3-dihydroisobenzofuran-5-yl)carbamothioyl]carbamate (100 g, 275 mmol) was combined with acetone (761 mL) and K₂CO₃ (114 g, 825 mmol). The mixture was stirred for 30 min at 25° C. Iodoethane (45 g, 288.5 mmol) was added followed by acetone (241 mL). The reaction mixture was stirred for 7 h at 25° C. Water (700 mL) was added and stirred for 30 min at 25° C. The mixture was allowed to stand for 1 h and separate. The layers were separated. To the bottom aqueous layer was added EtOAc (600 mL) and the mixture stirred for 1 h at 25° C. The mixture was allowed to stand for 1 h and separate. The layers were separated. The top organic layers were combined. To the combined organic layers was added 25% NaCl solution (1330 g) and stirred for 30 min at 25° C. The mixture was allowed to stand for 1 h and separate. The layers were separated. The organic layer was concentrated to 100-200 mL below 50° C. under vacuum. EtOAc (600 mL) and water (600 mL) were added and stirred for 30 min at 25° C. The mixture was allowed to stand for 1 h and separate. The layers were separated. The top organic layer was concentrated to 100-300 mL below 50° C. under vacuum. n-Heptane (300 mL) was added and concentrated to 100-300 mL below 50° C. under vacuum× 3. EtOAc (100 mL) was added and stirred at 45° C. under N₂ flow for 10 min. n-Heptane (700 mL) was added dropwise over 3 hr at 45° C. The temperature was adjusted to 25° C. over 3 h. The mixture was stirred for 2 h at 25° C. The mixture was concentrated to 700-800 mL below 30° C. under vacuum. n-Heptane (200 mL) was added and concentrated to 700-800 mL below 30° C. under vacuum. n-Heptane (200 mL) was added and stirred for 3 h at 25° C. The mixture was filtered. The solid was washed with n-Heptane (100 mL) and dried at 50° C. for 12 h to obtain title compound (75 g, 70%). MS (ES) m/z=391 (M+1).

### Preparation 26 6-Bromo-3-(ethylthio)-5-fluoro-7,9-dihydrofuro[3,4-f]quinazolin-1-ol

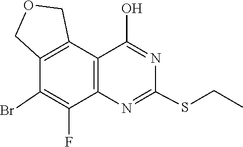 [SMILES: CCSc1nc(O)c2c3c(c(Br)c(F)c2n1)COC3]

Ethyl (Z)-(((7-bromo-6-fluoro-1,3-dihydroisobenzofuran-5-yl)amino)(ethylthio)methylene)carbamate (100 g, 255.6 mmol) was combined with sulfolane (1000 mL), and Eaton's reagent (7.5 wt. % in MSA, 677.47 g, 357.8 mmol). The solution was stirred at 30-40° C. for 30-60 min to get a clear solution. The solution was filtered under vacuum. The solution and NMP (200 mL) were continuously fed through a flow reaction system. The two streams were fed through the first flow reactor heated to 140-170° C. The reaction was fed through the second flow reactor cooled to 40-60° C. The reaction mixture was fed to a holding chamber. The reaction mixture was then fed dropwise to water (2000 mL) at 0-30° C. The mixture was stirred at 0-30° C. for 5-10 h. The mixture was filtered and the solid washed with water (1000 mL). The solid was stirred with water (2000 mL) at 0-30° C. for 5-10 h. The mixture was filtered and the solid washed with water (1000 mL). The solid was stirred with water (1000 mL) at 0-30° C. for 5-10 h. The mixture was filtered and the solid washed with water (1000 mL). The mixture was filtered and the solid dried at 55° C. for 22 h. (64.4 g, 73%). MS (ES) m/z=345 (M+1).

### Preparation 27 6-bromo-1-chloro-3-(ethylthio)-5-fluoro-7,9-dihydrofuro[3,4-f]quinazoline

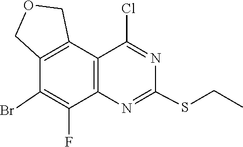 [SMILES: CCSc1nc(Cl)c2c3c(c(Br)c(F)c2n1)COC3]

### Preparation 28 tert-Butyl 8-(6-bromo-3-(ethylthio)-5-fluoro-7,9-dihydrofuro[3,4-f]quinazolin-1-yl)-3,8-diazabicyclo[3.2.1]octane-3-carboxylate

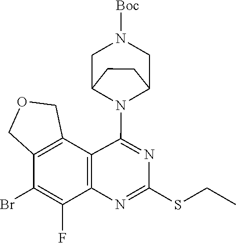 [SMILES: CCSc1nc(N2C3CCC2CN(C(=O)OC(C)(C)C)C3)c2c3c(c(Br)c(F)c2n1)COC3]

### Preparation 29 tert-Butyl 8-(6-(1,3,6,2-dioxazaborocan-2-yl)-3-(ethylthio)-5-fluoro-7,9-dihydrofuro[3,4-f]quinazolin-1-yl)-3,8-diazabicyclo[3.2.1]octane-3-carboxylate

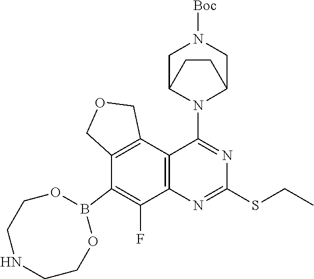 [SMILES: CCSc1nc(N2C3CCC2CN(C(=O)OC(C)(C)C)C3)c2c3c(c(B4OCCNCCO4)c(F)c2n1)COC3]

tert-Butyl 8-(6-bromo-3-(ethylthio)-5-fluoro-7,9-dihydrofuro[3,4-f]quinazolin-1-yl)-3,8-diazabicyclo[3.2.1]octane-3-carboxylate (500 g, 927 mmol) was combined with B₂pin₂ (353 g, 1390 mmol), Pd-117 (79.8 g, 69.5 mmol), Me₄NOAc (247 g, 1.854 mol), and CPME (5 L). The mixture was degassed with nitrogen. The temperature was agitated at 70±5° C. for 15 h. CPME (5 L) was added and the mixture is agitated at 70±5° C. for 10 min. The mixture was filtered through diatomaceous earth. The filtrate was distilled to 5 L and the temperature was adjusted to 50±5° C. Diethanolamine (146 g, 1390 mmol) in IPA (500 mL) was added over 30 min at 50±5° C. tert-butyl 8-(6-(1,3,6,2-dioxazaborocan-2-yl)-3-(ethylthio)-5-fluoro-7,9-dihydrofuro[3,4-f]quinazolin-1-yl)-3,8-diazabicyclo[3.2.1]octane-3-carboxylate seed was added followed by additional diethanolamine Diethanolamine (146 g, 1390 mmol) in IPA (500 mL). The mixture was agitated at 50±5° C. for 1 h. The temperature was adjusted to 5±5° C. and the mixture was agitated at 5±5° C. for 12 h. The product was collected by filtration and dried on the filter for 1 h followed by drying in a tray dryer to obtain title compound (428 g, 80.5%). MS (ES) m/z=505 (M+1, boronic ester degraded to boronic acid in MS).

### Preparation 30 tert-Butyl 8-(6-(2-((tert-butoxycarbonyl)amino)-3-cyano-7-fluorothieno[3,2-c]pyridin-4-yl)-3-(ethylthio)-5-fluoro-7,9-dihydrofuro[3,4-f]quinazolin-1-yl)-3,8-diazabicyclo[3.2.1]octane-3-carboxylate

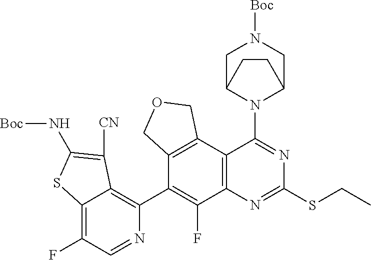 [SMILES: CCSc1nc(N2C3CCC2CN(C(=O)OC(C)(C)C)C3)c2c3c(c(-c4ncc(F)c5sc(NC(=O)OC(C)(C)C)c(C#N)c45)c(F)c2n1)COC3]

tert-Butyl 8-(6-(1,3,6,2-dioxazaborocan-2-yl)-3-(ethylthio)-5-fluoro-7,9-dihydrofuro[3,4-f]quinazolin-1-yl)-3,8-diazabicyclo[3.2.1]octane-3-carboxylate (400 g, 697.54 mmol), was combined with 2-((tert-butoxycarbonyl)amino)-3-cyano-7-fluorothieno[3,2-c]pyridine 5-oxide (228.6 g, 697.5 mmol), Pd-170 (23.44 g, 34.9 mmol), Xphos (16.64 g, 34.9 mmol), NaHCO₃ (1.758 g, 2092.5 mmol) and a solution of CPME/water (6 L/4 L) which was previously sparged with nitrogen. The mixture was agitated at 65±5° C. for 4 h then adjusted to 20±5° C. The phases were allowed to separate, and the aqueous layer was transferred. The organic layer was washed with brine and added to the aqueous layer. The aqueous layer was back extracted with CPME and added to the organic layer. The organic layer was concentrated to 100 mL and MeOH (3.5 L) was added. The organic layer was concentrated to 1.75 L and MeOH (3.15 L) was added. The organic layer was concentrated to 245 L and MeOH (2.25 L) was added. The organic layer was concentrated to 2.45 L and MeOH (1.75 L) was added. The mixture was agitated at 50±5° C. for 1 h. The temperature was adjusted to 20±5° C. and agitated at 20±5° C. for 2 h. The product was collected by filtration and dried on the filter followed by drying in a tray dryer to obtain title compound (462 g, 88.1%). MS (ES) m/z=752 (M+1).

### Preparation 31 tert-Butyl 8-(6-(2-((tert-butoxycarbonyl)amino)-3-cyano-7-fluorothieno[3,2-c]pyridin-4-yl)-3-(ethylsulfonyl)-5-fluoro-7,9-dihydrofuro[3,4-f]quinazolin-1-yl)-3,8-diazabicyclo[3.2.1]octane-3-carboxylate

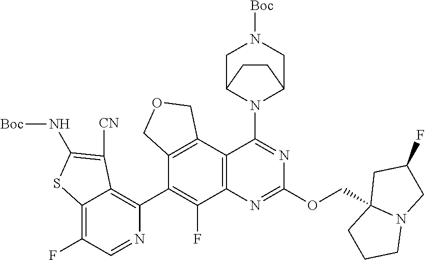 [SMILES: CC(C)(C)OC(=O)Nc1sc2c(F)cnc(-c3c4c(c5c(N6C7CCC6CN(C(=O)OC(C)(C)C)C7)nc(OC[C@@]67CCCN6C[C@H](F)C7)nc5c3F)COC4)c2c1C#N]

tert-Butyl 8-(6-(2-((tert-butoxycarbonyl)amino)-3-cyano-7-fluorothieno[3,2-c]pyridin-4-yl)-3-(ethylthio)-5-fluoro-7,9-dihydrofuro[3,4-f]quinazolin-1-yl)-3,8-diazabicyclo[3.2.1]octane-3-carboxylate (300 g, 1.0 equiv), was combined with DCM:THF (2700:150 mL) under nitrogen and agitated at 20° C. for 15 min. Temperature was adjusted to 0° C. A solution of mCPBA (182.3 g, 1.056 mol) in DCM:THF (1425:75 mL) was added, keeping the temperature below 10° C. The temperature was adjusted to 10° C. and agitated for 4 h at 10° C. A solution of Na₂S₂O₅ (54.1 g, 0.285 mol) in water (600 mL) was slowly added over 15 min, keeping the temperature below 20° C. The temperature adjusted to 10° C. and agitated for 16 h. The mixture was distilled under vacuum at 40° C. to 900 mL. The temperature was adjusted to 20° C. MeOH (1.5 L) was added over 15 min. The temperature was adjusted to 20° C. and agitated for 1 h. A solution of 5 wt % NaHCO₃ (4.5 L) was slowly added over 45 min while keeping the temperature below 30° C. The temperature was adjusted to 20° C. and agitated for 2 h and filtered. The solid was washed with water (1.5 L×2) and dried under nitrogen to obtain title compound (292 g, 98%) as a light yellow solid. MS (ES) m/z=784 (M+1).

### Preparation 32 tert-Butyl 8-(6-(2-((tert-butoxycarbonyl)amino)-3-cyano-7-fluorothieno[3,2-c]pyridin-4-yl)-5-fluoro-3-(((2R,7aS)-2-fluorotetrahydro-1H-pyrrolizin-7a(5H)-yl)methoxy)-7,9-dihydrofuro[3,4-f]quinazolin-1-yl)-3,8-diazabicyclo[3.2.1]octane-3-carboxylate

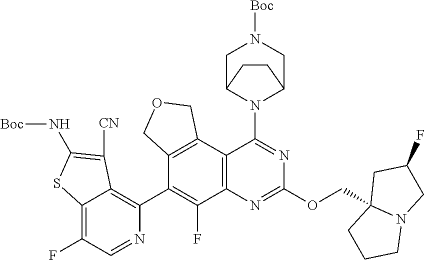 [SMILES: CC(C)(C)OC(=O)Nc1sc2c(F)cnc(-c3c4c(c5c(N6C7CCC6CN(C(=O)OC(C)(C)C)C7)nc(OC[C@@]67CCCN6C[C@H](F)C7)nc5c3F)COC4)c2c1C#N]

tert-Butyl 8-(6-(2-((tert-butoxycarbonyl)amino)-3-cyano-7-fluorothieno[3,2-c]pyridin-4-yl)-3-(ethylsulfonyl)-5-fluoro-7,9-dihydrofuro[3,4-f]quinazolin-1-yl)-3,8-diazabicyclo[3.2.1]octane-3-carboxylate (50 g, 63.8 mmol), was combined with ((2R,7aS)-2 -fluorotetrahydro-1H-pyrrolizin-7a(5H)-yl)methanol (15.23 g, 0.957 mmol), DMAc (250 mL) and agitated at 20±5° C. for 15 min. The temperature was adjusted to 10±5° C. LiOtBu solution (2.2 M in THF, 58 mL, 127.6 mmol) was added while maintaining the temperature ≤20° C. The reaction was stirred for 2 h at 10±5° C. The temperature was adjusted to 0±5° C. A 3% aqueous citric acid solution (500 mL) was slowly added, keeping the temperature ≤10° C. The temperature was adjusted to 20±5° C. EtOAc (500 mL) was added and agitated for 30 min. The agitation was stopped, and the contents allowed to settle for 15 min (layer separation observed in 5 mins). The lower aqueous layer and top organic layer were separated. To the aqueous layer was added EtOAc (250 mL) and agitated at 20±5° C. for 15 min. The agitation was stopped, and the contents allowed to settle for 15 min (layer separation observed in 5 mins). The top organic layer was collected and added to the previously separated organic layer. To the combined organic layers was added a 5% NaHCO₃ solution (500 mL) and agitated at 20±5° C. for 15 min. The agitation was stopped, and the contents allowed to settle for 15 min (layer separation observed in 10 mins). The lower aqueous layer was removed. A solution of 5% LiCl (250 mL) was added and agitated at 20±5° C. for 15 min. The agitation was stopped, and the contents allowed to settle for 15 min (layer separation observed in 5 mins). The lower aqueous layer was removed. A solution of 5% LiCl (250 mL) was added and agitated at 20±5° C. for 15 min. The agitation was stopped, and the contents allowed to settle for 15 min (layer separation observed in 5 mins). The lower aqueous layer was removed. The organic layer was concentrated to 150 mL and swapped with MTBE (250 mL,) and concentrated to an end volume of 150 mL. MTBE (250 mL) was slowly added and warmed to 50° C. n-Heptane (500 mL) was slowly added at 50° C. and a yellow precipitate was observed. The mixture was concentrated to a thick slurry of ~250 mL, swapped with n-heptane (500 mL) to an end volume of 250 mL. n-Heptane (350 mL) was added and the slurry heated to 50° C. The slurry was agitated at 50° C. for 1 h, and cool to ambient temperature and agitated for 3 h. The resultant mixture was filtered, the solid washed with n-heptane (150 mL) and dried in a vacuum oven at 50° C. for 20 h with nitrogen bleed to obtain title compound (50.4 g, 93%). MS (ES) m/z=849 (M+1)

### Preparation 33 4-(1-(3,8-Diazabicyclo[3.2.1]octan-8-yl)-5-fluoro-3-(((2R,7aS)-2-fluorotetrahydro-1H-pyrrolizin-7a(5H)-yl)methoxy)-7,9-dihydrofuro[3,4-f]quinazolin-6-yl)-2-amino-7-fluorothieno[3,2-c]pyridine-3-carbonitrile

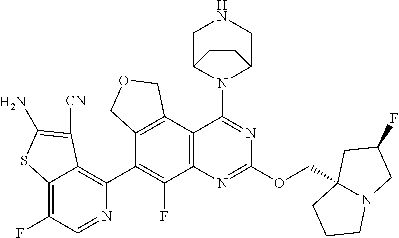 [SMILES: N#Cc1c(N)sc2c(F)cnc(-c3c4c(c5c(N6C7CCC6CNC7)nc(OC[C@@]67CCCN6C[C@H](F)C7)nc5c3F)COC4)c12]

tert-Butyl 8-(6-(2-((tert-butoxycarbonyl)amino)-3-cyano-7-fluorothieno[3,2-c]pyridin-4-yl)-5-fluoro-3-(((2R,7aS)-2-fluorotetrahydro-1H-pyrrolizin-7a(5H)-yl)methoxy)-7,9-dihydrofuro[3,4-f]quinazolin-1-yl)-3,8-diazabicyclo[3.2.1]octane-3-carboxylate (5.0 g, 5.89 mmol) was combined with DCM (15 mL) and cooled to 0-5° C. TFA (13.4 g, 118 mmol) was added over 30 min. The mixture was stirred at 0-5° C. for 4 h. The temperature was adjusted to 20±5° C. The mixture was stirred at 20±5° C. for 12 h. The mixture was concentrated the mixture to ≤15 g. The mixture was cooled to 0-5° C. DMAc (15 mL) was slowly added while maintaining the temperature below 20° C. The mixture was stirred at 20±5° C. for 2 min to get clear solution of title compound TFA salt in DMAc. The solution of crude title compound TFA salt was used directly in the next step. MS (ES) m/z=649 (M+1)

### Preparation 34 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (i.e., Formula II)

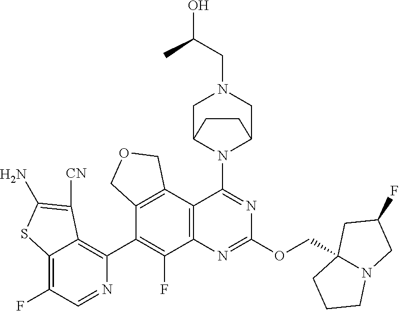 [SMILES: C[C@@H](O)CN1CC2CCC(C1)N2c1nc(OC[C@@]23CCCN2C[C@H](F)C3)nc2c(F)c(-c3ncc(F)c4sc(N)c(C#N)c34)c3c(c12)COC3]

A solution of 4-(1-(3,8-diazabicyclo[3.2.1]octan-8-yl)-5-fluoro-3-(((2R,7aS)-2-fluorotetrahydro-1H-pyrrolizin-7a(5H)-yl)methoxy)-7,9-dihydrofuro[3,4-f]quinazolin-6-yl)-2-amino-7-fluorothieno[3,2-c]pyridine-3-carbonitrile (0.060 g, 0.093 mmol) in DCM (2 mL) and MeOH (2 mL) was treated with (R)-(+)-propylene oxide (0.110 g, 1.850 mmol). The mixture was stirred in a sealed tube at RT for ~60 h, then was concentrated. The residue was purified by preparative HPLC on Luna® C18, 50 mm×250 mm×10 μm, (eluting with 25% to 40% ACN in 10 mM aq. NH₄HCO₃ with 5% MeOH) to obtain the title compound (0.021 g, 24%) as a light-beige solid. MS (ES) m/z=707 (M+1).

### Alternate Synthesis of Formula II 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (i.e., Formula II)

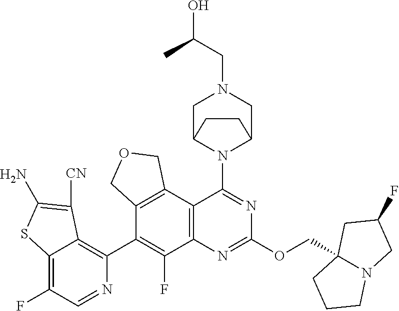 [SMILES: C[C@@H](O)CN1CC2CCC(C1)N2c1nc(OC[C@@]23CCCN2C[C@H](F)C3)nc2c(F)c(-c3ncc(F)c4sc(N)c(C#N)c34)c3c(c12)COC3]

Step 1: Amberchrom™ 50WX8 [H-resin] (4.5 g) was added to a filter and washed with water (2×9 mL) and combined with (R)-1,1-dimethoxypropan-2-ol (3.0 g, 25.0 mmol) and water (4.5 mL). The temperature was adjusted to 50±5° C. and stirred for 2 h. The mixture was cooled to 20±5° C. The resin was filtered off and washed with DMAc (8 mL). The concentration of the (R)-2-hydroxypropanal solution (aldehyde: 1.85 g, 25 mmol) (total weight: 15 g, around 12 wt % aldehyde solution) was calculated.

Step 2: NaOAc (9.66 g, 117.8 mmol) was added to DMAc (10 mL). The mixture was cooled to 0±5° C. A solution of 4-(1-(3,8-Diazabicyclo[3.2.1]octan-8-yl)-5-fluoro-3-(((2R,7aS)-2-fluorotetrahydro-1H-pyrrolizin-7a(5H)-yl)methoxy)-7,9-dihydrofuro[3,4-f]quinazolin-6-yl)-2-amino-7-fluorothieno[3,2-c]pyridine-3-carbonitrile TFA salt in DMAc at 0-5° C. was added while maintaining the internal temperature below 10° C. The temperature was adjusted to 0-5° C. STAB (5 g, 23.6 mmol) was added followed by the addition of the (R)-2-hydroxypropanal solution (aldehyde: 1.85 g, 25 mmol) (total weight: 15 g, around 12 wt % aldehyde solution) over 15 min. The mixture was stirred at 0-5° C. for 30 min. The temperature was adjusted to 20±5° C. and stirred for 2 h. Water (30 mL) was slowly added over 30 min while maintaining the internal temperature below 30° C. Aqueous 10% NH₄OH solution (30 mL) was slowly added over 30 min while maintaining the internal temperature below 20° C. The temperature was to 20±5° C. and stirred for 2 h. The solid was collected by filtration and washed with water (2×20 mL). The solid was dried in a vacuum oven at ≤50° C. overnight to obtain title compound (4.1 g, 97.8%). MS (ES) m/z=707 (M+1).

### Example 1 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate (i.e., Formula I)

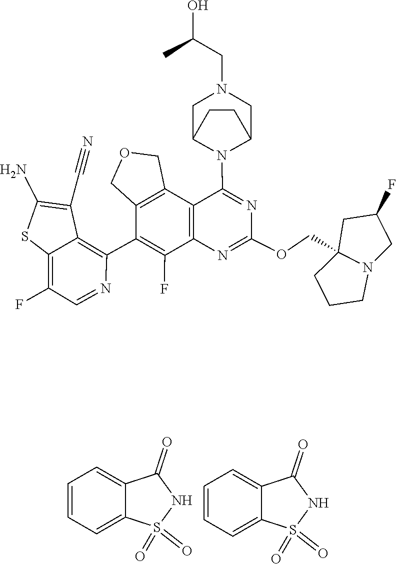

2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (0.177 g, 0.250 mmol) was suspended in EtOH (2 mL). The slurry was heated to 47° C. while stirring at 500 rpm. 2 mL of Saccharin (0.103 g, 0.562 mmol) in EtOH (4 mL) at 47° C. was added to the slurry. All solids dissolved and a clear solution resulted. A white slurry formed after few minutes of stirring. The remaining 2 mL of saccharin solution was added. The solution was stirred for 15 min at 47° C. The solution was cooled to RT. A thick slurry of white solid formed. The solid was isolated by filter paper and dried at 50° C. to obtain the title compound (0.209 g, 78%) as a bright white solid.

### Alternate Synthesis of Formula I 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate (i.e., Formula I)

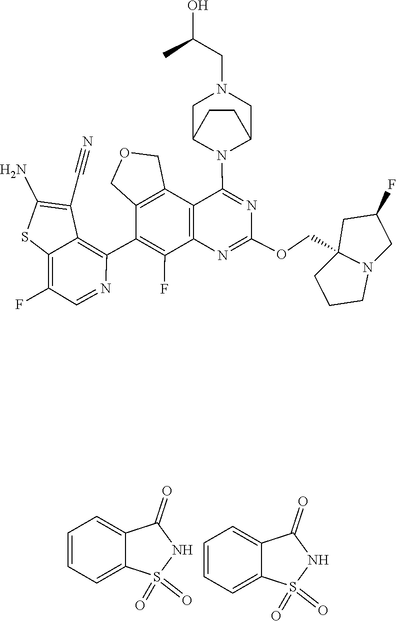

Amberlite IRN78 hydroxide form resin (425 g) was prewashed with THF (2125 mL). 2-amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (425 g, 420.92 mmol) was combined with prewashed Amberlite IRN78 hydroxide form resin (425 g) and THF (4250 mL). The slurry was heated to 50° C. and stirred for 2 h. The slurry was filtered to remove the resin. The filtrate was heated to 50° C. and stirred. To the solution was added Saccharin (220.30 g, 1050 mmol). The reaction was stirred for 10 min. 2-amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate seeds (425 mg, 0.0004 mmol) was added. The slurry was aged at 50° C. for 16 h. The slurry was cooled to 22° C. over 2 h and then aged at 22° C. for 4 h. The slurry was filtered. The solid was washed with 2×THF (850 mL) followed by heptane (850 mL) wash. The solid was dried to a constant weight in a vacuum oven at 22° C. to afford the title compound (384.4 g, 85.1%). ¹H NMR (400 MHz, DMSO-d₆) δ 8.47 (s, 2H), 8.44 (s, 1H), 7.73-7.67 (m, 2H), 7.66-7.57 (m, 6H), 5.66-5.46 (m, 3H), 5.08 (br d, J=12.35 Hz, 1H), 4.74 (br d, J=12.35 Hz, 1H), 4.67-4.53 (m, 3H), 4.44 (br s, 1H), 4.05 (br s, 1H), 4.00-3.70 (m, 4H), 3.55-3.05 (m, 5H), 2.92 (br d, J=19.07 Hz, 1H), 2.70-2.45 (m, 2H), 2.40-2.30 (m, 1H), 2.25-2.00 (m, 6H), 1.98-1.80 (m, 2H), 1.13 (d, J=6.24 Hz, 3H).

### Alternate Synthesis of Formula I 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate (i.e., Formula I)

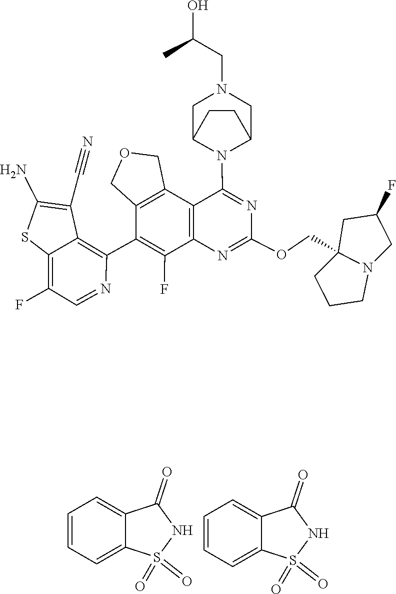

A mixture of saccharin (34.2 kg) and acetone (854.1 kg) were charged to a reactor. The temperature was adjusted to 20° C. and the mixture was agitated until a clear solution formed. The contents were transferred to a second reactor through a 0.45 micron filter. The first reactor was rinsed with acetone (47.5 kg) and the rinse was transferred to the second reactor through a 0.45 micron filter. 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate seed (603.8 g) was added to the second reactor. The temperature of the second reactor was adjusted to 35° C. and the mixture was agitated for 30 min. 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (60.0 kg) and benzyl alcohol (249.6 kg) were added to the first reactor. The temperature of the first reactor was adjusted to 40° C., the mixture was agitated until a clear solution formed, and the temperature was adjusted to 20° C. The contents were transferred to the second reactor through a 0.45 micron filter over at least 4 h while maintaining the temperature of the second reactor at 35° C. The first reactor was rinsed with acetone (47.5 kg) and the rinse was transferred to the second reactor through a 0.45 micron filter while maintaining the temperature of the second reactor at 35° C. The mixture in the second reactor was agitated for at least 2 h, the temperature was adjusted to 5° C. over at least 3 h, and the mixture was agitated for at least 12 h. The solids were collected by filtration, washed with acetone (2×190 kg), and dried for at least 1 h by a stream of nitrogen. The solids were combined with acetone (474 kg, passed through a 0.45 micron filter). The temperature was adjusted to 50° C. The mixture was agitated for at least 5 h, the temperature was adjusted to 20° C. over at least 3 h, and the mixture was agitated for at least 2 h. The solids were collected by filtration, washed with acetone (2×190 kg), dried for at least 1 h by a stream of nitrogen, and dried in a tray dryer for 16 h at a maximum temperature of 65° C. to afford the title compound.

### Alternate Synthesis of Formula I 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate (i.e., Formula I)

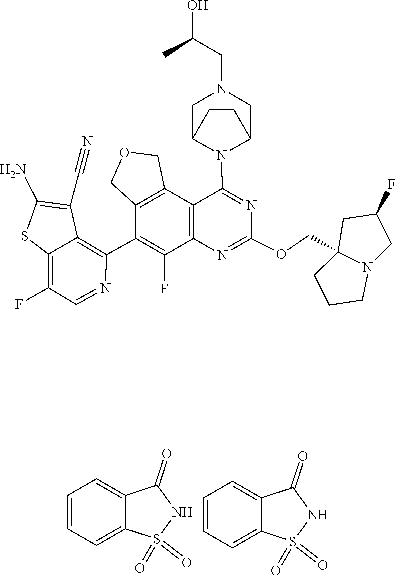

2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (2.7 g, 3.9 mmol) was suspended in tetrahydrofuran (60 mL) in a reactor. The mixture was heated to a jacket temperature of 50° C. and the complete dissolution of solids was observed. Saccharin (1.6 g, 8.7 mmol) was suspended in tetrahydrofuran (30 mL) in a second reactor. The mixture was heated to a jacket temperature of 50° C. and the complete dissolution of solids was observed. A third reactor was heated to a jacket temperature of 50° C. and a portion of the mixture from the first reactor (20 mL) was added over 10 min. Crystalline 2-amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate (seed material; 0.2 g, 1.0 mmol) was added to the third reactor. After 4 h, the remainder of the mixture from the first reactor (40 mL) and the mixture from the second reactor (30 mL) were added to the third reactor simultaneously over 14 h. After 48 h, the solids were collected by filtration, then dried at 60° C. under vacuum to afford the title compound (3.61 g, 87%).

### Alternate Synthesis of Formula I 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate (i.e., Formula I)

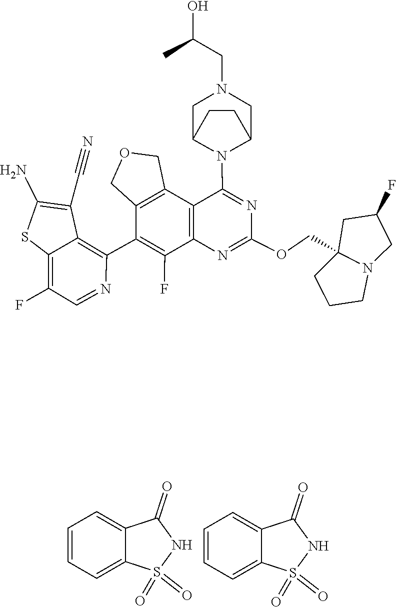

2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (2.7 g, 3.9 mmol) was suspended in tetrahydrofuran (60 mL) in a reactor. The mixture was heated to a jacket temperature of 50° C. and the complete dissolution of solids was observed. Saccharin (3.2 g, 17.6 mmol) was suspended in tetrahydrofuran (30 mL) in a second reactor. The mixture was heated to a jacket temperature of 50° C. and the complete dissolution of solids was observed. A third reactor was heated to a jacket temperature of 50° C. and a portion of the mixture from the first reactor (20 mL) was added over 10 min. Crystalline 2-amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate (seed material; 0.2 g, 1.0 mmol) was added to the third reactor. After 4 h, the remainder of the mixture from the first reactor (40 mL) and the mixture from the second reactor (30 mL) were added to the third reactor simultaneously over 14 h. After 48 h, the solids were collected by filtration, then dried at 60° C. under vacuum to afford the title compound (3.76 g, 91%).

### Example 2 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile 1.25 acetate 0.75 hydrate

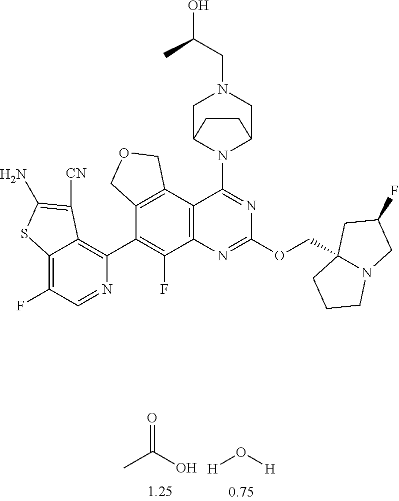

Seed material: A stock solution of 2-amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (151.2 mg) in THF (5 mL) was prepared by stirring the suspension at 50° C. until a clear solution was formed. To 0.50 mL of this solution was added acetic acid (0.020 mL) and n-heptane (0.50 mL). After 48 h, the supernatant solvent was drained from the solids. The solids were washed with n-heptane (1 mL) and dried under vacuum at 40° C. for 24 h to provide seed material.

A mixture of 2-amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (1.01 g) in THF (20 mL) was stirred at 300 rpm at 50° C. until a solution formed. Acetic acid (1.44 mL) was added and the temperature was adjusted to 20° C. over 10 min. Seed material (10.9 mg) was added. n-Heptane (20 mL) was added over 6 h. The temperature was adjusted to 5° C. over 6 h and the mixture was stirred for 4.5 h. The solids were collected by filtration, washed with n-heptane (10 mL), and dried in a vacuum oven at 40° C. for 24 h to afford the title compound as an off-white solid (0.92 g, 81%).

### Example 3 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile monocinnamate

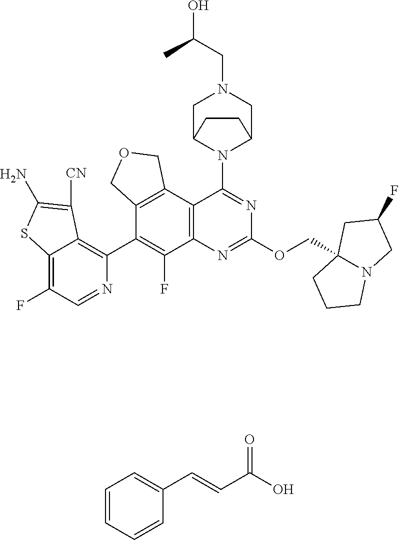 [SMILES: C[C@@H](O)CN1CC2CCC(C1)N2c1nc(OC[C@@]23CCCN2C[C@H](F)C3)nc2c(F)c(-c3ncc(F)c4sc(N)c(C#N)c34)c3c(c12)COC3 ; O=C(O)/C=C/c1ccccc1]

2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (1.07 g, 1.51 mmol) was combined with trans-cinnamic acid (saturated 2-propanol solution; 6 mL) in 2-propanol (4 mL). The suspension was slurried at 55° C. and stirred at 500 rpm overnight. The slurry was filtered. The solids were washed with 2-propanol (2×5 mL). The solids were dried to a constant weight under nitrogen and vacuum to afford the title compound (1.18 g, 91%) as a white solid.

### Example 4 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile 1.5 saccharinate 1.5 THF 0.25 hydrate

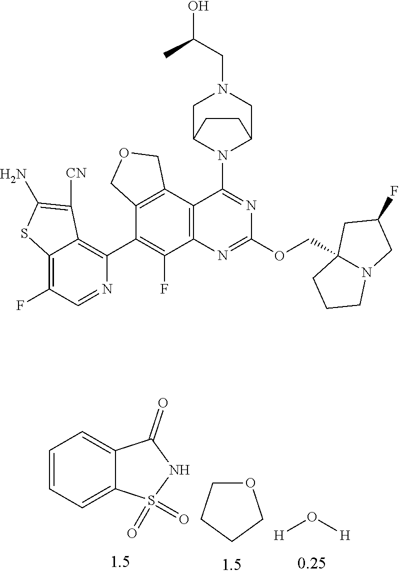

2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile (100 g, 141 mmol) was combined with THF (1750 mL) at 60° C. The material was filtered at this temperature and the solids were rinsed with THF (50 mL). The combined filtrates were adjusted to 50° C. A solution of saccharin (65 g, 355 mmol) in THF (400 mL+50 mL rinse) was added through a 0.45 micron filter, over 10 min. 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate seed (0.100 g) was added. The mixture was stirred at 50° C. for 12 h, then cooled to 20° C. and stirred for 2 h. Solids were collected by filtration and dried in a vacuum oven at 40° C. overnight to give crude product.

Saccharin (0.60 mL, 0.057 mmol; from a stock solution of 87.0 mgs of saccharin in 5 mL of methanol) was added to a portion of the crude product (0.0675 g, 0.069 mmol). Acetone (1 mL) was added and the solution was cooled to 5° C. Mixed crystals of 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile 1.5 saccharinate 1.5 THF 0.25 hydrate and 2-amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile disaccharinate (Formula I) were obtained in approximately two weeks.

### X-Ray Powder Diffraction (XRPD)

The XRPD patterns were obtained on a Bruker D8 Endeavor X-ray powder diffractometer, equipped with a CuKα (1.5418 Å) source and a Linxeye detector, operating at 40 kV and 40 mA. The sample was scanned between 4 and 42 20°, with a step size of 0.009 20° and a scan rate of 0.5 seconds/step, and using 0.3° primary slit opening, and 3.9° PSD opening. The dry powder was packed on a quartz or silicon sample holder and a smooth surface is obtained using a glass slide. The crystal form diffraction patterns were collected at ambient temperature and relative humidity. Crystal peak positions were determined in MDI-Jade after whole pattern shifting based on an internal NIST 675 standard with peaks at 8.853 and 26.774 2θ°.

It is well known in the crystallographic art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

### XRPD of Example 1 (Formula I)

A prepared sample of the crystalline disaccharinate form (i.e., Formula I) is characterized by an XRPD pattern using CuKα radiation as having diffraction peaks (2-theta values) as described in Table 2 below, and in particular having peaks at 4.4±0.2 and 11.3±0.2, in combination with one or more of the peaks selected from the group consisting of 12.9 and 16.2; with a tolerance for the diffraction angles of 0.2 degrees.

In some embodiments, the crystalline form of a compound of Formula I is characterized by having an X-ray powder diffraction (XRPD) pattern comprising peaks at 2θ values of 4.4±0.2 and 11.3±0.2. The crystalline form may be further characterized by comprising peaks at 2θ values of 12.9±0.2 and 16.2±0.2. The crystalline form may be further characterized by comprising peaks at 2θ values of 17.5±0.2, 20.1±0.2, and 20.8±0.2. The crystalline form may still further be characterized by comprising peaks at 2θ values of 4.4±0.2, 11.3±0.2, 17.5±0.2, 20.1±0.2, and 20.8±0.2 or 4.4±0.2, 11.3±0.2, 12.9±0.2, 16.2±0.2, 17.5±0.2, 20.1 0.2, and 20.8±0.2.

TABLE 2
X-ray powder diffraction peaks of Example 1

| | Angle | Relative Intensity |
|---|---|---|
| Peak | (°2-Theta) +/− 0.2° | (% of most intense peak) |
| 1 | 4.4 | 100% |
| 2 | 9.3 | 16.9% |
| 3 | 11.3 | 51.3% |
| 4 | 12.9 | 48.9% |
| 5 | 16.2 | 46.6% |
| 6 | 16.6 | 15.3% |
| 7 | 17.5 | 39.4% |
| 8 | 20.1 | 32.3% |
| 9 | 20.8 | 36.5% |
| 10 | 24.2 | 19.0% |

### XRPD of Example 2 (Crystalline 1.25 Acetate 0.75 Hydrate)

A prepared sample of the crystalline 1.25 acetate 0.75 hydrate form is characterized by an XRPD pattern using CuKα radiation as having diffraction peaks (2-theta values) as described in Table 3 below, and in particular having a peak at 6.0 in combination with one or more of the peaks selected from the group consisting of 14.9, 17.0, and 19.5; with a tolerance for the diffraction angles of 0.2 degrees.

In some embodiments, the crystalline form of the compound of Example 2 is characterized by having an X-ray powder diffraction (XRPD) pattern comprising a peak at 2θ value of 6.0±0.2. The crystalline form may be further characterized by comprising peaks at 2θ values of 14.9±0.2, 17.0±0.2 and 19.5±0.2. The crystalline form may still further be characterized by comprising peaks at 2θ values of 6.0 0.2, 9.2±0.2, 11.8±0.2, and 18.4±0.2 or 6.0±0.2, 14.9±0.2, 17.00.2, 19.5±0.2, 9.2±0.2, 11.8±0.2, and 18.4±0.2.

TABLE 3
X-ray powder diffraction peaks of Example 2

| | Angle | Relative Intensity |
|---|---|---|
| Peak | (°2-Theta) +/− 0.2° | (% of most intense peak) |
| 1 | 6.0 | 89.8% |
| 2 | 9.2 | 64.2% |
| 3 | 11.8 | 53.4% |
| 4 | 13.1 | 41.2% |
| 5 | 13.9 | 23.8% |
| 6 | 14.9 | 76.9% |
| 7 | 16.7 | 29.5% |
| 8 | 17.0 | 84.9% |
| 9 | 18.4 | 47.9% |
| 10 | 19.5 | 100% |
| 11 | 23.1 | 37.8% |

### XRPD of Example 3 (Crystalline Monocinnamate)

A prepared sample of the crystalline monocinnamate form is characterized by an XRPD pattern using CuKα radiation as having diffraction peaks (2-theta values) as described in Table 4 below, and in particular having a peak at 7.4 in combination with one or more of the peaks selected from the group consisting of 4.3, 8.9, and 13.8; with a tolerance for the diffraction angles of 0.2 degrees.

In some embodiments, the crystalline form of the compound of Example 3 is characterized by having an X-ray powder diffraction (XRPD) pattern comprising a peak at 2θ value of 7.4±0.2. The crystalline form may be further characterized by comprising peaks at 2θ values of 4.3±0.2, 8.9±0.2 and 13.8±0.2. The crystalline form may still further be characterized by comprising peaks at 2θ values of 7.4±0.2, 13.0±0.2, 19.5±0.2, and 20.6±0.2 or 7.4±0.2, 4.3±0.2 8.9±0.2, 13.8±0.2 13.0±0.2 19.5±0.2 and 20.6±0.2.

TABLE 4
X-ray powder diffraction peaks of Example 3

| | Angle | Relative Intensity |
|---|---|---|
| Peak | (°2-Theta) +/− 0.2° | (% of most intense peak) |
| 1 | 4.3 | 100% |
| 2 | 7.4 | 12.7% |
| 3 | 8.9 | 13.6% |
| 4 | 10.8 | 2.8% |
| 5 | 13.0 | 7.2% |
| 6 | 13.8 | 3.1% |
| 7 | 14.5 | 2.6% |
| 8 | 17.7 | 4.4% |
| 9 | 18.7 | 2.7% |
| 10 | 19.5 | 7.5% |
| 11 | 20.6 | 8.3% |

### Example 5

### In-Vitro Dissolution Test of API/Polymer Film Cast(s)

1. Set centrifuge at 37° C.
2. Place samples in Vortemp vortex mixer at 37° C. approximately 10 minutes prior to beginning dissolution experiment.
3. Pipette 1.5 mL of ~37° C. SGF to each vial. Theoretical concentration of API is 1.5 mg/mL
4. Mix samples for 15 minutes.
5. Filter 0.170 mL of each sample using 0.45 um centrifuge filter.
6. Dilute 0.1 mL of filtrate of each sample with 0.4 mL of 10% water/90% MeOH directly in an HPLC vial. This is a dilution factor of 5.
7. At 20 min, dilute the samples in the Vortemp with 3 mL ~37° C. FaSIE This is time zero. Theoretical API concentration is 0.5 mg/mL.
8. At 10, 30, 60, 120, and 240 minutes, take 0.3 mL sample and filter using 0.45 centrifuge filter. Dilute 0.1 mL filtrate with 0.4 mL of 10% water/90% MeOH directly in an HPLC vial. This is a dilution factor of 5.
9. Submit all samples for HPLC analysis.
10. Plot Concentration vs. Time for each sample evaluated. Report as supersaturation results.

In this example, it was used 0.01N HCl or water as the stomach. 4 mg/mL active, that is Formula II or Formula I, was the target concentration in 2.25 mL 0.01N HCl or DIW. 0.01N HCl=pH2 which is approx. the pH of a normal healthy human stomach. DIW=pH 5.5, approximately. This represents the pH of a patient on an acid reducing agent, is ill, old, or has achlorhydria.

TABLE 5

| | Formula II | Formula I |
|---|---|---|
| | Concentration | Concentration |
| Aqueous Media | (mg/mL) | (mg/mL) |
| 0.01N HCl | 1.792 | 2.427 |
| Simulated Intestinal Fluid | 0.611 | 1.123 |
| (Fasted) 2 hr | | |
| Deionized Water (DIW) | 0.004 | 2.429 |
| Simulated Intestinal Fluid | 0.269 | 0.87 |
| (Fasted) 2 hr | | |

The above Table 5 contains comparative solubility data for the Formula II and the Formula I. It is expected that the improved solubility of Formula I, when compared to Formula II, will facilitate the administration of a higher dose of the compound of Formula I, if necessary. The compound of Formula I immediately and completely dissolves in the stomach.

### Example 6

Beagle Dogs and naïve Cynomolgus Monkeys were orally dosed in a cross-over animal pharmacokinetic study with Formula I at 30 mg/kg or 60 mg/kg filled in capsules (enteric and non-enteric). Multiple capsules were administered (when necessary) to achieve the target dose based on each animal's weight. For fasted studies, animals were fasted overnight and offered food 2 hours post-dosing. For non-fasted studies, animals were dosed with water for injection (WFI) immediately after capsule dosing and offered food.

Blood samples were collected at 0.25, 0.5, 1, 2, 4, 8, 24, 48, and 72 hours for oral dosing. Cage side observations were performed twice daily and on an unscheduled basis as needed. Cage side observations included mortality, moribundity, general health, emesis, and signs of toxicity. Clinical observations and body weight were taken prior to dosing.

Free plasma concentrations of Formula I were analyzed using a LC-MS/MS method. WinNonlin (Phoenix™, version 8.3) was used for pharmacokinetic calculations. The following pharmacokinetic parameters were calculated from the plasma concentration versus time data: AUCₗₐₛₜ and AUC_{inf}.

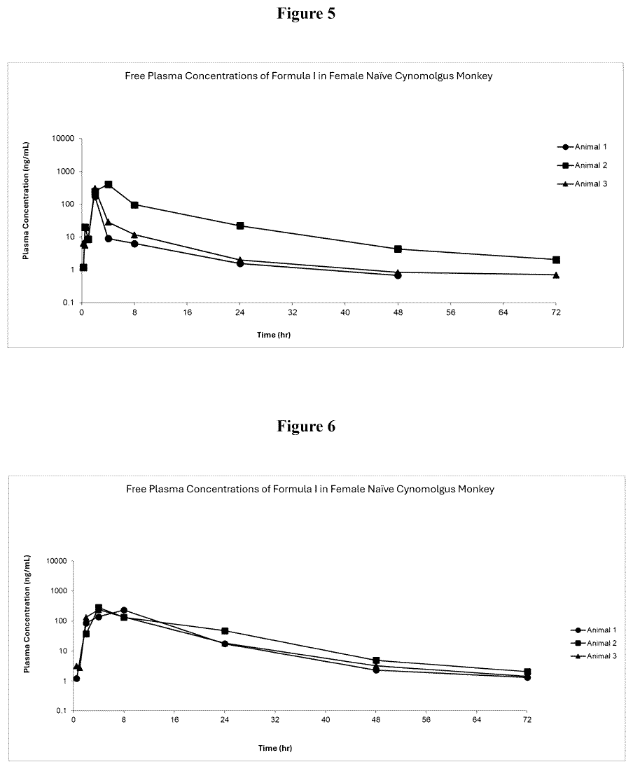

The results are summarized in Tables 6-8 and FIGS. 4-6.

TABLE 6
Summary of Emesis Events (Time after
dosing and Amount) in Dogs

| | Non-enteric Capsules | | Enteric Capsules |
|---|---|---|---|
| Animal | (Fasted) | | (Fasted) |
| 1 | 53 min | (5 mL) | None |
| 2 | 39 min | (10 mL) | None |
| 3 | 29 min | (10 mL) | 73 min (15-20 mL + bits of capsule) |
| 4 | 23 min | (10 mL) | 32 min (15-20 mL + bits of capsule) |
| 5 | 27 min | (10 mL) | None |
| 6 | 28 min | (10 mL) | 53 min (10 mL) |
| 7 | 28 min | (10 mL) | None |
| | 31 min | (10 mL) | |
| | 32 min | (5 mL) | |
| 8 | None | | None |
| 9 | 19 min | (10 mL) | 24 min (10 mL + 1.5 capsules) |
| | 25 min | (10 mL) | 29 min (10 mL + 1 capsule) |
| 10 | 28 min | (5 mL) | None |
| | 33 min | (10 mL) | |
| 11 | None | | None |
| 12 | 38 min | (5 mL) | None |

Male Beagle Dogs were dosed with Formula I (30 mg/kg, Oral) in Capsules
Non-enteric Capsules = HPMC Capsules
Enteric Capsules = Eudracaps ® Capsules
Amounts of emesis are approximate

The above Table 6 shows that emesis was significantly reduced in fasted dogs dosed with enteric capsules comprising Formula I compared to non-enteric capsules comprising Formula I. Specifically, eight animals that received enteric capsules comprising Formula I showed no emesis events.

TABLE 7
Summary of Emesis Events (Time after dosing) in Monkeys

| | Non-enteric Capsules | Enteric Capsules |
|---|---|---|
| Animal | (Fasted) | (Fasted) |
| 1 | 2 hrs | None |
| 2 | None | None |
| 3 | 4 hrs | None |

Female Naïve Cynomolgus Monkeys were dosed with Formula I (60 mg/kg, Oral) in Capsules
Non-enteric Capsules = HPMC Capsules
Enteric Capsules = Eudracaps ® Capsules

The above Table 7 shows that no emesis events were observed in fasted monkeys dosed with enteric capsules comprising Formula I. In contrast, emesis was observed in two out of the three fasted monkeys that were dosed with non-enteric capsules comprising Formula I.

TABLE 8
Exposure Data from Free Plasma Concentration Curves in FIG. 4

| | AUCₗₐₛₜ | AUC_{inf} |
|---|---|---|
| Dose Form | (nM * hr) | (nM * hr) |
| Non-enteric Capsules | 1245 +/− 1356 | 1273 +/− 1368 |
| Enteric Capsules | 2645 +/− 324 | 2673 +/− 327 |

Fasted Female Naïve Cynomolgus Monkeys were dosed with Formula I (60 mg/kg, Oral) in Capsules
Non-enteric Capsules = HPMC Capsules
Enteric Capsules = Eudracaps ® Capsules

The above Table 8 shows a higher AUC exposure and lower variability in fasted monkeys dosed with enteric capsules comprising Formula I compared to non-enteric capsules comprising Formula I.

FIGS. 4, 5, and 6 show a higher AUC exposure and lower variability in fasted monkeys dosed with enteric capsules comprising Formula I compared to non-enteric capsules comprising Formula I.

1-42. (canceled)

43. A compound of the formula: 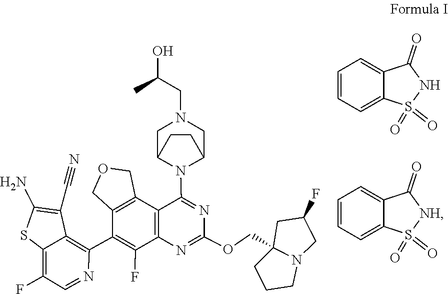
wherein solvent may be optionally present.

44. A crystalline form of a compound of claim 43, wherein the crystalline form is characterized by having an X-ray powder diffraction (XRPD) pattern comprising peaks at 2θ values of 4.4±0.2 and 11.3±0.2.

45. The crystalline form according to claim 43, wherein the crystalline form is characterized by having an X-ray powder diffraction (XRPD) pattern further comprising peaks at 2θ values of 12.9±0.2 and 16.2±0.2.

46. The crystalline form according to claim 43, wherein the crystalline form is characterized by having an X-ray powder diffraction (XRPD) pattern further comprising peaks at 2θ values of 17.5±0.2, 20.1±0.2, and 20.8±0.2.

47. A pharmaceutical composition comprising a compound of claim 43, and at least one excipient.

48. A method of treating a patient for cancer, comprising administering to a patient in need thereof, an effective amount of a pharmaceutical composition according to claim 47.

49. A method of treating a patient for cancer, comprising administering to a patient in need thereof, an effective amount of a compound according to claim 43.

50. The method according to claim 48, wherein the cancer is selected from lung cancer, pancreatic cancer, cervical cancer, esophageal cancer, endometrial cancer, ovarian cancer, cholangiocarcinoma, or colorectal cancer.

51. The method according to claim 49, wherein the cancer is selected from lung cancer, pancreatic cancer, cervical cancer, esophageal cancer, endometrial cancer, ovarian cancer, cholangiocarcinoma, or colorectal cancer.

52. The method according to claim 49, wherein the patient is also administered an effective amount of one or more of a PD-1 inhibitor, a PD-L1 inhibitor, a CDK4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof.

53. A method of preparing a compound of claim 43, the method comprising suspending 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile in a solvent, adding saccharin and isolating the disaccharinate salt.

54. The method of claim 53, wherein the saccharin is dissolved in the solvent, before it is added to the 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile in the solvent.

55. The method according to claim 54, wherein the solvent is an alcohol.

56. The method according to claim 55, wherein the alcohol is a C₁-C₄ alcohol.

57. The method according to claim 55, wherein the solvent comprises at least one of ethanol or methanol.

58. The method according to claim 53, wherein the saccharin is dissolved in the solvent, and the resulting solution is added portionwise to the 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile in the solvent.

59. The method according to claim 58, wherein the solvent is ethanol and 2-Amino-7-fluoro-4-(5-fluoro-3-(((2R,7aS)-2-fluorotetrahydro-1H-pyrrolizin-7a(5H)-yl)methoxy)-1-((1R,5S)-3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile in ethanol is heated to about 47±5° C., before the saccharin in ethanol is added.

60. A solid oral pharmaceutical dosage form comprising:
a) a core composition comprising a compound of the formula: 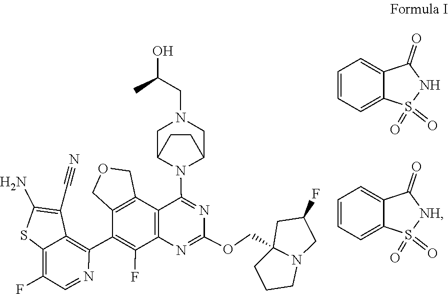
b) a capsule that contains the core composition and wherein the capsule has a body and a cap;
c) a polymeric seal covering the transition between the capsule cap and body; and
d) an enteric coating that coats the polymeric seal and capsule.

61. A solid pharmaceutical composition, comprising: (1) a compound of claim 43 constituting about 30-67% percent of the composition; (2) microcrystalline cellulose constituting about 20-55% of the composition; (3) mannitol constituting about 0-10% of the composition; (4) crospovidone constituting about 2% to about 5% of the composition; (5) colloidal silicon dioxide constituting up to about 0.5-2% of the composition; (6) magnesium stearate constituting about 1-3% of the composition, and (7) an enteric coating, wherein all percentages are percentages by weight and wherein the total weight is 100%.
